(19) United States
(12) Patent Application Publication
(10) Pub. No.: US 2019/0106722 A1
(43) Pub. Date: Apr. 11, 2019

(54) Production of Glycosylated Melanin Precursors in Recombinant Hosts

(71) Applicant: EVOLVA SA, REINACH (CH)

(72) Inventors: Laura Occhipinti, Reinach (CH); Yiming Chang, Reinach (CH); Jorgen Hansen, Reinach (CH)

(21) Appl. No.: 16/095,564

(22) PCT Filed: Apr. 12, 2017
(86) PCT No.: PCT/EP2017/058852

§ 371 Date: Oct. 22, 2018

Related U.S. Application Data
(60) Provisional application No. 62/326,461, filed on Apr. 22, 2016.

(51) Int. Cl. C12P 19/60; C12N 9/10; C12N 9/02

(52) U.S. Cl. CPC C12P 19/60; C12N 9/0071; C12N 9/1048

(57) ABSTRACT

The invention relates to methods for producing melanin and melanin precursors, derivatives, and intermediates. In particular, recombinant microorganisms are disclosed that express tyrosinases to produce 5,6-DHI and express UGT polypeptides capable of either in vivo or in vitro glycosylation of melanin precursors, derivatives, and intermediates. Glycosylated 5,6-DHI is produced both in vivo and in vitro.

### CROSS-REFERENCE TO RELATED APPLICATIONS

This application claims the benefit of U.S. Provisional Patent Application No. 62/326,461, filed Apr. 22, 2016, which is incorporated by reference herein in its entirety.

### BACKGROUND OF THE INVENTION

### Field of the Invention

This disclosure relates to recombinant production of melanin precursors and glycosylated melanin precursors, such as glycosylated 5,6-dihydroxyindole (DHI), and derivatives thereof, in recombinant hosts, particularly yeast.

### Description of Related Art

Melanin represents the principal molecule that gives black hair its color. For the purpose of gentle, elegant, and natural hair dying, it would be desirable to produce a soluble melanin or a melanin precursor that could be applied to hair and converted in situ to black colored aggregates. However, the production of useful melanin is not without its difficulties.

Chemically synthesized melanin, while easily produced, immediately forms aggregates/precipitates that can only be re-solubilized under very high pH conditions leading to significant application challenges. Other sources of melanin include extraction from fermentation leachates by repetitive trophic cycling in the controlled conditions of primary and secondary bioreactors where nutrients are cycled between microorganisms such as bacteria, yeast and fungi and black soldier fly larvae to isolate the melanins. Melanin has also been produced using the bacterium, Escherichia coll. However, such processes are expensive, complex, and require additional purification steps to isolate useful melanin.

Melanin is a polymerization product of 5,6-dihydroxyindole (5,6-DHI) and its 2-carboxylic acid (5,6-DHICA) which spontaneously forms over several steps upon oxidation of L-3,4-dihydroxyphenylalanine (L-DOPA) (see FIG. 1). L-DOPA is a derivative of tyrosine produced by the action of tyrosinases, which catalyze both the meta-hydroxylation of L-tyrosine to L-DOPA as well as its subsequent oxidation to DOPAquinone. The reactive DOPAquinone generated spontaneously transforms into leucoDOPAchrome (cycloDOPA), which subsequently oxidizes to DOPAchrome. The main precursors of melanin, 5,6-DHI and 5,6-DHICA, each originate from DOPAchrome.

Kinetic analyses of the melanin biosynthetic pathway suggest that the formation of L-DOPA from L-tyrosine is slow compared to the formation of DOPAquinone and DOPAchrome. Furthermore, the formation of 5,6-DHI and 5,6-DHICA from DOPAchrome also occurs slowly leading to a product ratio favorably shifted toward 5,6-DHI. The final step of 5,6-DHI polymerization to eumelanin is spontaneous. Therefore, a mechanism to govern this step may be useful for producing desired soluble melanin or melanin precursors in a controlled way.

Glycosylation of 5,6-DHI monomers may be a useful mechanism to prevent this spontaneous polymerization. Either or both of the hydroxyl residues in position 5 and 6 of 5,6-DHI may be glycosylated to form mono- or di-O-glycosylated 5,6-DHI (see FIGS. 2 and 3). While Saccharomyces cerevisiae yeast (budding yeast) is capable of small molecule glycosylation, it lacks the melanin biosynthetic pathway. Thus, a yeast-based system for production of useful melanin precursors can satisfy the need in the art of a new way of producing useful melanin and/or melanin precursors that can be used for in situ generation of black hair color and related applications.

### SUMMARY OF THE INVENTION

It is against the above background that the present invention provides certain advantages and advancements over the prior art. In particular, as set forth herein, the use of recombinant microorganisms to make melanin precursors and glycosylated melanin precursors is disclosed.

Although this invention disclosed herein is not limited to specific advantages or functionalities, in a first aspect, the invention provides a recombinant host including an operative engineered biosynthetic pathway including a heterologous gene encoding a tyrosinase polypeptide, wherein the tyrosinase polypeptide is capable of catalyzing formation of a melanin precursor from tyrosine. In one embodiment, the melanin precursor is a hydroxyindole.

In a second aspect, a recombinant host includes an operative engineered biosynthetic pathway including a heterologous gene encoding a tyrosinase polypeptide, wherein the tyrosinase polypeptide is capable of catalyzing formation of a dihydroxyindole.

In a third aspect, a recombinant host includes an operative engineered biosynthetic pathway including a first heterologous gene encoding a tyrosinase polypeptide and a second heterologous gene encoding a glycosyltransferase (UGT) polypeptide, wherein the tyrosinase polypeptide is capable of catalyzing formation of a dihydroxyindole and the UGT polypeptide is capable of glycosylating the dihydroxyindole.

In a fourth aspect, a recombinant host includes (a) a gene encoding a first polypeptide capable of catalyzing the formation of 5,6-dihydroxyindole (DHI), and (b) a gene encoding a glycosyltransferase (UGT) polypeptide. The UGT polypeptide is capable of glycosylation of 5,6-DHI, at least one of the genes is a recombinant gene, and the recombinant host produces a glycosylated 5,6-DHI. In one embodiment of the fourth aspect, the first polypeptide comprises a tyrosinase polypeptide having at least 50% identity to an amino acid sequence set forth in SEQ ID NO: 2, 4, 6, 8 or 10, and the UGT polypeptide comprises a UGT polypeptide having at least 50% identity to an amino acid sequence set forth in SEQ ID NO: 12, 14, 16, 18, 20, 22, 24, 26, 28, 30, 32, 34, 36, 38, 40, 42, 44, 46, 48, 50, or 52.

In a fifth aspect, the invention provides a method for producing glycosylated 5,6-DHI including (a) growing the recombinant host according to any one of the first, second, third, fourth, eighth, ninth, or tenth aspects in a culture medium, wherein a glycosylated DHI is synthesized by the recombinant host; and (b) optionally isolating the glycosylated DHI.

In one embodiment of the fifth aspect, the recombinant host comprises a yeast cell, a plant cell, a mammalian cell, an insect cell, a fungal cell, or a bacterial cell. In another embodiment of the fifth aspect, the recombinant host is a bacterial cell that is an Escherichia cell, a Lactobacillus cell, a Lactococcus cell, a Cornebacterium cell, an Acetobacter cell, an Acinetobacter cell, or a Pseudomonas cell. In a further embodiment of the fifth aspect, the recombinant host is a yeast cell that is from a Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species. In a particular embodiment of the fifth aspect, the recombinant host is a yeast cell that is a cell from the Saccharomyces cerevisiae species.

In a sixth aspect, the invention provides a method for producing glycosylated 5,6-DHI from a bioconversion reaction including (a) growing a recombinant host in a culture medium, wherein the host expresses a gene encoding a UGT polypeptide capable of glycosylation of a melanin precursor; (b) adding a melanin precursor comprising 5,6-DHI to the culture medium to induce glycosylation of the melanin precursor; and (c) optionally isolating the glycosylated 5,6-DHI. In one embodiment, the method according to the sixth aspect further includes isolating the UGT polypeptide from the recombinant host prior to addition of the melanin precursor. In another embodiment of the sixth aspect, the melanin precursor is glycosylated in an in vitro reaction. In one embodiment of the sixth aspect, the UGT polypeptide comprises a UGT polypeptide having at least 50% identity to an amino acid sequence set forth in SEQ ID NO: 12, 14, 16, 18, 20, 22, 24, 26, 28, 30, 32, 34, 36, 38, 40, 42, 44, 46, 48, 50, or 52.

In a seventh aspect, a method for producing glycosylated 5,6-DHI from an in vitro reaction includes contacting 5,6-DHI with one or more UGT polypeptides in the presence of one or more UDP-sugars. In one embodiment of the seventh aspect, the UGT polypeptide comprises a UGT polypeptide having at least 50% identity to an amino acid sequence set forth in SEQ ID NO: 12, 14, 16, 18, 20, 22, 24, 26, 28, 30, 32, 34, 36, 38, 40, 42, 44, 46, 48, 50, or 52. In another embodiment of the seventh aspect, the one or more UDP-sugars comprise plant-derived or synthetic glucose.

In an eighth aspect, a recombinant host includes an operative engineered biosynthetic pathway having one or more heterologous genes, wherein each of the one or more heterologous genes encodes a polypeptide capable of catalyzing formation of a melanin precursor from tyrosine. In one embodiment of the eighth aspect, the melanin precursor is a hydroxyindole.

In a ninth aspect, a recombinant host includes an operative engineered biosynthetic pathway having one or more heterologous genes, wherein each of the one or more heterologous genes encodes a polypeptide capable of catalyzing formation of a dihydroxyindole.

In a tenth aspect, a recombinant host includes an operative engineered biosynthetic pathway including one or more heterologous genes wherein each of the one or more heterologous genes encodes a polypeptide capable of catalyzing the formation of a melanin precursor from tyrosine and one or more heterologous genes each encoding a glycosyltransferase (UGT) polypeptide. The melanin precursor is a dihydroxyindole, and each of the UGT polypeptides is capable of glycosylating the dihydroxyindole. In one embodiment of the tenth aspect, the host is capable of producing a glycosylated dihydroxyindole. In another embodiment of the tenth aspect, the glycosylated dihydroxyindole is mono-glucosylated 5,6-DHI in position 5 (β-D-5Glc-6OH-indole; C1), mono-glucosylated 5,6-DHI in position 6 (C2), or di-glucosylated 5,6-DHI. In one embodiment of the tenth aspect, the host is capable of producing a plurality of glycosylated dihydroxyindoles.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 represents a schematic of the eumelanin biosynthetic pathway. Chemical reactions are numbered 1-8. Enzymes are indicated where applicable at each reaction. Tyrp2: tyrosinase-related protein 2 shifts the equilibrium in favor of 5,6-DHICA and contains zinc ions. Tyrp1: tyrosinase-related protein 1,5,6-DHICA oxidase promotes melanin formation from 5,6-DHICA and contains iron ions;

FIG. 2 shows the chemical structure of 5,6-dihydroxyindole (DHI). The active hydroxyl groups are circled;

FIG. 3 shows the chemical structures of glucosides derived from 5,6-DHI. From left to right: mono-glucosylated 5,6-DHI in position 5 (β-D-5Glc-6OH-indole; C1); mono-glucosylated 5,6-DHI in position 6 (β-D-5OH-6Glc-indole, C2); (β-D-5Glc-6Glc-indole, double Glc).

FIG. 4 illustrates results of a drop test of yeast strains transformed with tyrosinase genes. Strain IDs and organisms are shown. Strain YN077 carrying an empty vector is shown as negative control. Strains YN013, YN014, YN075 and YN076 (containing respectively Pholiota nameko TYR-2, Pycnoporus sanguineus TYR, L. edodes TYR and P. nameko TYR-1 tyrosinases), are positive for pigment formation;

FIG. 5 shows enrichment of tyrosine increased browning of yeast cells. FIG. 5A: Drop test of yeast strains containing tyrosinase genes. Cells were dropped on plates containing 1.42 mM tyrosine. Strain IDs are reported on the left. FIG. 5B: Liquid medium cultures containing 1.42 mM tyrosine of strains YN013 and YN014 after 1, 2 and 3 days of incubation at 30° C. under shaking. Right column: control culture in standard medium (0.42 mM tyrosine); Left column: medium with 1.42 mM tyrosine;

FIG. 6 shows precursor feeding (5,6-DHI) of cells containing UGTs. FIG. 6A shows a pictorial representation of the precursor feeding experiment. Wild type cells carrying plasmids containing UGTs were fed with the precursor 5,6-DHI, obtaining as a final product, glycosylated melanin precursors (GLYMPs). FIG. 6B. Left: control medium supplemented with 5,6-DHI (210 μg/ml) and C1 at 2 different concentrations (100 and 200 μg/ml). Images of cultures, supernatants and pellets of fed strains. Plasmid IDs (Pl. ID), UGT genes and strains IDs are listed;

FIG. 7 shows precursor feeding on strains containing UGTs leads to GLYMPs formation. Strain numbers and correspondent UGTs are shown. FIG. 7A: GLYMPs in the medium (supernatant). FIG. 7B: GLYMPs in the pellet-soluble fraction of extracted yeast cells;

FIG. 8 shows a LC_MS chromatogram of YN101 with the Y-axis representing signal intensity and the X-axis representing time. Mass Spectrometry detector was a Single Quadrupole. Top: chromatogram=C1 standard at 500 ng/mL, bottom: chromatogram=YN101 sample;

FIG. 9 shows a LC_MS chromatogram of YN108 with the Y-axis representing signal intensity and the X-axis representing time. Mass Spectrometry detector was a Single Quadrupole. The three chromatograms on top show the three standards injected individually (Di-Glc, C1, C2, being the double glycosylated and the two mono-glycosylated compounds) followed by the co-injection of the three standards all together, in the concentration of 500 ng/ml each. Injection volume was 5 microliters for all samples. YN108-SIR-310 shows the peaks obtained from the cell extract of YN108. All the three peaks are detectable at the expected retention times and predicted masses for the YN108 sample (bottom) indicating production of all three GLYMPs: Di-Glc, C1, and C2 by YN108;

FIG. 10A shows a LC-MS chromatogram for YN108 with the Y-axis representing signal intensity and the X-axis representing time. Mass spectrometry detector was a Time-Of-Flight (TOF). The three chromatograms on top show the three standards injected individually (Di-Glc, C1, C2, being the double glycosylated and the two mono-glycosylated compounds) followed by the co-injection of the three standards all together, in the concentration of 500 ng/ml. Injection volume was 5 microliters for all samples. YN108-EIC 310.09 shows the peaks obtained from the cell extract of YN108. All the three peaks are detectable at the expected retention times and predicted masses for the YN108 sample (bottom) indicating production of all three GLYMPs: Di-Glc, C1, and C2 by YN108;

FIG. 10B shows high-resolution mass spectra of the peaks at the indicated Retention Times. The order of the spectra is the same as FIG. 10A (top three spectra are the standards and bottom three are the samples). The observed signals are in agreement with the expected m/z (mass/charge) values, and there is perfect correlation between the spectra of the standards (for Di-Glc, the m/z of the [M−H]⁻ ion is 472 and the m/z of the [M+HCOOH—H]⁻ ion is 518; for C1 and C2, the m/z of the [M−H]⁻ ion is 310) and the spectra of the YN108 sample confirming the production of all three GLYMPs (the m/z of the [M−H]⁻ ion in the Di-Glc spectrum of the sample is not observed due to sample matrix effect);

FIG. 11 illustrates a yeast expression plasmid utilized for tyrosinase in vivo expression (see Mumberg et al., Yeast vectors for the controlled expression of heterologous proteins in different genetic backgrounds, Gene 156(1):119-22, 1995) based on pRS316 and modified with the insertion of a PGK1 and ADH2 yeast promoter and terminator, respectively. This plasmid carries the URA3 auxotrophic marker;

FIG. 12 illustrates an E. coli expression vector used for UGT gene expression in an in vitro system. The plasmid was synthesized by GeneArt™ gene synthesis. It carries a T7 promoter and a T7 terminator; and

FIG. 13 illustrates a yeast expression plasmid (see Mumberg et al., Yeast vectors for the controlled expression of heterologous proteins in different genetic backgrounds, Gene 156(1):119-22, 1995) based on pRS315 and modified with the insertion of a yeast TEF1 promoter, a yeast ENO2 terminator, and a LEU2 auxotrophic marker. This plasmid was utilized for UGT in vivo expression in yeast.

### DETAILED DESCRIPTION OF THE INVENTION

All publications, patents and patent applications cited herein are hereby expressly incorporated by reference in their entirety for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that the genome of a recombinant host described herein can be augmented through stable introduction of one or more recombinant genes or through the introduction of recombinant genes via plasmidic DNA. Generally, introduced DNA is not originally resident in the host that is the recipient of the DNA. However, it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species, or can be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. Said recombinant genes are particularly encoded by cDNA.

As used herein, the terms “codon optimization” and “codon optimized” refer to a technique to maximize protein expression in fast-growing microorganisms such as E. coli or S. cerevisiae by increasing the translation efficiency of a particular gene. Codon optimization can be accomplished, for example, by transforming nucleotide sequences of one species (a gene donor species) into the genetic sequence of a different species (a recombinant host or gene acceptor species). For example, a recombinant gene from a first species may be codon optimized for a recombinant host that is a different species for optimal gene expression. Optimal codons help to achieve faster translation rates and high accuracy. Because of these factors, translational selection is expected to be stronger in highly expressed genes.

As used herein, the term “engineered biosynthetic pathway” refers to a biosynthetic pathway that occurs in a recombinant host, as described herein, and does not naturally occur in the host.

As used herein, the term “endogenous” gene refers to a gene that originates from and is produced or synthesized within a particular organism, tissue, or cell.

As used herein, the terms “heterologous sequence,” “heterologous coding sequence,” and “heterologous gene” are used to describe a sequence derived from a species other than the recombinant host that encodes a polypeptide. In some embodiments, the recombinant host is a S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different from the recombinant host expressing the heterologous sequence. In some embodiments, a coding sequence is a sequence that is native to the host.

As used herein, the terms “variant” and “mutant” are used to describe a protein sequence that has been modified at one or more amino acids, compared to the wild-type sequence of a particular protein.

As used herein, the terms “glycosylation,” “glycosylate,” “glycosylated,” and “protection group(s)” can be used to refer to aspects of the chemical reaction in which a carbohydrate molecule is covalently attached to a hydroxyl group or attached to another functional group in a molecule capable of being covalently attached to a carbohydrate molecule. The term “mono” used in reference to glycosylation refers to the attachment of one carbohydrate molecule. The term “di” used in reference to glycosylation refers to the attachment of two carbohydrate molecules. The term “tri” used in reference to glycosylation refers to the attachment of three carbohydrate molecules. Additionally, the terms “oligo” and “poly” used in reference to a glycosylated molecule refers to the attachment of two or more carbohydrate molecules and can encompass molecules having a variety of attached carbohydrate molecules. As used herein, the terms “sugar,” “sugar moiety,” “sugar molecule,” “saccharide,” “saccharide moiety,” “saccharide molecule,” “carbohydrate,” “carbohydrate moiety,” and “carbohydrate molecule” can be used interchangeably.

As used herein, the term “derivative” refers to a molecule or compound that is derived from a similar compound by some chemical or physical process.

As used herein, the terms “UDP-glycosyltransferase,” “glycosyltransferase,” and “UGT” are used interchangeably to refer to any enzyme capable of transferring sugar residues and derivatives thereof (including but not limited to galactose, xylose, rhamnose, glucose, arabinose, glucuronic acid, and others as understood in the art, e.g., N-acetyl glucosamine) to acceptor molecules. Acceptor molecules, such as melanin precursors, for example, 5,6-DHI, may include other sugars, proteins, lipids, and other organic substrates, such as an alcohol, as disclosed herein. The acceptor molecule can be termed an aglycon (or aglucone, if the sugar is glucose). An aglycon, includes, but is not limited to, the non-carbohydrate part of a glycoside. A “glycoside” as used herein refers an organic molecule with a glycosyl group (organic chemical group derived from a sugar or polysaccharide molecule) connected thereto by way of, for example, an intervening oxygen, nitrogen or sulphur atom. The product of glycosyl transfer can be an O-, N-, S-, or C-glycoside, and the glycoside can be a part of a monosaccharide, disaccharide, oligosaccharide, or polysaccharide. In particular aspects, the glycosyltransferase enzyme is a eukaryotic enzyme, i.e., an enzyme produced in a eukaryotic species including without limitation species from yeast, fungi, plants, and animals. In some embodiments, the glycosyltransferase enzyme is a bacterial enzyme. Examples of UGTs include, but are not limited to, 1 UDP-glucose glycosyltransferases.

Exemplary GenBank Accession Numbers for specific embodiments of such enzymes include: NM_100432.1, NM_113071.2, NM_113073.2, NM_001134258.1, NM_001142488.1, FJ237534.1, GU584127.1, JQ247689.1, NM_059035.1, NM_067587.1, NM_068512.1, NM_072411.1, NM_071915.1, NM_071659.2, NM_071942.2, NM_001028523.1, NM_072419.2, NM_068511.2, NM_001128946.1, NM_001026585.3, NM_059036.5, NM_059037.4, NM_068530.3, NM_001268558.1, NM_070877.3, NM_070897.4, NM_182348.3, NM_071370.3, NM_071577.6, NM_071873.4, NM_071910.3, NM_071916.6, NM_071968.5, NM_071987.4, NM_072409.5, NM_072410.5, NM_072415.3, NM_182344.3, NM_072417.4, NM_001129369.3, NM_075711.5, NM_076781.3, NM_001083287.3, NM_171786.5, GU299097.1, GU299103.1, GU299105.1, GU299107.1, GU299112.1, GU299114.1, GU299116.1, GU299119.1, GU299125.1, GU299126.1, GU299130.1, GU299143.1, NM_001037428.2, AY735003.1, EF408255.1, EF408256.1, NM_001074.2, NM_152404.3, NM_001171873.1, GU170355.1, GU170356.1, GU170357.1, AF093878.1, NM_153314.2, NM_201425.2, NM_201423.2, NM_012683.2, NM_201424.2, NM_001039549.1, NM_057105.3, NM_130407.2, NM_175846.2, NG_005502.3, NM_001039691.2, NG_005503.6, AB499074.1, AB499075.1, AF091397.1, AF091398.1, KC464461.1, JQ247689.1, FJ236328.1, JX011637.1, GU434222.1, GU170357.1, GU170356.1, GU170354.1, GU170355.1, AB541990.1, AB541989.1, EF408256.1, EF408255.1, NM_113073.2, NM_100435.3, NM_113071.2, NM_100432.1, HM543573.1, GU584127.1, AB499075.1, AB499074.1, AAD29570.1, Q06321.1, AAD29571.1 or NM_116337.3.

In particular embodiments, the glycosyltransferase enzyme is Arabidopsis thaliana UGT 71C1, Arabidopsis thaliana UGT 71C1₁₈₈71C2, Arabidopsis thaliana UGT 71C1₂₅₅71C2, Arabidopsis thaliana/Stevia rebaudiana UGT 71C1₂₅₅71E1, Arabidopsis thaliana/Stevia rebaudiana UGT 71C2₂₅₅71E1, Arabidopsis thaliana UGT 71C5, Stevia rebaudiana UGT 71E1, Arabidopsis thaliana UGT 72B1, Arabidopsis thaliana UGT 72B2_L, Arabidopsis thaliana UGT 72B3, Arabidopsis thaliana UGT 72D1, Arabidopsis thaliana UGT 72E2, Stevia rebaudiana UGT 72EV6, Arabidopsis thaliana UGT 73B5, Arabidopsis thaliana UGT 76E12, Arabidopsis thaliana UGT 78D2, Arabidopsis thaliana UGT 89B1, Arabidopsis thaliana UGT 90A2, Rauvolfia serpentina UGT RsAs, Nicotiana tabacum Sa Gtase, or Solanum lycopersicum UGT 74F2.

In particular embodiments, methods provided by the invention using glycosyltransferase are used to glycosylate melanin precursors, derivatives, and/or intermediates in vivo and/or in vitro. Examples of melanin precursors include, but are not limited to, 5,6-DHI, cyclodopa (DHICA), dopachrome, 5,6-dihydroxyindole-2-carboxylic acid, and 6-OH-indole (6-HI). Examples of melanin precursor derivatives comprise other O-methylated molecules, including, but not limited to, 5,6-diacetoxyindole (DAI). Examples of intermediates include, but are not limited to dopaquinone, L-3,4-dihydroxyphenylalanine (L-DOPA), CycloDOPA, dopachrome, 5,6-dihydroxyindole-2-carboxylic acid, and 5,6-DHI.

In another embodiment, glycosylated melanin precursors, derivatives, and/or intermediates may be de-glycosylated using appropriate hydrolase enzymes or alkali treatment.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “about” refers to ±10% of a given value.

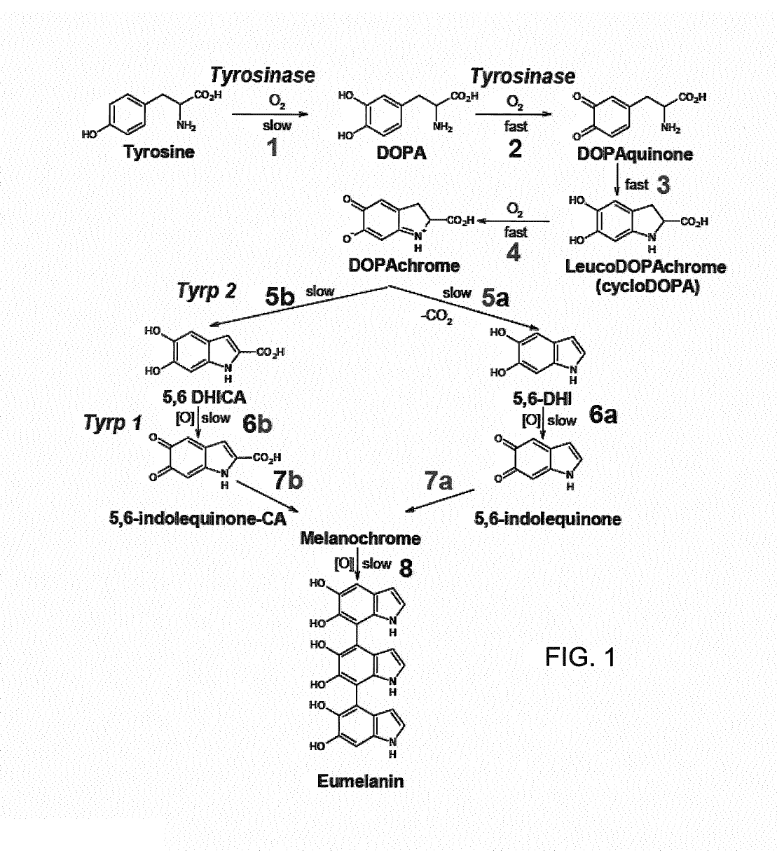

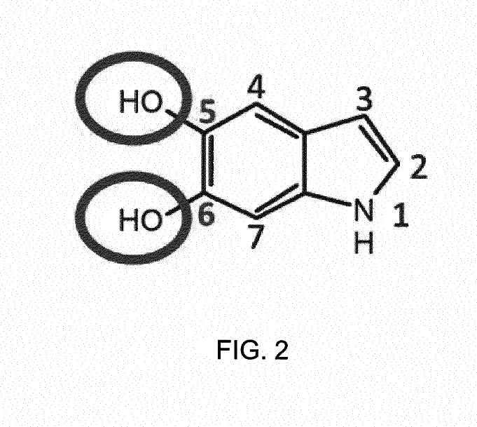

As used herein, the term “melanin precursor” refers to a molecule shown in FIG. 1 including any of L-DOPA, DOPAquinone, LeucoDOPAchrome, DOPAchrome, 5,6-DHICA, 5,6-DHI, 5,6-indolequinone-CA, 5,6-indolequinone, and melanochrome.

As used herein the terms “melanin” or “eumelanin” may be used interchangeably and refer to a polymer of melanochrome.

As used herein, the term “glycosylated melanin” refers to a glycosylated form of melanin.

As used herein, the term “glycosylated melanin precursor” or “GLYMP” refers to a glycosylated form of any melanin precursor. Specific GLYMPs contemplated herein include glycosylated hydroxyindoles, such as mono-glucosylated 5,6-DHI in position 5 (“C1”), mono-glucosylated 5,6-DHI in position 6 (“C2”), and di-glucosylated 5,6-DHI in positions 5 and 6 (“Di-Glc”).

As used herein, the term “pigment” refers to a colored substance produced as a result of a functional melanin biosynthetic pathway being expressed in a recombinant host, and may include 5,6-DHI, eumelanin, pheomelanin, other enzymatic product produced by tyrosinase, and mixtures thereof.

In one embodiment, the present invention contemplates in vivo and in vitro production of melanin, melanin precursors, and glycosylated forms of melanin and melanin precursors. In a further embodiment, the present invention contemplates a combination of in vivo and in vitro steps for the production of melanin, melanin precursors, glycosylated melanin, and/or GLYMPs. In one particular embodiment, the present invention provides recombinant hosts containing an engineered biosynthetic pathway including one or more expressed and functional heterologous enzymes.

For example, the present invention provides recombinant yeast cells capable of producing in vivo melanin precursors. In particular, recombinant yeast cells as provided herein are capable of expressing one or more tyrosinases and/or other proteins capable of converting tyrosine into 5,6-DHI or 5,6-DHICA. Sources for tyrosinases include but are not limited to bacteria, including several species of Rhizobium, Streptomyces, Pseudomonas, and Bacillus that naturally express these enzymes and produce melanin for protection against UV damage and for increased virulence and pathogenesis. In other particular embodiments, tyrosinases used herein can be derived from yeast, fungi, plants, and/or animals.

In another embodiment, recombinant yeast cells capable of expressing one or more tyrosinases and/or other proteins capable of converting tyrosine into 5,6-DHI or 5,6-DHICA are capable of expressing one or more glycosyltransferases that glycosylate 5,6-DHI and/or 5,6-DHICA to form in vivo one or more GLYMPs.

In a further embodiment, recombinant yeast cells capable of expressing one or more glycosyltransferases that can glycosylate 5,6-DHI and/or 5,6-DHICA are cultured in a medium containing 5,6-DHI and/or 5,6-DHICA to form in vivo one or more GLYMPs.

In one embodiment, recombinant cells capable of producing melanin are grown in media enriched with tyrosine to increase melanin precursor production by increasing tyrosine flow into the melanin biosynthetic pathway.

In another embodiment, recombinant cells capable of producing melanin precursors may be further modified to increase melanin precursor production by increasing tyrosine flow into the melanin biosynthetic pathway and/or decreasing the rate of pathway intermediate efflux from the pathway. Similarly, recombinant cells described herein may be modified to emphasize one melanin precursor versus another. For example, as seen in FIG. 1, a recombinant cell may express tyrosinase-related protein 2 (Tyrp2) to shift the equilibrium in favor of 5,6-DHICA versus 5,6-DHI and further express tyrosine-related protein 1 (Tyrp1) to promote melanin formation from DHICA.

### Recombinant Techniques

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

### Functional Homologs

Functional homologs of the polypeptides described herein are also suitable for use in producing melanin precursors and/or GLYMPs in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of melanin biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability as a melanin biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in melanin biosynthesis polypeptides, e.g., conserved functional domains.

Conserved regions can be identified by locating a region within the primary amino acid sequence of a melanin biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing melanin precursors in a recombinant host include functional homologs of tyrosinases and tyrosinase-related proteins. Moreover, polypeptides suitable for producing GLYMPs in a recombinant host include functional homologs of UGTs.

Methods to modify the substrate specificity of, for example, a tyrosinase, tyrosine-related protein, and/or a UGT, are known to those skilled in the art, and include without limitation site-directed/rational mutagenesis approaches, random directed evolution approaches and combinations in which random mutagenesis/saturation techniques are performed near the active site of the enzyme. For example, see Osmani et al., 2009, Phytochemistry 70: 325-347.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A percent (%) identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using the computer program ClustalW (version 1.83, default parameters), which allows alignments of nucleic acid or polypeptide sequences to be carried out across their entire length (global alignment). Chenna et al., 2003, Nucleic Acids Res. 31(13):3497-500.

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignments of nucleic acid sequences, the following parameters are used: gap-opening penalty: 10.0; gap extension penalty: 5.0; and weight transitions: yes. For fast, pairwise, alignment of protein sequences, the following parameters are used: word size: 1; window size: 5; scoring method: % age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine a % identity of a candidate nucleic acid or amino acid sequence to a reference sequence, the sequences are aligned using ClustalW, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

### Protein Variants

It will be appreciated that tyrosinases, tyrosinase-like proteins, and/or UGT proteins can include additional amino acids that are not involved in the enzymatic activities carried out by the enzymes. In some embodiments, tyrosinases, tyrosinase-like proteins, and/or UGT proteins are fusion proteins. The terms “fusion protein” and “chimeric protein” can be used interchangeably refer to proteins engineered through the joining of two or more genes that code for different proteins. In some embodiments, a nucleic acid sequence encoding a tyrosinase, a tyrosinase-like protein, and/or UGT polypeptide can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the protein such that the encoded tag is located at either the carboxyl or amino terminus of the protein. Non-limiting examples of encoded tags include green fluorescent protein (GFP), glutathione S transferase (GST), HIS tag, and Flag™ tag (Kodak, New Haven, Conn.). Other examples of tags include a chloroplast transit peptide, a mitochondrial transit peptide, an amyloplast peptide, signal peptide, or a secretion tag. Such tags may be included in multiples, such as in 6×HIS tags or 3×Flag™ tags or any other desired number or combination.

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

### Regulatory Regions

“Regulatory region” refers to a nucleotide sequence in a given nucleic acid that influences transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element or an upstream activation region (UAR). A regulatory region may be operably linked to a coding sequence by positioning the regulatory region and the coding sequence so that the regulatory region is effective for regulating transcription or translation of the sequence. For example, to link operably a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

### Recombinant Hosts

Recombinant hosts can be used to express polypeptides for producing melanin precursors and GLYMPs, including mammalian, insect, plant, and algal cells. A number of prokaryotes and eukaryotes are also suitable for use in constructing the recombinant microorganisms described herein, e.g., gram-negative bacteria, yeast, and fungi. Genes for which an endogenous counterpart is not present in a particular host strain are advantageously assembled in one or more recombinant constructs, which are then transformed into the strain in order to supply the missing function(s).

The genetically engineered microorganisms provided by the present invention can be cultivated using conventional fermentation processes, including, inter alia, chemostat, batch, fed-batch cultivations, continuous perfusion fermentation, and continuous perfusion cell culture.

Carbon sources of use in the instant method include any molecule that can be metabolized by the recombinant host cell to facilitate growth and/or production of melanin. Examples of suitable carbon sources include, but are not limited to, sucrose (e.g., as found in molasses), fructose, xylose, ethanol, glycerol, glucose, cellulose, starch, cellobiose or other glucose comprising polymer. In embodiments employing yeast as a host, for example, carbon sources such as sucrose, fructose, xylose, ethanol, glycerol, and glucose are suitable. The carbon source can be provided to the host organism throughout the cultivation period or alternatively, the organism can be grown in the presence of another energy source, e.g., protein, and then provided with a source of carbon only during the fed-batch phase.

Exemplary prokaryotic and eukaryotic species are described in more detail below. However, it will be appreciated that other species may be suitable. For example, suitable species can be in a genus such as Agaricus, Aspergillus, Bacillus, Candida, Corynebacterium, Eremothecium, Escherichia, Fusatium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis 32, Rhodoturula mucilaginosa, Phaffia rhodozyma U BV-AX, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia coli, Rhodobacter sphaeroides, Rhodobacter capsulatus, or Rhodotorula toruloides or a eukaryote such as Saccharomyces cerevisiae.

In some embodiments, a microorganism can be an Ascomycete such as Gibberella fujikuroi, Kluyveromyces lactis, Schizosaccharomyces pombe, Aspergillus niger, Yarrowia lipolytica, Ashbya gossypii, or Saccharomyces cerevisiae.

In some embodiments, a microorganism can be an algal cell such as Blakeslea trispora, Dunaliella salina, Haematococcus pluvialis, Chlorella sp., Undaria pinnatifida, Sargassum, Laminaria japonica, or Scenedesmus almeriensis species.

In some embodiments, a microorganism can be a cyanobacterial cell such as Blakeslea trispora, Dunaliella salina, Haematococcus pluvialis, Chlorella sp., Undaria pinnatifida, Sargassum, Laminaria japonica, or Scenedesmus almeriensis.

### Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing rational design of various modules to enhance product yield. Methods are known for making recombinant microorganisms.

### Aspergillus spp.

Aspergillus species such as A. oryzae, A. niger and A. sojae are widely used microorganisms in food production and can be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus. Generally, A. niger is cultured for the industrial production of a number of food ingredients such as citric acid and gluconic acid, and thus species such as A. niger are generally suitable for producing melanin.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing rational design of various modules to enhance product yield. Methods similar to those described above for Saccharomyces can be used to make recombinant E. coli microorganisms.

Agaricus, Gibberella, and Phanerochaete spp. can also be useful.

### Arxula Adeninivorans (Blastobotrys Adeninivorans)

Arxula adeninivorans is a dimorphic yeast (it grows as a budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica.

Yarrowia lipolytica is a dimorphic yeast (see Arxula adeninivorans) and belongs to the family Hemiascomycetes. The entire genome of Yarrowia lipolytica is known. Yarrowia species is aerobic and considered non-pathogenic. Yarrowia is efficient in using hydrophobic substrates (e.g. alkanes, fatty acids, oils) and can grow on sugars. It has a high potential for industrial applications and is an oleaginous microorganism. Yarrowia lipolyptica can accumulate lipid content to approximately 40% of its dry cell weight and is a model organism for lipid accumulation and remobilization. (See e.g., Nicaud, 2012, Yeast 29(10):409-18; Beopoulos et al., 2009, Biohimie 91(6):692-6; Banker et al., 2009, Appl Microbiol Biotechnol. 84(5):847-65).

### Rhodotorula sp.

Rhodotorula is a unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

### Rhodosporidium Toruloides

Rhodosporidium toruloides is an oleaginous yeast and useful for engineering lipid-production pathways (See e.g., Zhu et al., 2013, Nature Commun. 3:1112; Ageitos et al., 2011, Applied Microbiology and Biotechnology 90(4):1219-27).

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

Kluyveromyces lactis

Kluyveromyces lactis is yeast regularly applied to the production of kefir. It can grow on several sugars, most importantly on lactose, which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

Pichia pastoris

Pichia pastoris is methylotrophic yeast (see Candida boidinii and Hansenula polymorpha). It provides an efficient platform for producing foreign proteins. Platform elements are available as a kit, and Pichia pastoris is used worldwide in academia for producing proteins. Strains have been engineered that can produce complex human N-glycan (yeast glycans are similar but not identical to those found in humans). See, e.g., Piirainen et al., 2014, N Biotechnol. 31(6):532-7.

### Physcomitrella spp.

Physcomitrella mosses, when grown in suspension culture, have characteristics similar to yeast or other fungal cultures. This genus can be used for producing plant secondary metabolites, which can be difficult to produce in other types of cells.

### Methods of Producing Melanin Precursors

Recombinant hosts described herein expressing one or more tyrosinase, tyrosinase-like protein, and/or glycosyltransferase genes can be used to produce stable melanin precursors. In one embodiment, non-glycosylated melanin precursors, derivatives, or intermediates can be produced by recombinant hosts, such as, for example, 5,6-DHI.

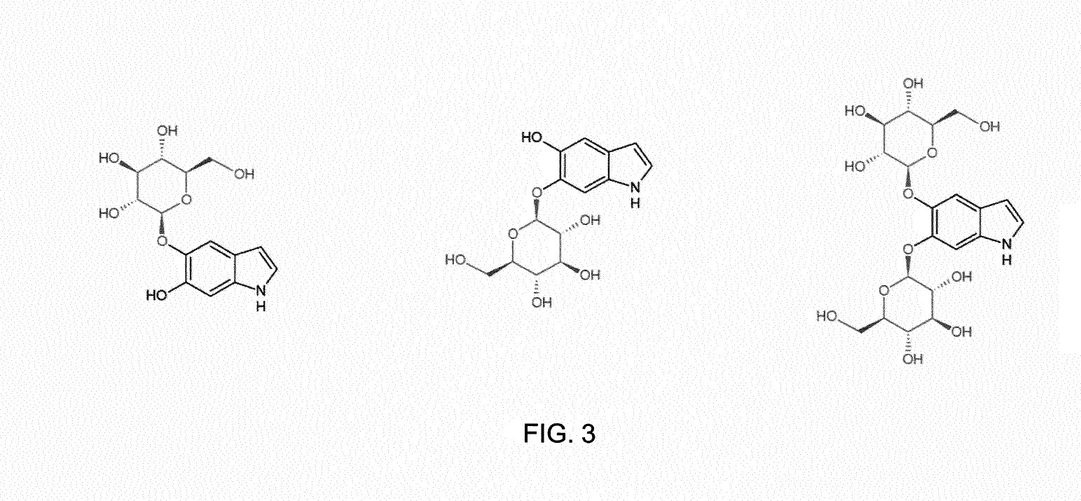

In another embodiment, stable glycosylated melanin precursors can be produced by recombinant hosts (or isolated UGTs in vitro), such as glycosylated forms of 5,6-DHI. In one embodiment, the glycosylated forms of 5,6-DHI can be singly glycosylated forms, such as C1 or C2. In a further embodiment, the glycosylated forms of 5,6-DHI produced can be the double glycosylated form where both of the hydroxyl residues in positions 5 and 6 of 5,6-DHI are glycosylated to form Di-Glc (see FIG. 3).

In one embodiment, a recombinant host or isolated UGT can produce one or more of glycosylated C1, C2, and Di-Glc. For example, a recombinant host or isolated UGT can produce a singly glycosylated form of 5,6-DHI, when the recombinant host expresses a glycosyltransferase with a specific regiospecificity for a particular hydroxyl group, such as position 5 of 5,6-DHI to form C1 or position 6 of 5,6-DHI to form C2. In a further embodiment, glycosyltransferases expressed by the recombinant host can produce two glycosylated forms of 5,6-DHI with specific regiospecificity, such as C1 and C2, or C1 and Di-Glc, or C2 and Di-Glc. In another embodiment, a glycosyltransferase expressed by the recombinant host can produce only Di-Glc or all three glycosylated melanin precursors, C1, C2, and Di-Glc. While not wishing to be bound by theory, it is contemplated that different glycosylated forms of melanin precursors, derivatives, and/or intermediates may be produced by a single glycosyltransferase depending upon whether the reaction occurs in vivo or in vitro.

Methods contemplated herein can include growing a recombinant host in a culture medium under conditions in which melanin biosynthesis and/or glycosyltransferase genes are expressed. The recombinant host can be grown in a fed batch or continuous process. Typically, the recombinant host is grown in a fermentor at a defined temperature(s) for a desired period of time. Depending on the particular host used in the method, other recombinant genes such as tyrosine hydroxylases, p450 or laccases can also be present and may be expressed to produce GLYMPs.

After the recombinant host has been grown in culture for the desired period of time, melanin precursors or GLYMPs can then be recovered (i.e., isolated) from the culture using various techniques known in the art. In some embodiments, a permeabilizing agent can be added to aid the influx of feedstock into the host and product efflux. Further, a crude lysate of the cultured recombinant host can be centrifuged to obtain a supernatant. The resulting supernatant can then be applied to a chromatography column, e.g., a C-18 column, and washed with water to remove hydrophilic compounds followed by elution of the compound(s) of interest with a solvent such as methanol. The compound(s) can then be further purified by preparative HPLC.

It will be appreciated that the various genes discussed herein can be present in two or more recombinant hosts rather than a single host creating plural host system. When such a plurality of recombinant hosts is used, each expressing a piece of the total biosynthetic pathway and none expressing all pieces, they can be grown in a mixed culture to produce the desired products, for example, melanin precursors and/or GLYMPs.

Alternatively, the two or more hosts each can be grown in a separate culture medium and the product of the first culture medium, e.g., 5,6-DHI, can be introduced into second culture medium to be converted into a subsequent intermediate, or into an end product such as, for example, a GLYMP and/or eumelanin (or glycosylated melanin). The product produced by the second, or final host may then be recovered. It will also be appreciated that in some embodiments, a recombinant host may be grown using nutrient sources other than a culture medium and utilizing a system other than a fermentor.

In one embodiment, products and/or pigments produced by the recombinant hosts described herein may be characterized (e.g., identified, quantified, etc.) by measuring absorbance at 500 nm after solubilization in aqueous Soluene® 350 (Perkin Elmer) (see H. Ozeki, et al. Chemical characterization of hair melanins in various coat-color mutants of mice.” J. Invest. Dermatol., vol. 105, no. 3, pp. 361-366, 1995; K. Wakamatsu and S. Ito, “Advanced chemical methods in melanin determination,” Pigment Cell Res., vol. 15, no. 3, pp. 174-183, 2002). This method allows the evaluation of the total amount of melanin contained in the samples. Further, indirect analytical methods may be used based on detection of specific degradation products of 5,6-DHI, 5,6-DHICA, and pheomelanin. Upon alkaline hydrogen peroxide oxidation, pyrrole-2,3-dicarboxylic acid (PDCA) as a specific degradation product of DHI-derived units in eumelanin is formed (see Commo et al. “Age-dependent changes in eumelanin composition in hairs of various ethnic origins,” Int. J. Cosmet. Sci., vol. 34, no. 1, pp. 102-107, 2012; Ito et al. “Chemical Degradation of Melanins: Application to Identification of Dopamine-melanin,” Pigment Cell Res., vol. 11, no. 2, pp. 120-126, 1998). Hydrogen peroxide oxidation also triggers pyrrole-2,3,5-tricarboxylic acid (PTCA) formation as a specific degradation product of DHICA derived units in eumelanin (see Commo et al.; Ito et al, “Microanalysis of eumelanin and pheomelanin in hair and melanomas by chemical degradation and liquid chromatography,” Anal. Biochem., vol. 144, no. 2, pp. 527-536, 1985). The same oxidation in 1 M K₂CO₃ additionally produces thiazole-2,4,5-tricarboxylic acid (TTCA) and thiazole-4,5-dicarboxylic acid (TDCA) as markers for pheomelanin (see Ito et al., “Usefulness of alkaline hydrogen peroxide oxidation to analyze eumelanin and pheomelanin in various tissue samples: Application to chemical analysis of human hair melanins,” Pigment Cell Melanoma Res., vol. 24, no. 4, pp. 605-613, 2011). These degradation products may be separated by HPLC and analyzed with ultraviolet detection.

In another embodiment, products and/or pigments produced by recombinant hosts described herein may be characterized (e.g., identified, quantified, etc.) by liquid NMR of the products and/or pigments dissolved in Soluene® 350 (Perkin Elmer). Another method for characterization of recombinant host products includes ASAP® mass spectrometry, which allows detection of indole-pyrrole units.

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

Recombinant yeast expressing tyrosinases and producing melanin precursors were established. These recombinant yeast cells were subsequently modified to express UGTs also to create strains producing GLYMPs in vivo. Monoglycosylated and diglycosylated GLYMPs were isolated and characterized.

### Example No. 1. Production of Melanin Precursors in Yeast

Eumelanin is present in many organisms in nature, and its production is triggered by enzymes called tyrosinases. Tyrosinases are bifunctional enzymes that can perform both hydroxylation of tyrosine to DOPA and the oxidation of DOPA to DOPAquinone. In this example, S. cerevisiae was transformed with plasmids carrying tyrosinase genes to create melanin precursors/melanin producing strains.

### Methods

Unless otherwise stated, all reagents used herein were purchased from Sigma (St. Louis, Mo.).

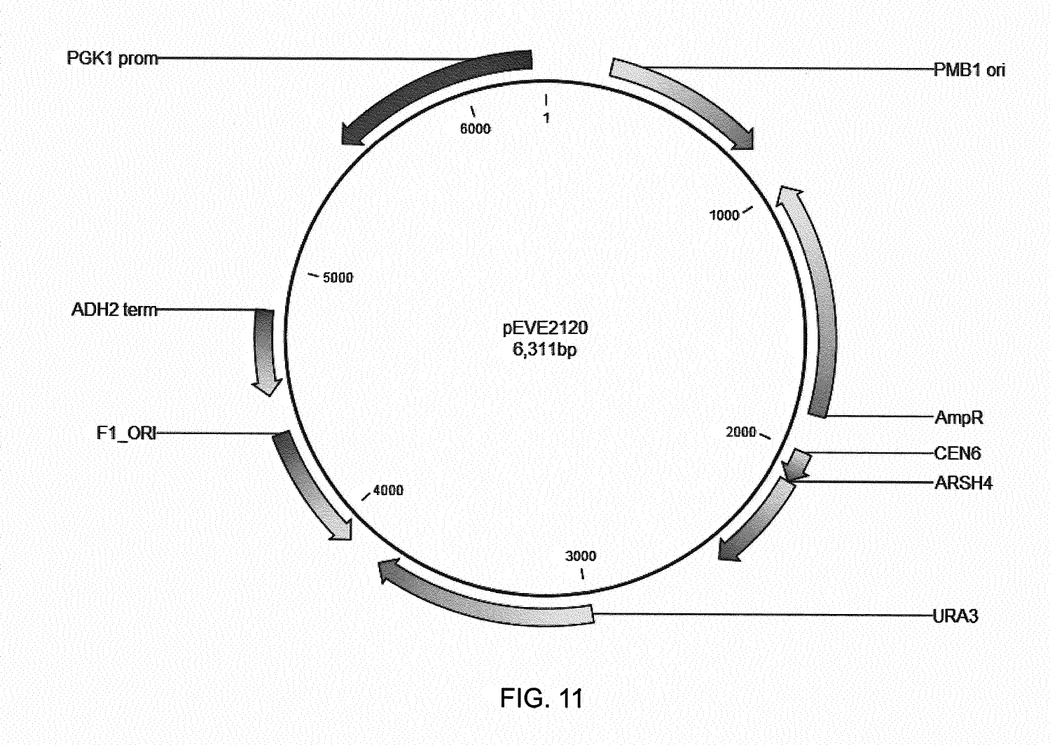

Of twenty-five tyrosinase genes tested, five triggered pigment formation (see Table No. 1) and were codon optimized for S. cerevisiae expression. They were then cloned in yeast expression plasmids (pRS316 modified with the insertion of PGK1 and ADH2 yeast promoter and terminator respectively; see Mumberg et al., Yeast vectors for the controlled expression of heterologous proteins in different genetic backgrounds, Gene 156(1):119-22, 1995) carrying the URA3 auxotrophic marker (see FIG. 11 for plasmid map). Yeast transformation was performed according to conventional methods. See R. D. Gietz and R. Woods, “Yeast Transformation by the LiAc/SS Carrier DNA/PEG Method,” in Yeast Protocol SE—12, vol. 313, W. Xiao, Ed. Humana Press, 2006, pp. 107-120.

TABLE NO. 1
Heterologous Tyrosinases

| Strain | | | Gene SEQ | Protein SEQ |
|---|---|---|---|---|
| ID | ORGANISM | GENE(s) | ID NO | ID NO |
| YN008 | Aspergillus orizae | MELO | 1 | 2 |
| YN013 | Pholiota nameko | TYR-2 | 3 | 4 |
| YN014 | Pycnoporus sanguineus | TYR | 5 | 6 |
| YN075 | Lentinula edodes | TYR | 7 | 8 |
| YN076 | Pholiota nameko | TYR-1 | 9 | 10 |

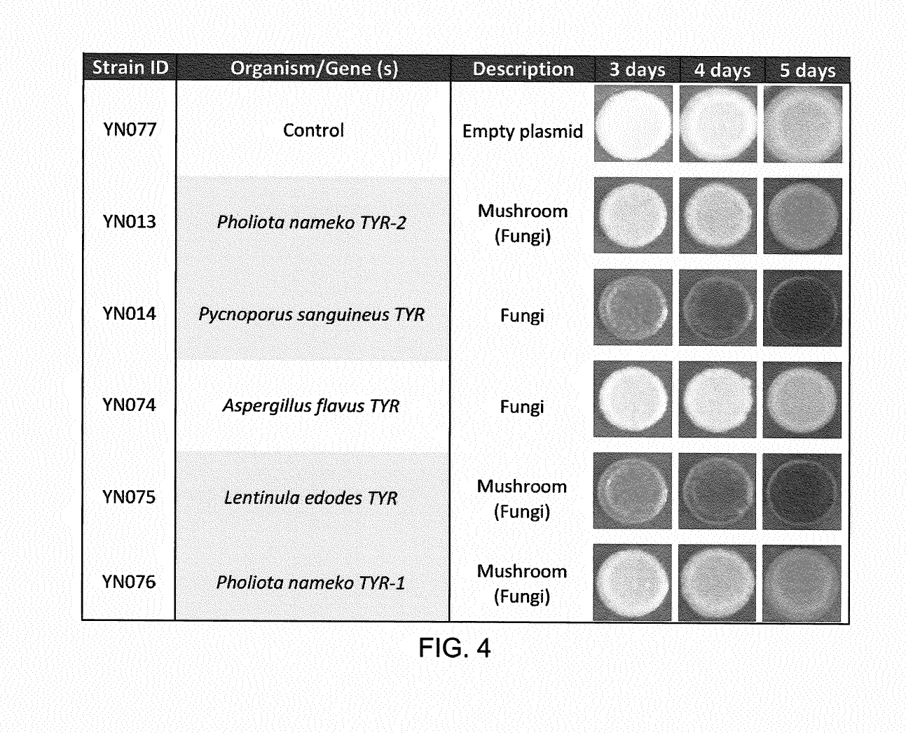

Successfully transformed clones were identified by a clear color change, from white/yellow to brown/black (see FIG. 4).

Yeast clones were tested for color change (from white/yellow to black/brown) to determine which tyrosinase genes could catalyse formation of pigment(s). For each clone, cells were resuspended and serial diluted to a concentration of 10⁴ cells/200 μl H₂O. Eight microliters of the cell suspension were dropped on drop-out SC-agar plates and incubated at 30° C. for 3-5 days to allow accumulation of the pigment(s). The color development of clones was observed during incubation.

### Results

Of the twenty-five tyrosinase gene-containing strains, four were identified (YN013, YN014, YN075, and YN076, identified by SEQ ID NOS: 4, 6, 8, and 10, respectively) as being able to trigger pigment(s) formation in yeast (see FIG. 4). These results demonstrate the establishment of a functional, heterologous melanin biosynthetic pathway in recombinant yeast cells.

### Example No. 2. Enhanced Formation of Pigment(s) in Yeast Fed Tyrosine

In this example, pigment(s) formation was increased in recombinant S. cerevisiae strains from Example No. 1 provided with increased exogenous tyrosine.

A strategy for increasing production of a certain compound in yeast is to increase intracellular pathway precursor levels. The biological pathway for eumelanin production is triggered by the conversion of tyrosine into DOPA (see FIG. 1), and thus increased levels of tyrosine could boost eumelanin formation in yeast. Tyrosine is a non-essential amino acid and is been naturally produced by yeast cells, and additionally, it can be taken up from the surrounding growth medium thanks to specialized transporters present on the plasma membrane. See V. Sophianopoulou and G. Diallinas, “Amino acid transporters of lower eukaryotes: Regulation, structure and topogenesis,” FEMS Microbiol. Rev., vol. 16, no. 1, pp. 53-75, 1995; F. Omura, H. Hatanaka, and Y. Nakao, “Characterization of a novel tyrosine permease of lager brewing yeast shared by Saccharomyces cerevisiae strain RM11-1a,” FEMS Yeast Res., vol. 7, no. 8, pp. 1350-1361, 2007). Therefore, increased levels of tyrosine were used to test whether tyrosine supplementation of the growth medium could increase pigment production in the tyrosinase-transformed clones.

### Methods of Tyrosine Supplementation

Synthetic complete (SC) media contain 0.42 mM tyrosine. Additional tyrosine was added to both media to reach a final concentration of 1.42 mM. For agar plates: cells were resuspended and serial diluted to a concentration of 10⁴ cells/200 μl H₂O. Eight microliters of the cell suspension were dropped on drop-out SC-agar plates supplemented with 1.42 mM tyrosine. Plates were incubated at 30° C. for 5 days to allow accumulation of the pigment(s). For liquid media: strains were grown in standard media for 16 h to saturation and diluted to OD₆₀₀=0.1 in media supplemented with 1.42 mM tyrosine. Cultures were incubated for 3 days.

### Results

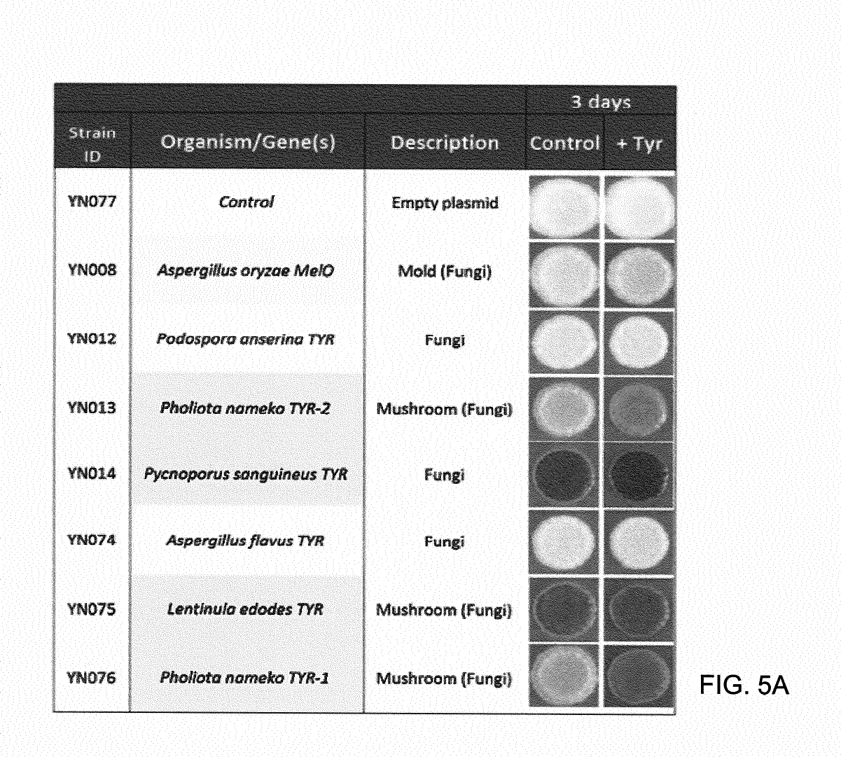

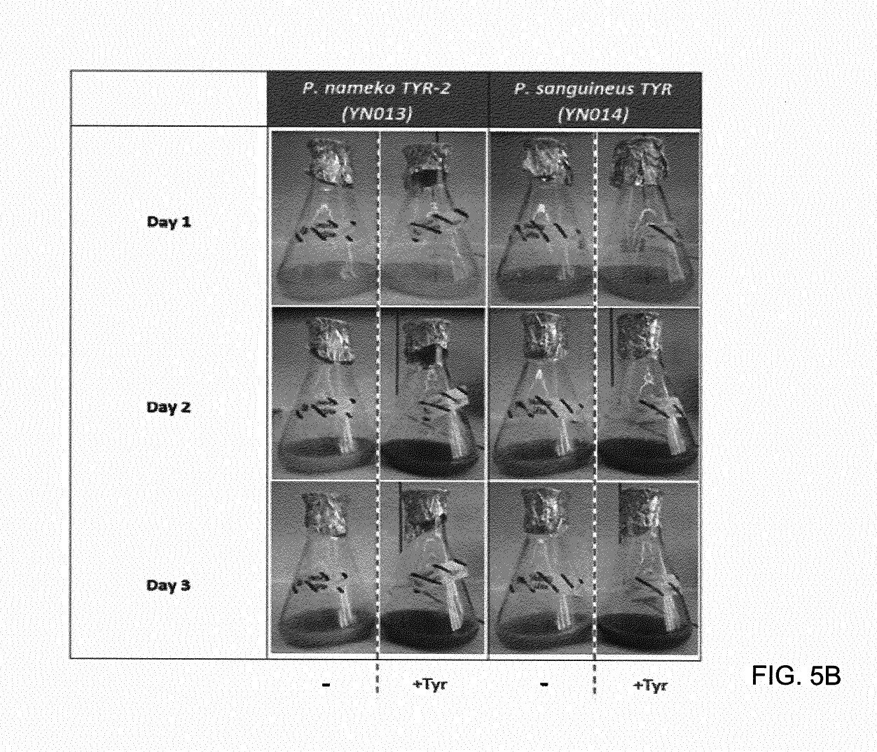

Strains containing tyrosinases able to trigger pigment(s) formation showed an increase in browning with an increased tyrosine concentration in the media. These results were seen whether growing cells either on agar plates (FIG. 5A) or in liquid media (FIG. 5B). Furthermore, in the presence of increased tyrosine levels, the strain YN008, containing the MelO tyrosinase from A. orizae (SEQ ID NO: 2), which did not show any browning using standard SC medium, showed a slight browning after 3 days of incubation (FIG. 5A). Therefore, these results demonstrate that pigment(s) production levels in recombinant yeast may be increased by tyrosine supplementation.

### Example No. 3. Identification of UGTs Able to Glycosylate 5,6-DHI In Vitro

UGTs transformed into a melanin-producing yeast strain may be able to slow or stop spontaneous polymerization of melanin precursors by the formation of Glycosylated Melanin Precursors (GLYMPs). Therefore, in this example, UGTs able to glycosylate the melanin precursor 5,6-DHI to form GLYMPs were sought via in vitro screening.

A collection of in vitro purified UGT enzymes from plants was utilized for a high throughput (HT) screening for the identification of enzymes able to transfer sugar moiety(ies) to 5,6-DHI, supplied UDP-glucose as a sugar donor.

### Methods

In Vitro Glycosylation Reaction

A pool of 50 μL reactions was prepared mixing the following components:

Enzymes:

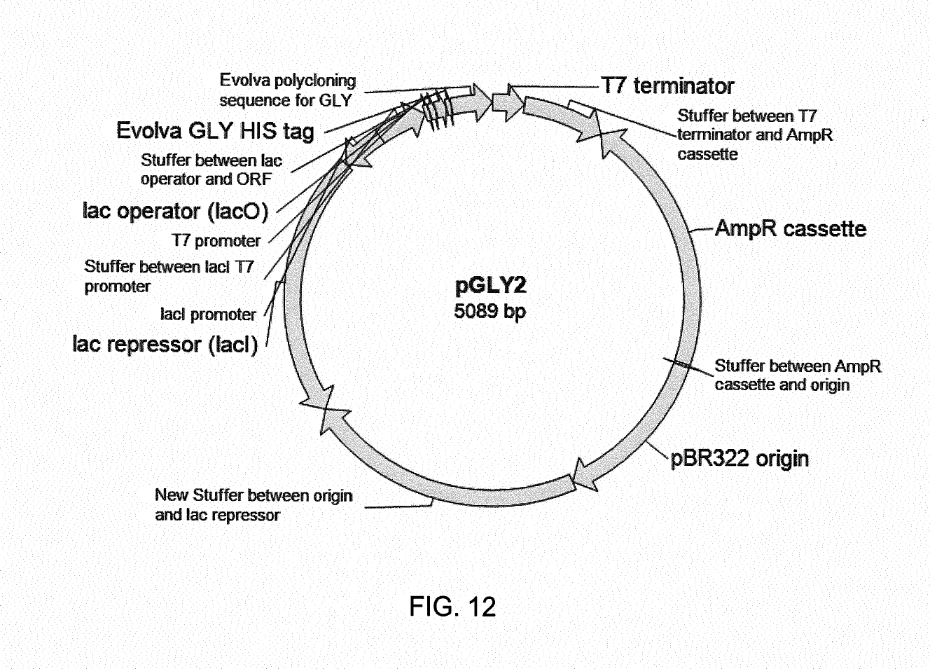

UGT genes were cloned in an appropriate E. coli expression vector (synthesized by “GeneArt™ gene synthesis,” see FIG. 12) and were transformed and expressed in an E. coli system (100 mL cultures), purified via conventional methods, and eluted in 300 μL elution buffer (via 6×His-tag purification, see Hochuli et al., Genetic Approach to Facilitate Purification of Recombinant Proteins with a Novel Metal Chelate Adsorbent, Nature Biotechnology, November 1988, pages 1321-1325). Since there was no direct correlation between enzyme concentration and its activity, a fixed volume of enzyme preparations was added to each reaction (5 μL).

Sugar Donor:

UDP-sugar was added to each reaction to reach a final concentration of 0.6 mM.

Reaction Buffer:

100 mM Tris-base, 5 mM MgCl₂, 1 mM KCl, pH 8.0.

Substrate:

5,6-DHI dissolved in DMSO was added to each reaction to reach a final concentration of 0.2 mM (3:1 molar ratio to sugar donor: 5,6-DHI). Reactions were incubated overnight at 30° C. with mild shaking and directly injected for LC-MS analysis.

Glymps Analysis:

An analytical method for GLYMPs analysis was developed on a Waters® UPLC (Ultra Performance Liquid Chromatography) system equipped with a Waters® 2777 sample manager, and a PDA detector. The system was also coupled to a Waters® SQD (Single Quadrupole) mass spectrometer.

Column:

BEH Acquity C18, 2.1×100 mm, 1.7 μm particle size (Part no. 186002352). The column was kept at 35° C. for the duration of the run. Mobile phases: A: Deionized water+0.1% Formic Acid; B: Acetonitrile+0.1% Formic Acid. The gradient is shown in Table No. 2. Flow rate: 0.4 mL/min.

TABLE NO. 2
UPLC mobile phase gradient.

| Time (min) | % B |
|---|---|
| 0 | 1 |
| 5 | 50 |
| 5.5 | 100 |
| 7 | 100 |
| 7.1 | 1 |
| 10 | 1 |

Mass Spectrometry Conditions:

ESI-Single ion recording (SIR) 310 Da; capillary 3.4 kV, cone 30V, extraction 3V, RF Lens 0.1V; source temp 150° C., desolvation temp 350° C.; desolvation gas 450 L/hr, cone gas 50 L/hr. Samples were identified by accurate mass analysis.

### Results

Of 262 UGTs tested, twenty-one catalyzed formation of GLYMPs (both monoglucosylated (in position 5 or 6) and di-glucosylated (in both positions 5 and 6)). The successful UGTs are listed in Table No. 3.

TABLE NO. 3
UGTs for 5,6-DHI glucosylation.

| | | | Gene | Protein |
|---|---|---|---|---|
| Plasmid | | | SEQ | SEQ |
| ID | Organism | UGT | ID NO: | ID NO: |
| pG103 | Arabidopsis thaliana | 71C1 | 11 | 12 |
| pG191 | Arabidopsis thaliana | 71C1₁₈₈71C2 | 13 | 14 |
| pG185 | Arabidopsis thaliana | 71C1₂₅₅71C2 | 15 | 16 |
| pG187 | Arabidopsis thaliana/ | 71C1₂₅₅71E1 | 17 | 18 |
| | Stevia rebaudiana | | | |
| pG183 | Arabidopsis thaliana/ | 71C2₂₅₅71E1 | 19 | 20 |
| | Stevia rebaudiana | | | |
| pG104 | Arabidopsis thaliana | 71C5 | 21 | 22 |
| pG132 | Stevia rebaudiana | 71E1 | 23 | 24 |
| pG135 | Arabidopsis thaliana | 72B1 | 25 | 26 |
| pG136 | Arabidopsis thaliana | 72B2_l | 27 | 28 |
| pG106 | Arabidopsis thaliana | 72B3 | 29 | 30 |
| pG042 | Arabidopsis thaliana | 72D1 | 31 | 32 |
| pG155 | Arabidopsis thaliana | 72E2 | 33 | 34 |
| pG188 | Stevia rebaudiana | 72EV6 | 35 | 36 |
| pG137 | Arabidopsis thaliana | 73B5 | 37 | 38 |
| pG098 | Arabidopsis thaliana | 76E12 | 39 | 40 |
| pG112 | Arabidopsis thaliana | 78D2 | 41 | 42 |
| pG079 | Arabidopsis thaliana | 89B1 | 43 | 44 |
| pG149 | Arabidopsis thaliana | 90A2 | 45 | 46 |
| pG021 | Rauvolfia serpentina | RsAs | 47 | 48 |
| pG184 | Nicotiana tabacum | SA Gtase | 49 | 50 |
| pG186 | Solanum lycopersicum | 74F2 | 51 | 52 |

HT screening results are shown in Table No. 4 below.

TABLE NO. 4
HT screening results.

| | | | | |
|---|---|---|---|---|
| Plasmid | | | | Relative protein |
| ID | UGT name | Peak area | Retention Time | concentration |

Mono-glycosylated 5,6-DHI (Position 5)

| | | | | |
|---|---|---|---|---|
| pG188 | 72EV6 | 258875 | 2.18 | 102.2 |
| pG135 | 72B1 | 212117 | 2.19 | 164.8 |
| pG079 | 89B1 | 181037 | 2.17 | 84.7 |
| pG042 | 72D1 | 132551 | 2.18 | 189.9 |
| pG187 | 71C1₂₅₅71E1 | 40275 | 2.17 | 110.9 |
| pG183 | 71C2₂₅₅71E1 | 32225 | 2.15 | 95.1 |
| pG103 | 71C1 | 18599 | 2.17 | 171.4 |
| pG104 | 71C5 | 6017 | 2.18 | 9.1 |
| pG021 | AS | 2192 | 2.16 | BLQ |
| pG136 | 72B2_L | 1968 | 2.16 | BLQ |
| pG184 | SA Gtase | 1725 | 2.18 | 52.9 |
| pG191 | 71C1₁₈₈71C2 | 1582 | 2.16 | 15.7 |
| pG155 | 72E2 | 1551 | 2.15 | 169.1 |
| pG185 | 71C1₂₅₅71C2 | 1386 | 2.13 | 29.2 |
| pG106 | 72B3 | 1378 | 2.17 | 6.9 |
| pG137 | 73B5 | 1352 | 2.15 | 288.2 |

Mono-glycosylated 5,6-DHI (Position 6)

| | | | | |
|---|---|---|---|---|
| pG079 | 89B1 | 372434 | 2.46 | 84.7 |
| pG187 | 71C1₂₅₅71E1 | 109832 | 2.46 | 110.9 |
| pG042 | 72D1 | 62054 | 2.45 | 189.9 |
| pG184 | SA Gtase | 53685 | 2.46 | 52.9 |
| pG183 | 71C2₂₅₅71E1 | 17834 | 2.45 | 95.1 |
| pG103 | 71C1 | 6520 | 2.45 | 171.4 |
| pG188 | 72EV6 | 6039 | 2.48 | 102.2 |
| pG149 | 90A2 | 4998 | 2.45 | 156 |
| pG186 | 74F2 | 4054 | 2.48 | 55.9 |
| pG136 | 72B2_L | 3451 | 2.46 | BLQ |
| pG185 | 71C1₂₅₅71C2 | 2103 | 2.43 | 29.2 |
| pG098 | 76E12 | 1519 | 2.45 | 258.2 |
| pG191 | 71C1₁₈₈71C2 | 1482 | 2.45 | 15.7 |
| pG137 | 73B5 | 1468 | 2.43 | 288.2 |
| pG132 | 71E1 | 1331 | 2.45 | BLQ |

Di-glycosylated 5,6-DHI (Positions 5 and 6)

| | | | | |
|---|---|---|---|---|
| pG132 | 71E1 | 344803 | 2.06 | BLQ |
| pG187 | 71C1₂₅₅71E1 | 142710 | 2.03 | 110.9 |
| pG112 | 78D2 | 10167 | 2.01 | 72.4 |
| pG079 | 89B1 | 5024 | 2.01 | 84.7 |

Relative protein concentration: Calculated as percentage of 1 μg standard BSA loaded on SDS gel. BLQ: below the limit of quantitation.

The results shown in Table No. 4 demonstrate that certain UGTs can glycosylate one or both positions 5 and 6 of 5,6-DHI and with different efficiencies. As a further assessment of candidate UGTs ability to glycosylate 5,6-DHI, UGTs 89B1 (SEQ ID NO: 44) and 71C1₂₅₅71E1 (SEQ ID NO: 18) were chosen for an in vitro production of small amounts of mono- and di-glucosylated 5,6-DHI, and the compound structures were confirmed by NMR analysis (data not shown). Cumulatively, these results indicate that in vitro and/or combined in vivo/in vitro production of GLYMPs can provide a useful source of glycosylated melanin precursors.

### Example No. 4. Formation of GLYMPs in Yeast Fed with the Melanin Precursor 5,6-DHI

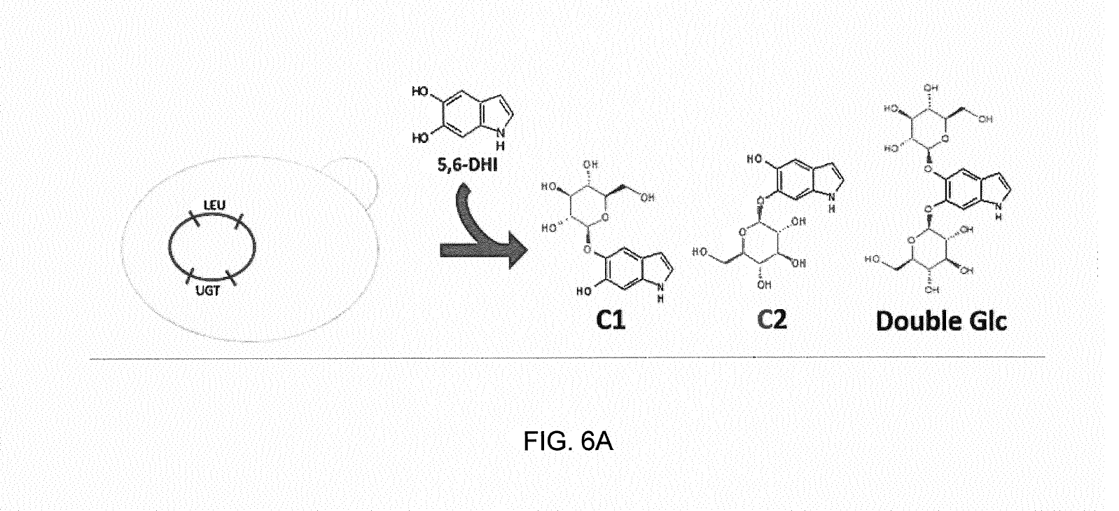

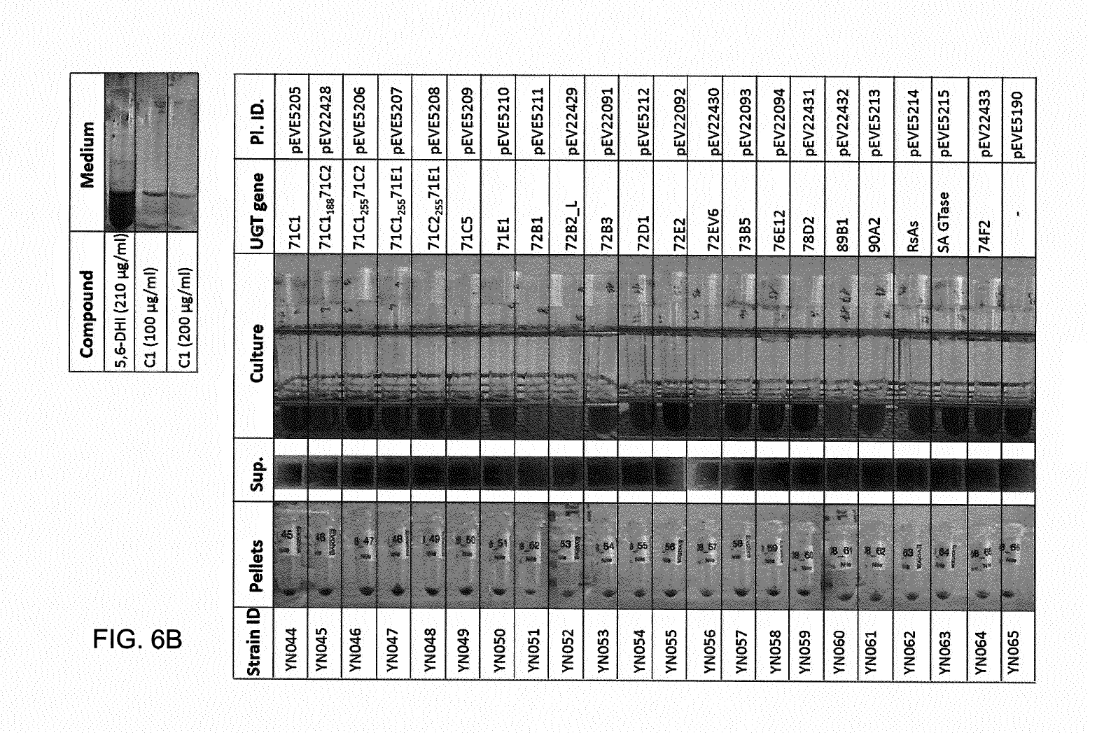

In this example, GLYMPs formation was characterized in S. cerevisiae strains containing heterologous UGT genes only, provided with the exogenous melanin precursor 5,6-DHI. A pictorial representation of the experiment is shown in FIG. 6A.

### Methods

Growth of Yeast Cultures for 5,6-DHI Feeding

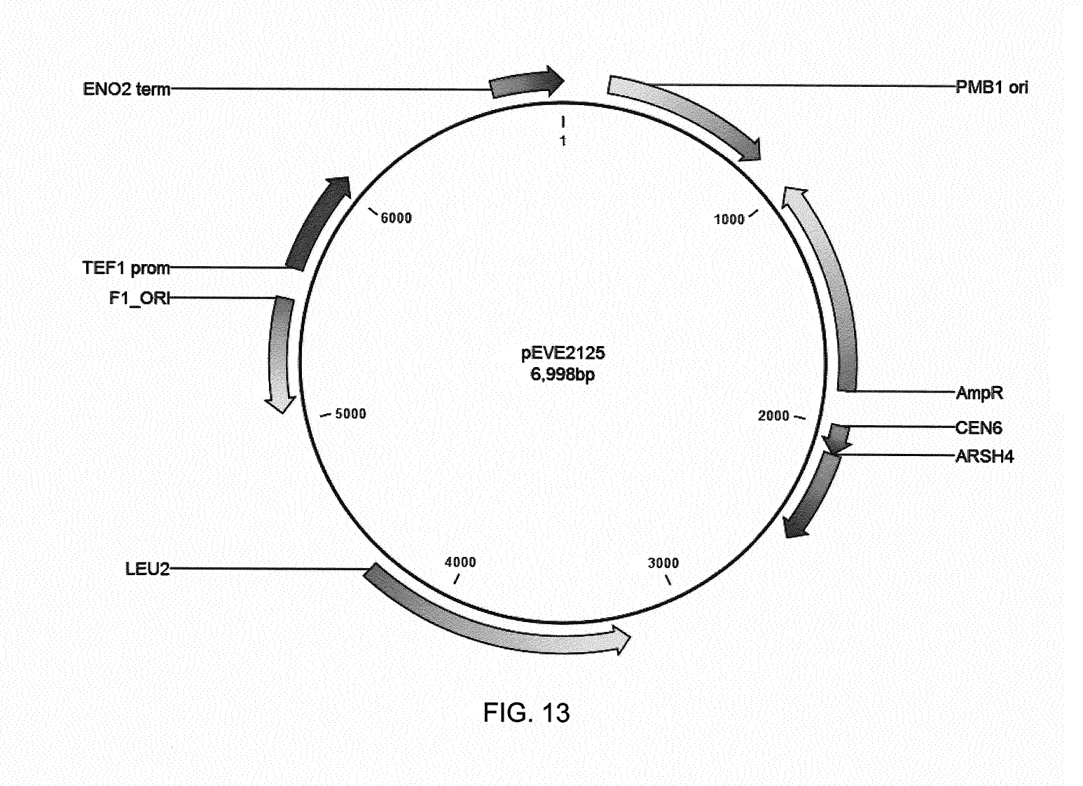

The UGT genes identified via the HT screening (Example No. 3) were cloned in yeast expression vectors (see Mumberg et al., Yeast vectors for the controlled expression of heterologous proteins in different genetic backgrounds, Gene 156(1):119-22, 1995) based on pRS315 and modified with the insertion of a yeast TEF1 promoter, a yeast ENO2 terminator, and a LEU2 auxotrophic marker (see FIG. 13). The plasmids were then transformed in S. cerevisiae cells. The yeast cells obtained thereby were grown overnight at 30° C. in appropriate drop out medium. After 18 h, cultures were diluted to ˜OD₆₀₀ 0.05 in 50 mL medium. Cells were grown to ˜OD₆₀₀=0.5, and 5,6-DHI was added to a final concentration of 210 mg/L. Cells were harvested at ˜OD₆₀₀=1.

Analytical Method for the Detection of In Vivo Generated GLYMPs

GLYMPs Extraction from Yeast Cells

GLYMPs were extracted from yeast cells according to the following protocol:

A sample of 50 mL of culture was centrifuged at 4,000 rpm for 10 min to separate cells (pellet) and growth medium. An aliquot of 500 μL of ddH₂O was added to the pellet, and the cells were resuspended and transferred into 2 mL Eppendorf® screw caps tubes. Five hundred microliters of glass beads were added, and cells were lysed by 3 cycles in a Precellys® 24 cell homogenizer (Bertin Technologies, Rockville, Md.) (60 sec cycles, 6,000 rpm, 40 sec break between cycles).

Lysed cells were clearified by centrifugation at 14,000 rpm for 3 min, and 600 μL of the supernatants were loaded on conditioned SPE cartridges (sample pre-cleaning). The columns were initially washed with 1 mL 5% MeOH. Sample elution was performed with 2 rounds of 1 mL 95% MeOH washes. Eluates were collected in V-shaped glass tubes, and the samples were evaporated for 2 hr in a Lyo Speed Genevac® HT-4× (Genevac Ltd, Ipswich, UK).

An aliquot of 200 μL of ddH₂O was then added to the dried samples, and the resulting mixtures were briefly sonicated (ca. 10 sec) to dissolve the material. The dissolved samples were transferred into HPLC vials with 300 μL glass inserts and centrifuged for 5 min at 5,000 rpm. Samples of 5 μL of the clear supernatant were injected over LC-MS along with a calibration curve 3-1000 ng/ml.

Analytical Method for the Detection of In Vivo Generated GLYMPs

An analytical method for detection of in vivo generated GLYMPs was developed on a Waters® UPLC (Ultra Performance Liquid Chromatography) system equipped with a Waters® 2777 sample manager, and a PDA detector. The system was also coupled to a Waters® SQD (Single Quadrupole) mass spectrometer.

Column:

BEH Acquity C18, 2.1×100 mm, 1.7 μm particle size (Part no. 186002352). The column was kept at 35° C. for the duration of the run. Mobile phases: A: Deionized water+0.1% Formic Acid. B: Acetonitrile+0.1% Formic Acid. The gradient is shown in Table No. 5. Flow rate: 0.4 mL/min.

TABLE NO. 5
UPLC mobile phase gradient.

| Time (min) | % B |
|---|---|
| 0 | 1 |
| 5 | 50 |
| 5.5 | 100 |
| 7 | 100 |
| 7.1 | 1 |
| 10 | 1 |

Mass Spectrometry Conditions:

ESI-Single ion recording (SIR) 310 Da; capillary 3.4 kV, cone 30V, extraction 3V, RF Lens 0.1V; source temp 150° C., desolvation temp 350° C.; desolvation gas 450 L/hr, cone gas 50 L/hr.

Standards:

C1, C2, and double glycosylated 5,6-DHI produced in vitro and validated by NMR analysis (see Example No. 3) were utilized as standard compounds for the identification and quantification of the in vivo produced GLYMPs. Five microliters of the purified compound at a concentration of 500 ng/mL were injected.

### Results

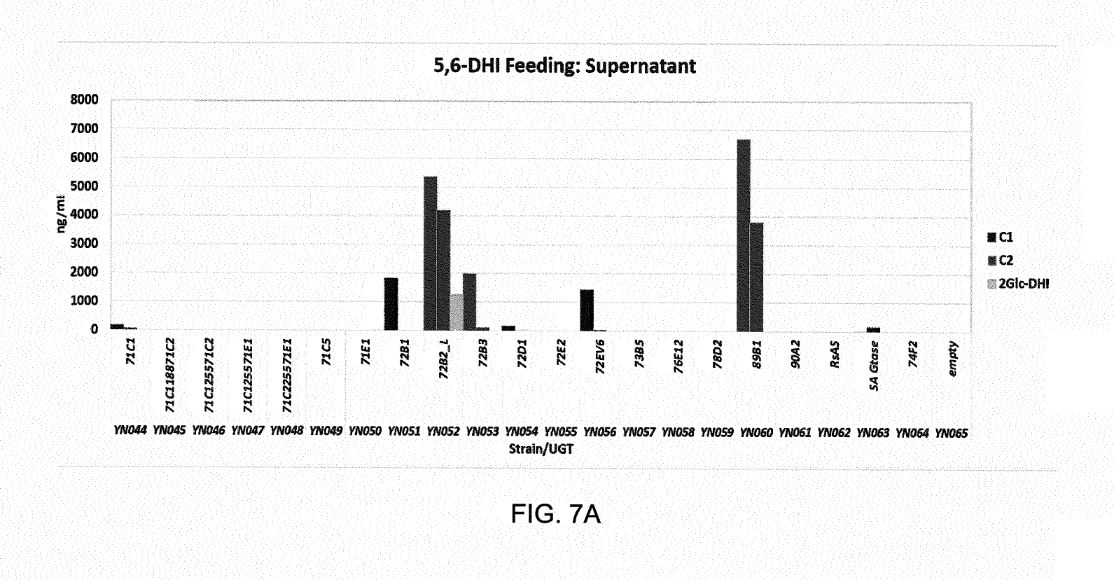

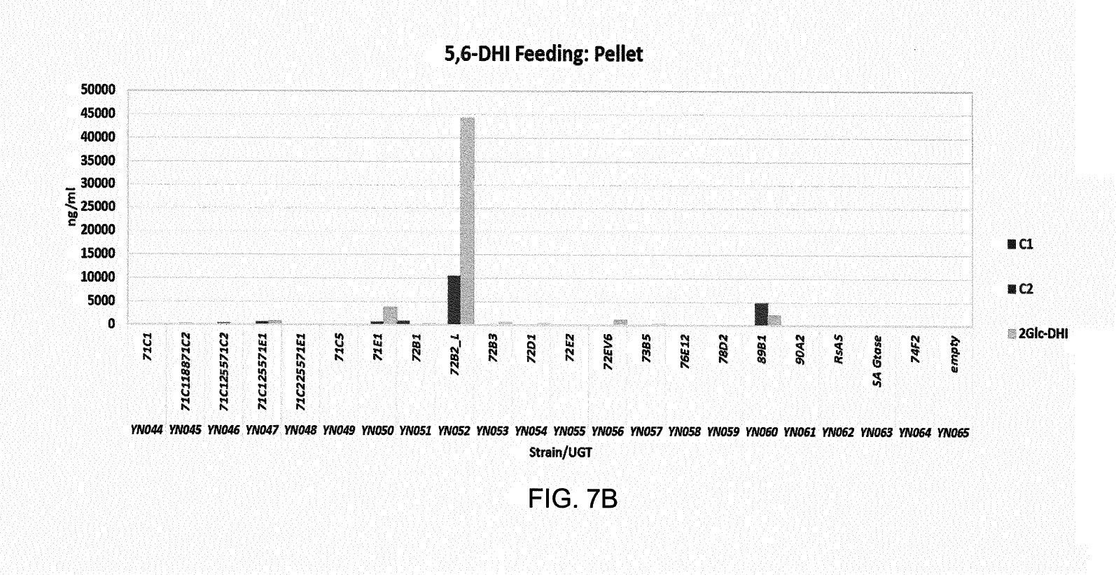

Samples of the cultures grown for the 5,6-DHI feeding experiment, together with the obtained pellets and supernatants after centrifugation, are shown in FIG. 6B. Cultures showed varied colors, ranging from black to yellow. Those cultures where GLYMPs formation was detected showed a color closer to yellow rather than black. GLYMPs were detected in both extracted supernatants (FIG. 7A) and pellets (FIG. 7B). UGTs 71E1 (SEQ ID NO: 24), 72B1 (SEQ ID NO: 26), 72B2_L (SEQ ID NO: 28), 72B3 (SEQ ID NO: 29), 72D1 (SEQ ID NO:32), 72EV6 (SEQ ID NO:36), 89B1 (SEQ ID NO: 44), and SA Gtase (SEQ ID NO: 50), which produced GLYMPs upon 5,6-DHI feeding, were selected for the in vivo experiment described in Example No. 5.

### Example No. 5. In Vivo Production of GLYMPs in Yeast

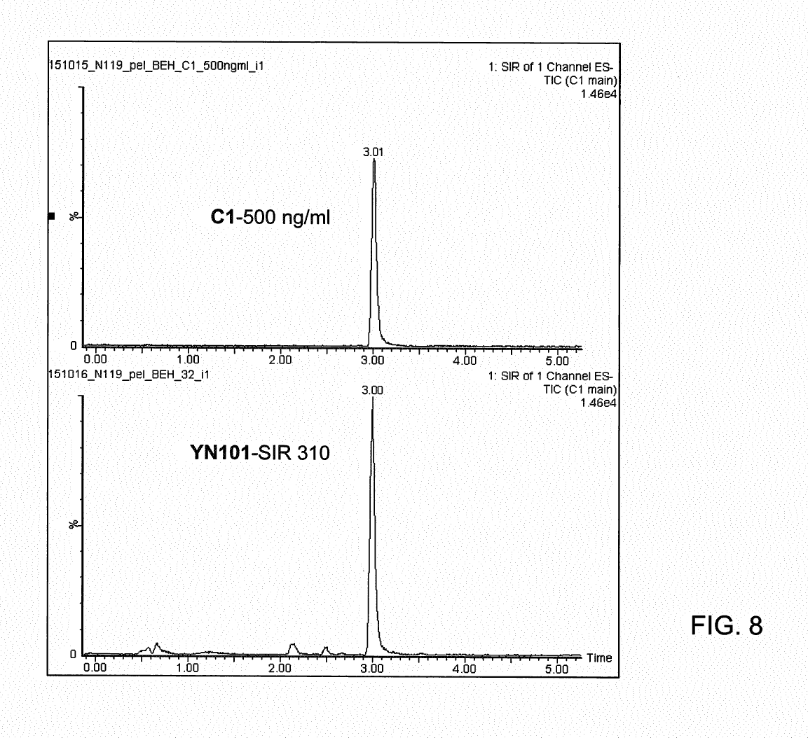

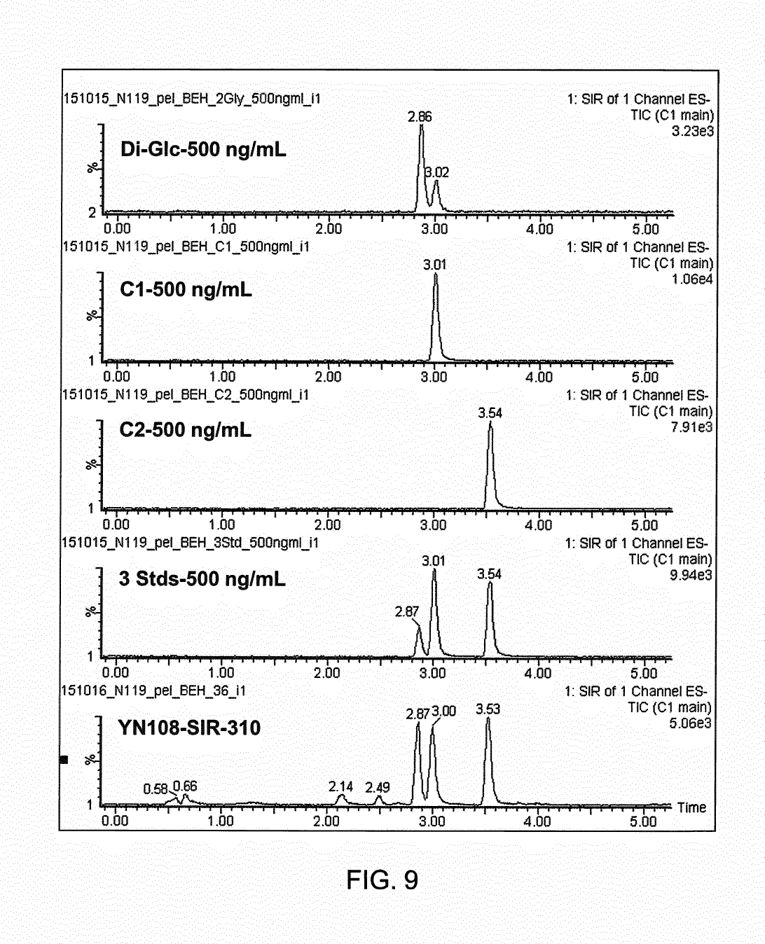

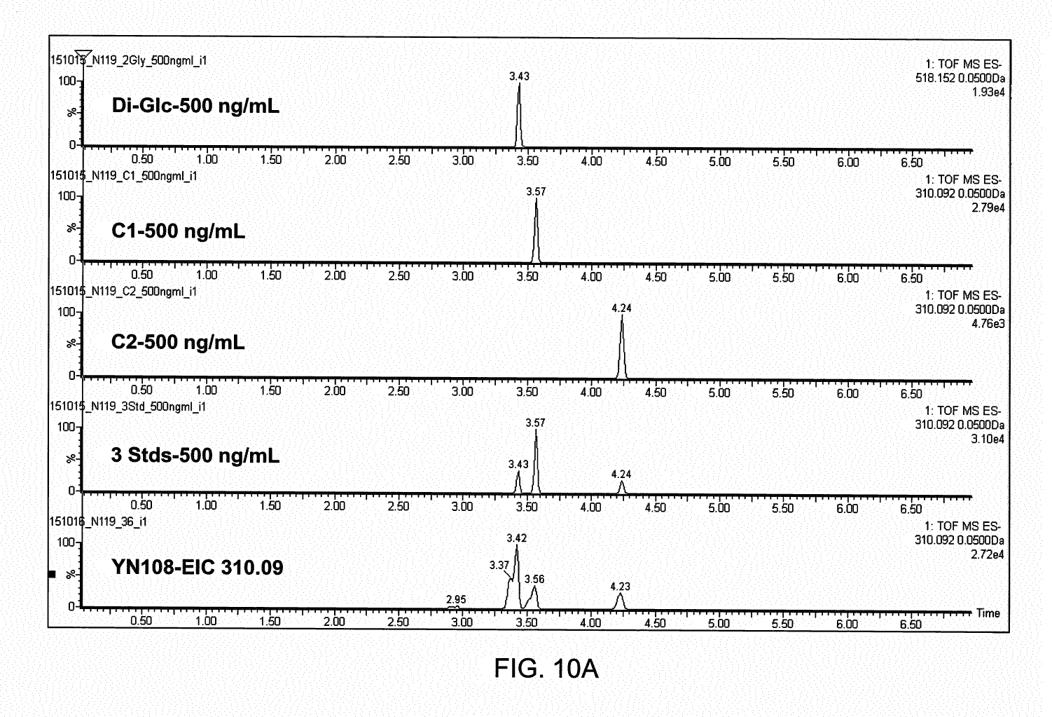

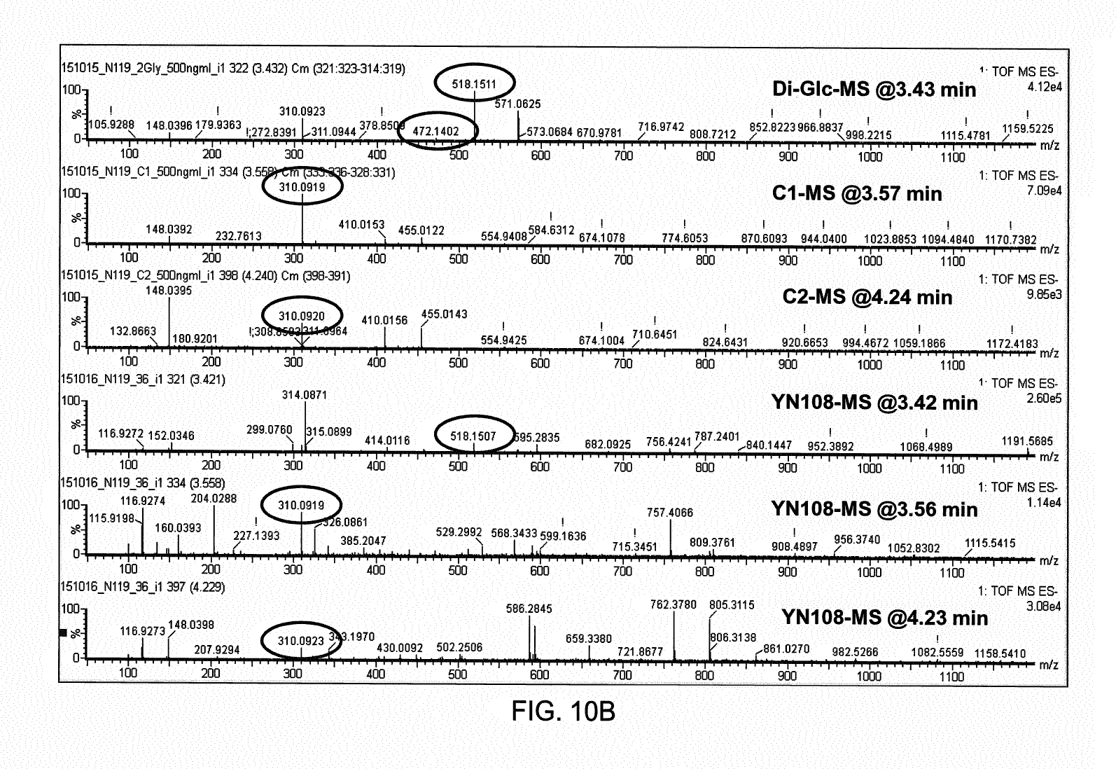

In this example, UGTs identified in Example No. 4 were co-expressed in Saccharomyces cerevisiae with the tyrosinases identified in Example Nos. 1-2. GLYMPs formation was confirmed by LC-MS and TOF analysis (for strains YN101 and YN108, see FIGS. 8-10B).

Methods

UGTs 71E1 (SEQ ID NO: 24), 72B1 (SEQ ID NO: 26), 72B2_L (SEQ ID NO: 28), 72B3 (SEQ ID NO: 29), 72D1 (SEQ ID NO:32), 72EV6 (SEQ ID NO:36), 89B1 (SEQ ID NO: 44), and SA Gtase (SEQ ID NO: 50) cloned in yeast expression vectors (see above) were co-transformed with the five tyrosinase genes that triggered pigment(s) formation (described in Example Nos. 1 and 2).

GLYMPs were extracted and analyzed by LC-MS according to the method reported in Example No. 4.

TOF analysis: Column used: BEH Acquity C18, 2.1×100 mm, 1.7 μm particle size (Part no. 186002352). The column was kept at 30° C. Mobile phases: A: Deionized water+0.1% Formic Acid. B: Acetonitrile+0.1% Formic Acid. The gradient is shown in Table No. 6. Flow: 0.4 ml/min.

TABLE NO. 6
UPLC mobile phase gradient.

| Time (min) | % B |
|---|---|
| 0 | 1 |
| 7 | 20 |
| 7.1 | 100 |
| 8 | 100 |
| 8.1 | 1 |
| 10 | 1 |

Mass spectrometry conditions: Instrument: Waters® Xevo G2-XS QTof. Acquisition time 0-10 min. SN: YEA617. Source: ESI−. Polarity: Negative. Analyzer Mode: Sensitivity. Dynamic range Extended. Target Enhancement: Off. Mass range 50-1,200 Da. Scan Time 0.3 sec. Data Format: Centroid. Capillary 1 kV, Cone 40 V, Source offset 80 V. Source temperature 150° C., Desolvation temperature 500° C. Desolvation gas 100 L/hr, Cone gas 1000 L/hr.

### Results

Plasmids carrying the five tyrosinase genes inducing pigment(s) formation (Example Nos. 1 and 2) and those carrying the UGTs identified in Example No. 4 were co-expressed (see Table No. 7). Several conditions were screened: temperature of incubation (24-30° C.), time of incubation (24-48 hr), presence of additional tyrosine in the growth medium (0.42-1.42 mM). The couples of genes reported in Table No. 7 triggered the formation of the indicated GLYMPs.

TABLE NO. 7
in vivo GLYMPs formation strains.

| | | SEQ | | SEQ | |
|---|---|---|---|---|---|
| Strain | | ID | | ID | |
| ID | Tyrosinase | NO: | UGT | NO: | GLYMP(s) |
| YN029 | P. sanguineus TYR | 6 | 71E1 | 24 | C1, C2, di-glc |
| YN030 | P. sanguineus TYR | 6 | 72B1 | 26 | C1 |
| YN031 | P. sanguineus TYR | 6 | 72B2_L | 28 | C1, C2, di-glc |
| YN033 | P. sanguineus TYR | 6 | 72D1 | 32 | di-glc |
| YN035 | P. sanguineus TYR | 6 | 72EV6 | 36 | C1 |
| YN039 | P. sanguineus TYR | 6 | 89B1 | 44 | C1 |
| YN143 | A. orizae MELO | 2 | 71E1 | 24 | C2, di-glc |
| YN144 | A. orizae MELO | 2 | 72B1 | 26 | C1 |
| YN145 | A. orizae MELO | 2 | 72B2_L | 28 | C1, C2, di-glc |
| YN146 | A. orizae MELO | 2 | 72D1 | 32 | C1, C2 |
| YN147 | A. orizae MELO | 2 | 72EV6 | 36 | C1 |
| YN148 | A. orizae MELO | 2 | 89B1 | 44 | C1, C2 |
| YN094 | P. nameko TYR2 | 4 | 71E1 | 24 | di-glc |
| YN095 | P. nameko TYR2 | 4 | 72B1 | 26 | C1 |
| YN096 | P. nameko TYR2 | 4 | 72B2_L | 28 | C1, C2, di-glc |
| YN097 | P. nameko TYR2 | 4 | 72D1 | 32 | di-glc |
| YN098 | P. nameko TYR2 | 4 | 89B1 | 44 | C1 |
| YN100 | L. edodes TYR | 8 | 71E1 | 24 | di-glc |
| YN101 | L. edodes TYR | 8 | 72B1 | 26 | C1 |
| YN102 | L. edodes TYR | 8 | 72B2_L | 28 | C1, C2, di-glc |
| YN103 | L. edodes TYR | 8 | 72D1 | 32 | di-glc |
| YN104 | L. edodes TYR | 8 | 89B1 | 44 | C1, C2 |
| YN106 | P. nameko TYR1 | 10 | 71E1 | 24 | di-glc |
| YN107 | P. nameko TYR1 | 10 | 72B1 | 26 | C1 |
| YN108 | P. nameko TYR1 | 10 | 72B2_L | 28 | C1, C2, di-glc |
| YN110 | P. nameko TYR1 | 10 | 89B1 | 44 | C1, C2 |

GLYMPs were detected in extracted yeast pellets. The LC-MS analyses on products from strains YN101 and YN108, as well as TOF analysis, is reported in FIGS. 8-10B.

Sequence Identities

| | |
|---|---|
| SEQ ID NO: 1 | Aspergillus orizae MELO, ORF codon |
| | optimized for S. cerevisiae |
| SEQ ID NO: 2 | Amino acid sequence encoded by the nucleic |
| | acid encoded by SEQ ID NO: 1 |
| SEQ ID NO: 3 | Pholiota nameko TYR-2, ORF codon |
| | optimized for S. cerevisiae |
| SEQ ID NO: 4 | Amino acid sequence encoded by the nucleic |
| | acid encoded by SEQ ID NO: 3 |
| SEQ ID NO: 5 | Pycnoporus sanguineus tyrosinase, ORF |
| | codon optimized for S. cerevisiae |
| SEQ ID NO: 6 | Amino acid sequence encoded by the nucleic |
| | acid encoded by SEQ ID NO: 5 |
| SEQ ID NO: 7 | Lentinula edodes tyrosinase, ORF codon |
| | optimized for S. cerevisiae |
| SEQ ID NO: 8 | Amino acid sequence encoded by the nucleic |
| | acid encoded by SEQ ID NO: 7 |
| SEQ ID NO: 9 | Pholiota nameko TYR-1 tyrosinase, ORF |
| | codon optimized for S. cerevisiae |
| SEQ ID NO: 10 | Amino acid sequence encoded by the nucleic |
| | acid encoded by SEQ ID NO: 9 |
| SEQ ID NO: 11 | Arabidopsis thaliana UGT 71C1 |
| SEQ ID NO: 12 | Amino acid sequence encoded by the nucleic |
| | acid encoded by SEQ ID NO: 11 |
| SEQ ID NO: 13 | Arabidopsis thaliana UGT 71C1₁₈₈71C2 |
| SEQ ID NO: 14 | Amino acid sequence encoded by the nucleic |
| | acid encoded by SEQ ID NO: 13 |
| SEQ ID NO: 15 | Arabidopsis thaliana UGT 71C1₂₅₅71C2 |
| SEQ ID NO: 16 | Amino acid sequence encoded by the nucleic |
| | acid encoded by SEQ ID NO: 15 |
| SEQ ID NO: 17 | Arabidopsis thaliana/Stevia rebaudiana UGT |
| | 71C1₂₅₅71E1 |
| SEQ ID NO: 18 | Amino acid sequence encoded by the nucleic |
| | acid encoded by SEQ ID NO: 17 |
| SEQ ID NO: 19 | Arabidopsis thaliana/Stevia rebaudiana UGT |
| | 71C2₂₅₅71E1 |
| SEQ ID NO: 20 | Amino acid sequence encoded by the nucleic |
| | acid encoded by SEQ ID NO: 19 |
| SEQ ID NO: 21 | Arabidopsis thaliana UGT 71C5 |
| SEQ ID NO: 22 | Amino acid sequence encoded by the nucleic |
| | acid encoded by SEQ ID NO: 21 |
| SEQ ID NO: 23 | Stevia rebaudiana UGT 71E1 |
| SEQ ID NO: 24 | Amino acid sequence encoded by the nucleic |
| | acid encoded by SEQ ID NO: 23 |
| SEQ ID NO: 25 | Arabidopsis thaliana UGT 72B1 |
| SEQ ID NO: 26 | Amino acid sequence encoded by the nucleic |
| | acid encoded by SEQ ID NO: 25 |
| SEQ ID NO: 27 | Arabidopsis thaliana UGT 72B2_L |
| SEQ ID NO: 28 | Amino acid sequence encoded by the nucleic |
| | acid encoded by SEQ ID NO: 27 |
| SEQ ID NO: 29 | Arabidopsis thaliana UGT 72B3 |
| SEQ ID NO: 30 | Amino acid sequence encoded by the nucleic |
| | acid encoded by SEQ ID NO: 29 |
| SEQ ID NO: 31 | Arabidopsis thaliana UGT 72D1 |
| SEQ ID NO: 32 | Amino acid sequence encoded by the nucleic |
| | acid encoded by SEQ ID NO: 31 |
| SEQ ID NO: 33 | Arabidopsis thaliana UGT 72E2 |
| SEQ ID NO: 34 | Amino acid sequence encoded by the nucleic |
| | acid encoded by SEQ ID NO: 33 |
| SEQ ID NO: 35 | Stevia rebaudiana UGT 72EV6 |
| SEQ ID NO: 36 | Amino acid sequence encoded by the nucleic |
| | acid encoded by SEQ ID NO: 35 |
| SEQ ID NO: 37 | Arabidopsis thaliana UGT 73B5 |
| SEQ ID NO: 38 | Amino acid sequence encoded by the nucleic |
| | acid encoded by SEQ ID NO: 37 |
| SEQ ID NO: 39 | Arabidopsis thaliana UGT 76E12 |
| SEQ ID NO: 40 | Amino acid sequence encoded by the nucleic |
| | acid encoded by SEQ ID NO: 39 |
| SEQ ID NO: 41 | Arabidopsis thaliana UGT 78D2 |
| SEQ ID NO: 42 | Amino acid sequence encoded by the nucleic |
| | acid encoded by SEQ ID NO: 41 |
| SEQ ID NO: 43 | Arabidopsis thaliana UGT 89B1 |
| SEQ ID NO: 44 | Amino acid sequence encoded by the nucleic |
| | acid encoded by SEQ ID NO: 43 |
| SEQ ID NO: 45 | Arabidopsis thaliana UGT 90A2 |
| SEQ ID NO: 46 | Amino acid sequence encoded by the nucleic |
| | acid encoded by SEQ ID NO: 45 |
| SEQ ID NO: 47 | Rauvolfia serpentina UGT RsAs |
| SEQ ID NO: 48 | Amino acid sequence encoded by the nucleic |
| | acid encoded by SEQ ID NO: 47 |
| SEQ ID NO: 49 | Nicotiana tabacum Sa Gtase |
| SEQ ID NO: 50 | Amino acid sequence encoded by the nucleic |
| | acid encoded by SEQ ID NO: 49 |
| SEQ ID NO: 51 | Solanum lycopersicum UGT 74F2 |
| SEQ ID NO: 52 | Amino acid sequence encoded by the nucleic |
| | acid encoded by SEQ ID NO: 51 |
| SEQ ID NO: 53 | pG187 expression vector |

Sequences

| | |
|---|---|
| SEQ ID NO: 1 | ATGGCCTCTGTCGAACCTATTAAGACCTTCGAAATTAGACAAAAGGGTCCAGTTGAAACTA |
| | AGGCCGAAAGAAAGTCTATCAGAGACTTGAACGAAGAAGAATTGGACAAGTTGATTGAA |
| | GCCTGGAGATGGATTCAAGATCCAGCTAGAACTGGTGAAGATTCCTTTTTTTACTTGGCCG |
| | GTTTACATGGTGAACCTTTTAGAGGTGCTGGTTACAACAATTCTCATTGGTGGGGTGGTTA |
| | TTGTCATCATGGTAACATTTTGTTCCCAACCTGGCATAGAGCTTATTTGATGGCTGTTGAAA |
| | AGGCTTTGAGAAAAGCCTGTCCAGATGTTTCTTTGCCATATTGGGATGAATCTGATGACGA |
| | AACTGCTAAGAAAGGTATCCCATTGATCTTCACCCAAAAAGAATACAAGGGTAAGCCAAA |
| | CCCATTATACTCTTACACCTTCTCCGAAAGAATCGTTGATAGATTGGCTAAGTTTCCAGATG |
| | CCGATTACTCTAAACCACAAGGTTACAAGACTTGCAGATATCCATATTCTGGTTTGTGCGGT |
| | CAAGATGATATTGCTATTGCTCAACAACACAACAATTTCTTGGACGCCAATTTCAATCAAGA |
| | ACAAATCACCGGTTTGTTGAACTCCAATGTTACTTCTTGGTTGAACTTGGGTCAATTCACCG |
| | ATATTGAAGGTAAGCAAGTTAAGGCTGATACCAGATGGAAGATTAGACAATGTTTGTTGA |
| | CCGAAGAATACACCGTTTTCTCTAACACTACTTCTGCTCAAAGATGGAACGATGAACAATTC |
| | CATCCATTGGAATCTGGTGGTAAAGAAACTGAAGCTAAGGCTACTTCTTTGGCTGTTCCAT |
| | TAGAATCTCCACATAACGATATGCATTTGGCCATTGGTGGTGTTCAAATTCCAGGTTTTAAC |
| | GTTGATCAATACGCTGGTGCTAATGGTGATATGGGTGAAAATGATACTGCTTCCTTCGATC |
| | CAATCTTCTACTTTCATCATTGCTTCATCGACTACTTGTTCTGGACTTGGCAAACCATGCATA |
| | AGAAAACTGATGCCTCCCAAATTACCATCTTGCCAGAATATCCAGGTACAAACTCTGTTGAT |
| | TCTCAAGGTCCAACTCCAGGTATTTCTGGTAATACTTGGTTGACTTTGGATACCCCATTGGA |
| | TCCATTCAGAGAAAATGGTGACAAAGTCACCTCTAACAAGTTGTTGACCTTGAAGGATTTG |
| | CCATACACTTACAAAGCTCCAACTTCTGGTACTGGTTCTGTTTTTAATGATGTCCCAAGATT |
| | GAACTACCCATTGTCTCCACCAATTTTGAGAGTTTCCGGTATTAACAGAGCTTCCATTGCTG |
| | GTTCTTTTGCCTTGGCTATTTCACAAACTGATCATACTGGTAAGGCTCAAGTCAAGGGTATT |
| | GAATCTGTTTTGTCTAGATGGCATGTTCAAGGTTGTGCTAACTGTCAAACTCATTTGTCTAC |
| | TACTGCTTTCGTCCCTTTGTTCGAATTGAATGAAGATGACGCCAAGAGAAAGCACGCTAAC |
| | AATGAATTAGCTGTTCACTTGCATACCAGAGGTAATCCAGGTGGTCAAAGAGTTAGAAAC |
| | GTTACTGTTGGTACTATGAGATAA |
| SEQ ID NO: 2 | MASVEPIKTFEIRQKGPVETKAERKSIRDLNEEELDKLIEAWRWIQDPARTGEDSFFYLAGLHGE |
| | PFRGAGYNNSHWWGGYCHHGNILFPTWHRAYLMAVEKALRKACPDVSLPYWDESDDETAK |
| | KGIPLIFTQKEYKGKPNPLYSYTFSERIVDRLAKFPDADYSKPQGYKTCRYPYSGLCGQDDIAIAQ |
| | QHNNFLDANFNQEQITGLLNSNVTSWLNLGQFTDIEGKQVKADTRWKIRQCLLTEEYTVFSNT |
| | TSAQRWNDEQFHPLESGGKETEAKATSLAVPLESPHNDMHLAIGGVQIPGFNVDQYAGANG |
| | DMGENDTASFDPIFYFHHCFIDYLFWTWQTMHKKTDASQITILPEYPGTNSVDSQGPTPGISG |
| | NTWLTLDTPLDPFRENGDKVTSNKLLTLKDLPYTYKAPTSGTGSVFNDVPRLNYPLSPPILRVSG |
| | INRASIAGSFALAISQTDHTGKAQVKGIESVLSRWHVQGCANCQTHLSTTAFVPLFELNEDDAK |
| | RKHANNELAVHLHTRGNPGGQRVRNVTVGTMR |
| SEQ ID NO: 3 | ATGTCCAGAGTTGTTATCACCGGTGTTTCTGGTACTGTTGCTAATAGATTGGAAATCAACG |
| | ACTTCGTCAAGAACGACAAGTTCTTCTCATTGTACATTCAAGCCTTGCAAGTCATGTCATCT |
| | GTTCCACCACAAGAAAACGTTAGATCCTTCTTTCAAATCGGTGGTATTCATGGTTTGCCATA |
| | TACTCCATGGGATGGTATTACTGGTGATCAACCATTTGATCCAAATACTCAATGGGGTGGT |
| | TACTGTACTCATGGTTCTGTTTTGTTTCCAACTTGGCATAGACCATACGTCTTGTTGTATGAA |
| | CAAATCTTGCACAAGCACGTTCAAGATATTGCTGCTACTTATACCACTTCTGATAAGGCTGC |
| | TTGGGTTCAAGCTGCTGCTAATTTGAGACAACCATATTGGGATTGGGCTGCTAATGCTGTT |
| | CCTCCAGATCAAGTTATTGCTTCTAAGAAGGTTACCATCACTGGTTCTAATGGTCACAAGGT |
| | TGAAGTTGACAACCCATTATACCATTACAAGTTCCACCCAATCGATTCCTCATTTCCAAGAC |
| | CATATTCTGAATGGCCAACTACCTTAAGACAACCTAATTCTTCTAGACCAAACGCCACTGAT |
| | AATGTCGCTAAGTTGAGAAATGTTTTGAGAGCTTCCCAAGAAAACATCACCTCTAACACTT |
| | ACTCTATGTTGACCAGAGTTCATACTTGGAAGGCTTTCTCTAATCATACTGTTGGTGATGGT |
| | GGTTCTACCTCTAATTCTTTGGAAGCTATTCATGATGGTATCCACGTTGATGTAGGTGGTG |
| | GTGGTCATATGGCTGATCCAGCTGTTGCTGCTTTTGATCCTATTTTCTTCTTGCATCACTGCA |
| | ACGTCGACAGATTATTGTCTTTGTGGGCAGCTATTAACCCAGGTGTTTGGGTTTCTCCAGG |
| | TGATTCTGAAGATGGTACTTTCATTTTGCCACCTGAAGCTCCAGTTGATGTTTCTACTCCATT |
| | AACTCCATTCTCTAACACCGAAACTACTTTTTGGGCTTCTGGTGGTATTACAGATACAACTA |
| | AGTTGGGTTACACCTACCCAGAATTCAATGGTTTGGATTTGGGTAATGCTCAAGCTGTTAA |
| | GGCTGCAATTGGTAACATCGTTAACAGATTATACGGTGCCTCTGTTTTTTCTGGTTTTGCTG |
| | CTGCAACTTCTGCTATTGGTGCTGGTTCAGTTGCTTCTTTGGCTGCTGATGTTCCATTGGAA |
| | AAAGCTCCAGCTCCTGCTCCAGAAGCTGCCGCTCAATCTCCAGTTCCAGCACCAGCTCATGT |
| | TGAACCAGCTGTTAGAGCTGTTTCTGTTCATGCTGCAGCTGCTCAACCACATGCTGAACCA |
| | CCAGTTCACGTTTCTGCCGGTGGTCATCCATCTCCACATGGTTTTTATGATTGGACCGCTAG |
| | AATCGAATTCAAGAAGTACGAATTCGGTTCCTCCTTTTCCGTTTTGTTGTTTTTGGGTCCAG |
| | TTCCTGAAGATCCAGAACAATGGTTAGTTTCTCCAAATTTCGTTGGTGCTCATCATGCTTTT |
| | GTTAATTCTGCTGCTGGTCATTGTGCTAACTGTAGAAATCAAGGTAACGTTGTTGTTGAAG |
| | GTTTCGTTCATTTGACCAAGTACATTTCTGAACATGCCGGTTTGAGATCTTTGAACCCAGAA |
| | GTTGTTGAACCTTACTTGACCAACGAATTGCATTGGAGAGTTTTGAAAGCTGATGGTAGTG |
| | TTGGTCAATTGGAATCCTTGGAAGTTTCTGTTTATGGTACTCCAATGAACTTGCCAGTTGGT |
| | GCTATGTTTCCTGTTCCAGGTAATAGAAGACATTTCCATGGTATCACTCACGGTAGAGTTG |
| | GTGGTAGTAGACATGCTATAGTTTAA |
| SEQ ID NO: 4 | MSRVVITGVSGTVANRLEINDFVKNDKFFSLYIQALQVMSSVPPQENVRSFFQIGGIHGLPYTP |
| | WDGITGDQPFDPNTQWGGYCTHGSVLFPTVVHRPYVLLYEQILHKHVQDIAATYTTSDKAAW |
| | VQAAANLRQPYWDWAANAVPPDQVIASKKVTITGSNGHKVEVDNPLYHYKFHPIDSSFPRPY |
| | SEWPTTLRQPNSSRPNATDNVAKLRNVLRASQENITSNTYSMLTRVHTWKAFSNHTVGDGG |
| | STSNSLEAIHDGIHVDVGGGGHMADPAVAAFDPIFFLHHCNVDRLLSLWAAINPGVWVSPGD |
| | SEDGTFILPPEAPVDVSTPLTPFSNTETTFWASGGITDTTKLGYTYPEFNGLDLGNAQAVKAAIG |
| | NIVNRLYGASVFSGFAAATSAIGAGSVASLAADVPLEKAPAPAPEAAAQSPVPAPAHVEPAVR |
| | AVSVHAAAAQPHAEPPVHVSAGGHPSPHGFYDWTARIEFKKYEFGSSFSVLLFLGPVPEDPEQ |
| | WLVSPNFVGAHHAFVNSAAGHCANCRNQGNVVVEGFVHLTKYISEHAGLRSLNPEVVEPYLT |
| | NELHWRVLKADGSVGQLESLEVSVYGTPMNLPVGAMFPVPGNRRHFHGITHGRVGGSRHAI |
| | V |
| SEQ ID NO: 5 | ATGTCCCACTTCATCGTTACTGGTCCAGTTGGTGGTCAAACTGAAGGTGCTCCAGCTCCAA |
| | ATAGATTGGAAATCAACGATTTCGTCAAGAACGAAGAATTTTTCTCATTATACGTTCAAGCC |
| | TTGGACATCATGTACGGTTTGAAACAAGAAGAATTGATCTCCTTCTTCCAAATCGGTGGTA |
| | TTCATGGTTTGCCATATGTTGCTTGGTCTGATGCTGGTGCTGATGATCCAGCTGAACCATCT |
| | GGTTACTGTACTCATGGTTCTGTTTTGTTTCCAACTTGGCATAGACCATACGTTGCCTTGTAT |
| | GAACAAATCTTGCATAAGTACGCTGGTGAAATTGCTGATAAGTACACTGTTGATAAGCCAA |
| | GATGGCAAAAAGCTGCTGCTGATTTGAGACAACCATTTTGGGATTGGGCTAAGAATACTTT |
| | GCCACCACCAGAAGTTATTTCTTTGGATAAGGTTACTATCACCACCCCAGATGGTCAAAGA |
| | ACTCAAGTTGATAATCCATTGAGAAGATACAGATTCCACCCAATCGATCCATCTTTTCCAGA |
| | ACCATATTCTAATTGGCCAGCTACTTTGAGACATCCAACATCTGATGGTTCTGATGCTAAGG |
| | ATAACGTTAAGGATTTGACTACTACCTTGAAGGCTGATCAACCAGATATTACTACTAAGAC |
| | CTACAACTTGTTGACCAGAGTTCATACTTGGCCAGCCTTTTCTAATCATACTCCAGGTGATG |
| | GTGGTTCCTCTTCTAATTCTTTGGAAGCCATTCATGATCACATCCACGATTCTGTAGGTGGT |
| | GGTGGTCAAATGGGTGATCCATCTGTTGCTGGTTTTGATCCAATTTTCTTCTTGCATCATTG |
| | CCAAGTCGATAGATTATTGGCTTTGTGGTCTGCTTTGAATCCAGGTGTTTGGGTTAATTCCT |
| | CATCATCTGAAGATGGTACTTACACCATTCCACCAGATTCTACTGTTGATCAAACTACTGCT |
| | TTAACCCCATTCTGGGATACTCAATCTACTTTCTGGACCTCTTTTCAATCTGCTGGTGTTTCT |
| | CCATCTCAATTCGGTTATTCTTACCCAGAATTCAATGGTTTGAACTTGCAAGACCAAAAGGC |
| | TGTTAAGGATCATATTGCCGAAGTCGTCAATGAATTATACGGTCACAGAATGAGAAAGAC |
| | CTTTCCATTTCCACAATTGCAAGCTGTTTCTGTTGCTAAACAAGGTGATGCTGTTACTCCATC |
| | AGTTGCTACTGATTCTGTTTCTTCATCTACTACCCCAGCTGAAAATCCAGCTTCTAGAGAAG |
| | ATGCTTCTGATAAGGATACTGAACCTACATTGAACGTTGAAGTTGCTGCTCCAGGTGCTCA |
| | TTTGACTTCTACTAAGTACTGGGATTGGACCGCTAGAATTCACGTTAAGAAATATGAAGTC |
| | GGTGGTTCTTTCTCCGTCTTGTTGTTTTTGGGTGCTATTCCAGAAAATCCTGCAGATTGGAG |
| | AACATCTCCAAATTATGTCGGTGGTCATCATGCTTTCGTTAACTCTTCACCACAAAGATGTG |
| | CTAACTGTAGAGGTCAAGGTGATTTGGTTATTGAAGGTTTCGTCCATTTGAACGAAGCTAT |
| | TGCTAGACATGCACACTTGGATTCTTTTGACCCAACTGTTGTTAGACCTTACTTGACTAGAG |
| | AATTGCATTGGGGTGTTATGAAGGTTAACGGTACTGTTGTTCCATTGCAAGATGTTCCATC |
| | ATTGGAAGTTGTTGTCTTGTCTACTCCATTGACTTTACCACCAGGTGAACCATTTCCAGTTC |
| | CAGGTACTCCAGTTAACCATCATGATATTACACATGGTAGACCAGGTGGTTCTCATCATAC |
| | ACATTAA |
| SEQ ID NO: 6 | MSHFIVTGPVGGQTEGAPAPNRLEINDFVKNEEFFSLYVQALDIMYGLKQEELISFFQIGGIHGL |
| | PYVAWSDAGADDPAEPSGYCTHGSVLFPTVVHRPYVALYEQILHKYAGEIADKYTVDKPRWQK |
| | AAADLRQPFWDWAKNTLPPPEVISLDKVTITTPDGQRTQVDNPLRRYRFHPIDPSFPEPYSNW |
| | PATLRHPTSDGSDAKDNVKDLTTTLKADQPDITTKTYNLLTRVHTWPAFSNHTPGDGGSSSNS |
| | LEAIHDHIHDSVGGGGQMGDPSVAGFDPIFFLHHCQVDRLLALWSALNPGVWVNSSSSEDG |
| | TYTIPPDSTVDQTTALTPFWDTQSTFWTSFQSAGVSPSQFGYSYPEFNGLNLQDQKAVKDHIA |
| | EVVNELYGHRMRKTFPFPQLQAVSVAKQGDAVTPSVATDSVSSSTTPAENPASREDASDKDT |
| | EPTLNVEVAAPGAHLTSTKYWDWTARIHVKKYEVGGSFSVLLFLGAIPENPADWRTSPNYVG |
| | GHHAFVNSSPQRCANCRGQGDLVIEGFVHLNEAIARHAHLDSFDPTVVRPYLTRELHWGVM |
| | KVNGTVVPLQDVPSLEVVVLSTPLTLPPGEPFPVPGTPVNHHDITHGRPGGSHHTH |
| SEQ ID NO: 7 | ATGTCCCACTACTTGGTTACTGGTGCTACTGGTGGTTCTACTTCTGGTGCTGCTGCTCCAAA |
| | TAGATTGGAAATCAACGATTTCGTCAAGCAAGAAGATCAATTCTCCTTGTACATTCAAGCCT |
| | TGCAATATATCTACTCCTCCAAGTCCCAAGATGACATCGATTCTTTTTTCCAAATCGGTGGT |
| | ATTCACGGTTTGCCATATGTTCCATGGGATGGTGCTGGTAACAAACCAGTTGATACTGATG |
| | CTTGGGAAGGTTACTGTACTCATGGTTCTGTTTTGTTCCCAACTTTCCATAGACCATACGTC |
| | TTGTTGATTGAACAAGCTATTCAAGCTGCTGCTGTTGATATTGCTGCTACTTATATCGTTGA |
| | TAGAGCCAGATATCAAGATGCTGCCTTGAATTTGAGACAACCATATTGGGATTGGGCTAG |
| | AAATCCAGTTCCACCACCTGAAGTTATTTCTTTGGATGAAGTTACCATCGTCAACCCATCTG |
| | GTGAAAAGATTTCTGTTCCAAACCCATTGAGAAGATACACCTTCCATCCAATTGATCCATCT |
| | TTTCCAGAACCATACCAATCTTGGTCTACTACTTTAAGACACCCATTGTCTGATGATGCTAA |
| | CGCTTCTGATAATGTCCCAGAATTGAAAGCTACTTTGAGATCTGCTGGTCCACAATTGAAA |
| | ACTAAGACCTACAACTTGTTGACCAGAGTTCATACTTGGCCAGCTTTTTCTAATCATACTCC |
| | AGATGATGGTGGTTCCACCTCTAATTCTTTGGAAGGTATTCATGATTCCGTTCACGTTGATG |
| | TTGGTGGTAATGGTCAAATGTCTGATCCATCAGTTGCTGGTTTTGATCCAATCTTCTTTATG |
| | CATCATGCCCAAGTCGACAGATTATTGTCTTTGTGGTCTGCTTTGAATCCAAGAGTTTGGAT |
| | TACTGATGGTCCTTCTGGTGATGGTACTTGGACTATTCCACCAGATACTGTTGTTGGTAAA |
| | GATACTGATTTGACCCCATTCTGGAACACCCAATCTTCATATTGGATTTCTGCTAACGTTAC |
| | CGACACTTCTAAAATGGGTTATACCTACCCAGAATTCAACAACTTGGATATGGGTAACGAA |
| | GTTGCTGTTAGATCTGCTATTGCTGCACAAGTTAACAAGTTATATGGTGGTCCATTCACTAA |
| | GTTCGCTGCTGCTATACAACAACCATCTTCACAAACTACTGCTGATGCTTCTACTATTGGTA |
| | ATGTTACTTCCGATGCCTCCTCTCATTTGGTTGATTCTAAGATTAACCCAACCCCAAACAGA |
| | TCTATTGATGATGCACCTCAAGTTAAGATTGCCTCTACCTTGAGAAACAACGAACAAAAAG |
| | AATTTTGGGAATGGACCGCTAGAGTTCAAGTCAAAAAGTACGAAATTGGTGGTAGTTTCA |
| | AGGTCTTGTTCTTCTTGGGTTCAGTTCCATCTGATCCAAAAGAATGGGCTACTGATCCACAT |
| | TTTGTTGGTGCTTTTCATGGTTTCGTTAACTCCTCTGCTGAAAGATGTGCTAACTGTAGAAG |
| | ACAACAAGATGTTGTCTTGGAAGGTTTCGTCCATTTGAATGAAGGTATTGCCAACATCTCC |
| | AACTTGAATTCTTTCGATCCAATCGTTGTCGAACCATACTTGAAAGAAAACTTGCATTGGAG |
| | AGTTCAAAAGGTCAGTGGTGAAGTTGTTAATTTGGATGCTGCTACCTCATTGGAAGTTGTT |
| | GTTGTAGCTACCAGATTGGAATTGCCACCAGGTGAAATTTTTCCAGTTCCTGCTGAAACAC |
| | ATCATCATCACCATATTACACATGGTAGACCAGGTGGTTCAAGACATTCTGTTGCTTCATCT |
| | TCATCCTAA |
| SEQ ID NO 8: | MSHYLVTGATGGSTSGAAAPNRLEINDFVKQEDQFSLYIQALQYIYSSKSQDDIDSFFQIGGIFIG |
| | LPYVPWDGAGNKPVDTDAWEGYCTHGSVLFPTFHRPYVLLIEQAIQAAAVDIAATYIVDRARY |
| | QDAALNLRQPYWDWARNPVPPPEVISLDEVTIVNPSGEKISVPNPLRRYTFHPIDPSFPEPYQS |
| | WSTTLRHPLSDDANASDNVPELKATLRSAGPQLKTKTYNLLTRVHTWPAFSNHTPDDGGSTS |
| | NSLEGIHDSVHVDVGGNGQMSDPSVAGFDPIFFMHHAQVDRLLSLWSALNPRVWITDGPSG |
| | DGTVVTIPPDTVVGKDTDLTPFWNTQSSYWISANVTDTSKMGYTYPEFNNLDMGNEVAVRSA |
| | IAAQVNKLYGGPFTKFAAAIQQPSSQTTADASTIGNVTSDASSHLVDSKINPTPNRSIDDAPQV |
| | KIASTLRNNEQKEFWEWTARVQVKKYEIGGSFKVLFFLGSVPSDPKEWATDPHFVGAFHGFV |
| | NSSAERCANCRRQQDVVLEGFVHLNEGIANISNLNSFDPIVVEPYLKENLHWRVQKVSGEVVN |
| | LDAATSLEVVVVATRLELPPGEIFPVPAETHHHHHITHGRPGGSRHSVASSSS |
| SEQ ID NO: 9 | ATGTCCAGAGTTGTTATCACCGGTGTTTCTGGTACTATTGCTAACAGATTGGAAATCAACG |
| | ACTTCGTCAAGAACGACAAGTTCTTCTCATTGTACATTCAAGCCTTGCAAGTCATGTCATCT |
| | GTTCCACCACAAGAAAACGTTAGATCCTTCTTTCAAATCGGTGGTATTCATGGTTTGCCATA |
| | TACTCCATGGGATGGTATTACTGGTGATCAACCATTTGATCCAAATACTCAATGGGGTGGT |
| | TACTGTACTCATGGTTCTGTTTTGTTTCCAACTTGGCATAGACCATACGTCTTGTTGTATGAA |
| | CAAATCTTGCACAAGCACGTTCAAGATATTGCTGCTACTTATACCACTTCTGATAAGGCTGC |
| | TTGGGTTCAAGCTGCTGCTAATTTGAGACAACCATATTGGGATTGGGCTGCTAATGCTGTT |
| | CCTCCAGATCAAGTTATCGTTTCTAAGAAGGTTACCATCACTGGTTCTAACGGTCATAAGGT |
| | TGAAGTTGACAACCCATTATACCATTACAAGTTCCACCCAATCGATTCCTCATTTCCAAGAC |
| | CATATTCTGAATGGCCAACTACCTTAAGACAACCTAATTCTTCTAGACCAAACGCCACTGAT |
| | AATGTCGCTAAGTTGAGAAATGTTTTGAGAGCTTCCCAAGAAAACATCACCTCTAACACTT |
| | ACTCTATGTTGACCAGAGTTCATACTTGGAAGGCTTTCTCTAATCATACTGTTGGTGATGGT |
| | GGTTCTACCTCTAATTCTTTGGAAGCTATTCATGATGGTATCCACGTTGATGTAGGTGGTG |
| | GTGGTCATATGGGTGATCCAGCTGTTGCTGCTTTTGATCCTATTTTCTTCTTGCATCACTGCA |
| | ACGTCGACAGATTATTGTCTTTGTGGGCAGCTATTAACCCAGGTGTTTGGGTTTCTCCAGG |
| | TGATTCTGAAGATGGTACTTTCATTTTGCCACCTGAAGCTCCAGTTGATGTTTCTACTCCATT |
| | AACTCCATTCTCTAACACCGAAACTACTTTTTGGGCTTCTGGTGGTATTACAGATACAACTA |
| | AGTTGGGTTACACCTACCCAGAATTCAATGGTTTGGATTTGGGTAATGCTCAAGCTGTTAA |
| | GGCTGCAATTGGTAACATCGTTAACAGATTATACGGTGCCTCTGTTTTTTCTGGTTTTGCTG |
| | CTGCAACTTCTGCTATTGGTGCTGGTTCAGTTGCTTCTTTGGCTGCTGATGTTCCATTGGAA |
| | AAAGCTCCAGCTCCTGCTCCAGAAGCTGCCGCTCAACCACCAGTTCCAGCTCCAGCACATG |
| | TTGAACCAGCTGTTAGAGCTGTTTCTGTTCATGCTGCAGCTGCTCAACCTCATGCAGAACCA |
| | CCTGTTCATGTTTCTGCCGGTGGTCATCCATCTCCACATGGTTTTTATGATTGGACCGCTAG |
| | AATCGAATTCAAGAAGTACGAATTCGGTTCCTCCTTTTCCGTTTTGTTGTTTTTGGGTCCAG |
| | TTCCTGAAGATCCAGAACAATGGTTAGTTTCTCCAAATTTCGTTGGTGCTCATCATGCTTTT |
| | GTTAATTCTGCTGCTGGTCATTGTGCTAACTGTAGATCTCAAGGTAACGTTGTTGTTGAAG |
| | GTTTCGTTCATTTGACCAAGTACATTTCTGAACATGCCGGTTTGAGATCTTTGAACCCAGAA |
| | GTTGTTGAACCTTACTTGACCAACGAATTGCATTGGAGAGTTTTGAAAGCTGATGGTAGTG |
| | TTGGTCAATTGGAATCCTTGGAAGTTTCTGTTTATGGTACTCCAATGAACTTGCCAGTTGGT |
| | GCTATGTTTCCTGTTCCAGGTAATAGAAGACATTTCCATGGTATCACTCACGGTAGAGTTG |
| | GTGGTTCAAGACATGCTATAGTTTAA |
| SEQ ID NO: 10 | MSRVVITGVSGTIANRLEINDFVKNDKFFSLYIQALQVMSSVPPQENVRSFFQIGGIHGLPYTP |
| | WDGITGDQPFDPNTQWGGYCTHGSVLFPTVVHRPYVLLYEQILHKHVQDIAATYTTSDKAAW |
| | VQAAANLRQPYWDWAANAVPPDQVIVSKKVTITGSNGHKVEVDNPLYHYKFHPIDSSFPRPY |
| | SEWPTTLRQPNSSRPNATDNVAKLRNVLRASQENITSNTYSMLTRVHTWKAFSNHTVGDGG |
| | STSNSLEAIHDGIHVDVGGGGHMGDPAVAAFDPIFFLHHCNVDRLLSLWAAINPGVWVSPG |
| | DSEDGTFILPPEAPVDVSTPLTPFSNTETTFWASGGITDTTKLGYTYPEFNGLDLGNAQAVKAAI |
| | GNIVNRLYGASVFSGFAAATSAIGAGSVASLAADVPLEKAPAPAPEAAAQPPVPAPAHVEPAV |
| | RAVSVHAAAAQPHAEPPVHVSAGGHPSPHGFYDWTARIEFKKYEFGSSFSVLLFLGPVPEDPE |
| | QWLVSPNFVGAHHAFVNSAAGHCANCRSQGNVVVEGFVHLTKYISEHAGLRSLNPEVVEPYL |
| | TNELHWRVLKADGSVGQLESLEVSVYGTPMNLPVGAMFPVPGNRRHFHGITHGRVGGSRHA |
| | IV |
| SEQ ID NO: 11 | ATGGGGAAGCAAGAAGATGCAGAGCTCGTCATCATACCTTTCCCTTTCTCCGGACACATTC |
| | TCGCAACAATCGAACTCGCCAAACGTCTCATAAGTCAAGACAATCCTCGGATCCACACCAT |
| | CACCATCCTCTATTGGGGATTACCTTTTATTCCTCAAGCTGACACAATCGCTTTCCTCCGATC |
| | CCTAGTCAAAAATGAGCCTCGTATCCGTCTCGTTACGTTGCCCGAAGTCCAAGACCCTCCAC |
| | CAATGGAACTCTTTGTGGAATTTGCCGAATCTTACATTCTTGAATACGTCAAGAAAATGGTT |
| | CCCATCATCAGAGAAGCTCTCTCCACTCTCTTGTCTTCCCGCGATGAATCGGGTTCAGTTCG |
| | TGTGGCTGGATTGGTTCTTGACTTCTTCTGCGTCCCTATGATCGATGTAGGAAACGAGTTTA |
| | ATCTCCCTTCTTACATTTTCTTGACGTGTAGCGCAGGGTTCTTGGGTATGATGAAGTATCTT |
| | CCAGAGAGACACCGCGAAATCAAATCGGAATTCAACCGGAGCTTCAACGAGGAGTTGAAT |
| | CTCATTCCTGGTTATGTCAACTCTGTTCCTACTAAGGTTTTGCCGTCAGGTCTATTCATGAAA |
| | GAGACCTACGAGCCTTGGGTCGAACTAGCAGAGAGGTTTCCTGAAGCTAAGGGTATTTTG |
| | GTTAATTCATACACAGCTCTCGAGCCAAACGGTTTTAAATATTTCGATCGTTGTCCGGATAA |
| | CTACCCAACCATTTACCCAATCGGGCCGATATTATGCTCCAACGACCGTCCGAATTTGGACT |
| | CATCGGAACGAGATCGGATCATAACTTGGCTAGATGACCAACCCGAGTCATCGGTCGTGTT |
| | CCTCTGTTTCGGGAGCTTGAAGAATCTCAGCGCTACTCAGATCAACGAGATAGCTCAAGCC |
| | TTAGAGATCGTTGACTGCAAATTCATCTGGTCGTTTCGAACCAACCCGAAGGAGTACGCGA |
| | GCCCTTACGAGGCTCTACCACACGGGTTCATGGACCGGGTCATGGATCAAGGCATTGTTTG |
| | TGGTTGGGCTCCTCAAGTTGAAATCCTAGCCCATAAAGCTGTGGGAGGATTCGTATCTCAT |
| | TGTGGTTGGAACTCGATATTGGAGAGTTTGGGTTTCGGCGTTCCAATCGCCACGTGGCCG |
| | ATGTACGCGGAACAACAACTAAACGCGTTCACGATGGTGAAGGAGCTTGGTTTAGCCTTG |
| | GAGATGCGGTTGGATTACGTGTCGGAAGATGGAGATATAGTGAAAGCTGATGAGATCGC |
| | AGGAACCGTTAGATCTTTAATGGACGGTGTGGATGTGCCGAAGAGTAAAGTGAAGGAGA |
| | TTGCTGAGGCGGGAAAAGAAGCTGTGGACGGTGGATCTTCGTTTCTTGCGGTTAAAAGAT |
| | TCATCGGTGACTTGATCGACGGCGTTTCTATAAGTAAGTAG |
| SEQ ID NO: 12 | MGKQEDAELVIIPFPFSGHILATIELAKRLISQDNPRIHTITILYWGLPFIPQADTIAFLRSLVKNEP |
| | RIRLVTLPEVQDPPPMELFVEFAESYILEYVKKMVPIIREALSTLLSSRDESGSVRVAGLVLDFFCV |
| | PMIDVGNEFNLPSYIFLTCSAGFLGMMKYLPERHREIKSEFNRSFNEELNLIPGYVNSVPTKVLP |
| | SGLFMKETYEPWVELAERFPEAKGILVNSYTALEPNGFKYFDRCPDNYPTIYPIGPILCSNDRPNL |
| | DSSERDRIITWLDDQPESSVVFLCFGSLKNLSATQINEIAQALEIVDCKFIWSFRTNPKEYASPYE |
| | ALPHGFMDRVMDQGIVCGWAPQVEILAHKAVGGFVSHCGWNSILESLGFGVPIATVVPMYA |
| | EQQLNAFTMVKELGLALEMRLDYVSEDGDIVKADEIAGTVRSLMDGVDVPKSKVKEIAEAGKE |
| | AVDGGSSFLAVKRFIGDLIDGVSISK |
| SEQ ID NO: 13 | ATGGGGAAGCAAGAAGATGCAGAGCTCGTCATCATACCTTTCCCTTTCTCCGGACACATTC |
| | TCGCAACAATCGAACTCGCCAAACGTCTCATAAGTCAAGACAATCCTCGGATCCACACCAT |
| | CACCATCCTCTATTGGGGATTACCTTTTATTCCTCAAGCTGACACAATCGCTTTCCTCCGATC |
| | CCTAGTCAAAAATGAGCCTCGTATCCGTCTCGTTACGTTGCCCGAAGTCCAAGACCCTCCAC |
| | CAATGGAACTCTTTGTGGAATTTGCCGAATCTTACATTCTTGAATACGTCAAGAAAATGGTT |
| | CCCATCATCAGAGAAGCTCTCTCCACTCTCTTGTCTTCCCGCGATGAATCGGGTTCAGTTCG |
| | TGTGGCTGGATTGGTTCTTGACTTCTTCTGCGTCCCTATGATCGATGTAGGAAACGAGTTTA |
| | ATCTCCCTTCTTACATTTTCTTGACGTGTAGCGCAGGGTTCTTGGGTATGATGAAGTATCTT |
| | CCAGAGAGACACCGCGAAATCAAATCGGAATTCAACCGGAGCTTCAACGAGGAGTTGAAT |
| | CTCATTCCCGGGTTTGTTAACTCCGTTCCGGTTAAAGTTTTGCCACCGGGTTTGTTCACGAC |
| | TGAGTCTTACGAAGCTTGGGTCGAAATGGCGGAAAGGTTCCCTGAAGCCAAGGGTATTTT |
| | GGTCAATTCATTTGAATCTCTAGAACGTAACGCTTTTGATTATTTCGATCGTCGTCCGGATA |
| | ATTACCCACCCGTTTACCCAATCGGGCCAATTCTATGCTCCAACGATCGTCCGAATTTGGAT |
| | TTATCGGAACGAGACCGGATCTTGAAATGGCTCGATGACCAACCCGAGTCATCTGTTGTGT |
| | TTCTCTGCTTCGGGAGCTTGAAGAGTCTCGCTGCGTCTCAGATTAAAGAGATCGCTCAAGC |
| | CTTAGAGCTCGTCGGAATCAGATTCCTCTGGTCGATTCGAACGGACCCGAAGGAGTACGC |
| | GAGCCCGAACGAGATTTTACCGGACGGGTTTATGAACCGAGTCATGGGTTTGGGCCTTGT |
| | TTGTGGTTGGGCTCCTCAAGTTGAAATTCTGGCCCATAAAGCAATTGGAGGGTTCGTGTCA |
| | CACTGCGGTTGGAACTCGATATTGGAGAGTTTGCGTTTCGGAGTTCCAATTGCCACGTGGC |
| | CAATGTACGCGGAACAACAACTAAACGCGTTCACGATTGTGAAGGAGCTTGGTTTGGCGT |
| | TGGAGATGCGGTTGGATTACGTGTCGGAATATGGAGAAATCGTGAAAGCTGATGAAATCG |
| | CAGGAGCCGTACGATCTTTGATGGACGGTGAGGATGTGCCGAGGAGGAAACTGAAGGAG |
| | ATTGCGGAGGCGGGAAAAGAGGCTGTGATGGACGGTGGATCTTCGTTTGTTGCGGTTAA |
| | AAGATTCATAGATGGGCTTTGA |
| SEQ ID NO: 14 | MGKQEDAELVIIPFPFSGHILATIELAKRLISQDNPRIHTITILYWGLPFIPQADTIAFLRSLVKNEP |
| | RIRLVTLPEVQDPPPMELFVEFAESYILEYVKKMVPIIREALSTLLSSRDESGSVRVAGLVLDFFCV |
| | PMIDVGNEFNLPSYIFLTCSAGFLGMMKYLPERHREIKSEFNRSFNEELNLIPGFVNSVPVKVLP |
| | PGLFTTESYEAWVEMAERFPEAKGILVNSFESLERNAFDYFDRRPDNYPPVYPIGPILCSNDRPN |
| | LDLSERDRILKWLDDQPESSVVFLCFGSLKSLAASQIKEIAQALELVGIRFLWSIRTDPKEYASPNE |
| | ILPDGFMNRVMGLGLVCGWAPQVEILAHKAIGGFVSHCGWNSILESLRFGVPIATWPMYAE |
| | QQLNAFTIVKELGLALEMRLDYVSEYGEIVKADEIAGAVRSLMDGEDVPRRKLKEIAEAGKEAV |
| | MDGGSSFVAVKRFIDGL |
| SEQ ID NO: 15 | ATGGGGAAGCAAGAAGATGCAGAGCTCGTCATCATACCTTTCCCTTTCTCCGGACACATTC |
| | TCGCAACAATCGAACTCGCCAAACGTCTCATAAGTCAAGACAATCCTCGGATCCACACCAT |
| | CACCATCCTCTATTGGGGATTACCTTTTATTCCTCAAGCTGACACAATCGCTTTCCTCCGATC |
| | CCTAGTCAAAAATGAGCCTCGTATCCGTCTCGTTACGTTGCCCGAAGTCCAAGACCCTCCAC |
| | CAATGGAACTCTTTGTGGAATTTGCCGAATCTTACATTCTTGAATACGTCAAGAAAATGGTT |
| | CCCATCATCAGAGAAGCTCTCTCCACTCTCTTGTCTTCCCGCGATGAATCGGGTTCAGTTCG |
| | TGTGGCTGGATTGGTTCTTGACTTCTTCTGCGTCCCTATGATCGATGTAGGAAACGAGTTTA |
| | ATCTCCCTTCTTACATTTTCTTGACGTGTAGCGCAGGGTTCTTGGGTATGATGAAGTATCTT |
| | CCAGAGAGACACCGCGAAATCAAATCGGAATTCAACCGGAGCTTCAACGAGGAGTTGAAT |
| | CTCATTCCTGGTTATGTCAACTCTGTTCCTACTAAGGTTTTGCCGTCAGGTCTATTCATGAAA |
| | GAGACCTACGAGCCTTGGGTCGAACTAGCAGAGAGGTTTCCTGAAGCTAAGGGTATTTTG |
| | GTTAATTCATACACAGCTCTCGAGCCAAACGGTTTTAAATATTTCGATCGTTGTCCGGATAA |
| | CTACCCAACCATTTACCCAATCGGGCCCATTCTATGCTCCAACGATCGTCCGAATTTGGATT |
| | TATCGGAACGAGACCGGATCTTGAAATGGCTCGATGACCAACCCGAGTCATCTGTTGTGTT |
| | TCTCTGCTTCGGGAGCTTGAAGAGTCTCGCTGCGTCTCAGATTAAAGAGATCGCTCAAGCC |
| | TTAGAGCTCGTCGGAATCAGATTCCTCTGGTCGATTCGAACGGACCCGAAGGAGTACGCG |
| | AGCCCGAACGAGATTTTACCGGACGGGTTTATGAACCGAGTCATGGGTTTGGGCCTTGTTT |
| | GTGGTTGGGCTCCTCAAGTTGAAATTCTGGCCCATAAAGCAATTGGAGGGTTCGTGTCACA |
| | CTGCGGTTGGAACTCGATATTGGAGAGTTTGCGTTTCGGAGTTCCAATTGCCACGTGGCCA |
| | ATGTACGCGGAACAACAACTAAACGCGTTCACGATTGTGAAGGAGCTTGGTTTGGCGTTG |
| | GAGATGCGGTTGGATTACGTGTCGGAATATGGAGAAATCGTGAAAGCTGATGAAATCGCA |
| | GGAGCCGTACGATCTTTGATGGACGGTGAGGATGTGCCGAGGAGGAAACTGAAGGAGAT |
| | TGCGGAGGCGGGAAAAGAGGCTGTGATGGACGGTGGATCTTCGTTTGTTGCGGTTAAAA |
| | GATTCATAGATGGGCTTTGA |
| SEQ ID NO: 16 | MGKQEDAELVIIPFPFSGHILATIELAKRLISQDNPRIHTITILYWGLPFIPQADTIAFLRSLVKNEP |
| | RIRLVTLPEVQDPPPMELFVEFAESYILEYVKKMVPIIREALSTLLSSRDESGSVRVAGLVLDFFCV |
| | PMIDVGNEFNLPSYIFLTCSAGFLGMMKYLPERHREIKSEFNRSFNEELNLIPGYVNSVPTKVLP |
| | SGLFMKETYEPWVELAERFPEAKGILVNSYTALEPNGFKYFDRCPDNYPTIYPIGPILCSNDRPNL |
| | DLSERDRILKWLDDQPESSVVFLCFGSLKSLAASQIKEIAQALELVGIRFLWSIRTDPKEYASPNEI |
| | LPDGFMNRVMGLGLVCGWAPQVEILAHKAIGGFVSHCGWNSILESLRFGVPIATWPMYAEQ |
| | QLNAFTIVKELGLALEMRLDYVSEYGEIVKADEIAGAVRSLMDGEDVPRRKLKEIAEAGKEAVM |
| | DGGSSFVAVKRFIDGL |
| SEQ ID NO: 17 | ATGGGGAAGCAAGAAGATGCAGAGCTCGTCATCATACCTTTCCCTTTCTCCGGACACATTC |
| | TCGCAACAATCGAACTCGCCAAACGTCTCATAAGTCAAGACAATCCTCGGATCCACACCAT |
| | CACCATCCTCTATTGGGGATTACCTTTTATTCCTCAAGCTGACACAATCGCTTTCCTCCGATC |
| | CCTAGTCAAAAATGAGCCTCGTATCCGTCTCGTTACGTTGCCCGAAGTCCAAGACCCTCCAC |
| | CAATGGAACTCTTTGTGGAATTTGCCGAATCTTACATTCTTGAATACGTCAAGAAAATGGTT |
| | CCCATCATCAGAGAAGCTCTCTCCACTCTCTTGTCTTCCCGCGATGAATCGGGTTCAGTTCG |
| | TGTGGCTGGATTGGTTCTTGACTTCTTCTGCGTCCCTATGATCGATGTAGGAAACGAGTTTA |
| | ATCTCCCTTCTTACATTTTCTTGACGTGTAGCGCAGGGTTCTTGGGTATGATGAAGTATCTT |
| | CCAGAGAGACACCGCGAAATCAAATCGGAATTCAACCGGAGCTTCAACGAGGAGTTGAAT |
| | CTCATTCCTGGTTATGTCAACTCTGTTCCTACTAAGGTTTTGCCGTCAGGTCTATTCATGAAA |
| | GAGACCTACGAGCCTTGGGTCGAACTAGCAGAGAGGTTTCCTGAAGCTAAGGGTATTTTG |
| | GTTAATTCATACACAGCTCTCGAGCCAAACGGTTTTAAATATTTCGATCGTTGTCCGGATAA |
| | CTACCCAACCATTTACCCAATCGGGCCCATTTTGAACCTTGAAAACAAAAAAGACGATGCT |
| | AAAACCGACGAGATTATGAGGTGGTTAAATGAGCAACCGGAAAGCTCGGTTGTGTTTTTA |
| | TGTTTCGGAAGCATGGGTAGCTTTAACGAGAAACAAGTGAAGGAGATTGCGGTTGCGATT |
| | GAAAGAAGTGGACATAGATTTTTATGGTCGCTTCGTCGTCCGACACCGAAAGAAAAGATA |
| | GAGTTTCCGAAAGAATATGAAAACTTGGAAGAAGTTCTTCCAGAGGGATTCCTTAAACGTA |
| | CATCAAGCATCGGGAAGGTGATCGGGTGGGCCCCACAAATGGCGGTGTTGTCTCACCCGT |
| | CAGTTGGTGGGTTTGTGTCGCATTGTGGTTGGAACTCGACATTGGAGAGTATGTGGTGTG |
| | GGGTTCCGATGGCAGCTTGGCCATTATATGCTGAACAAACGTTGAATGCTTTTCTACTTGT |
| | GGTGGAACTGGGATTGGCGGCGGAGATTAGGATGGATTATCGGACGGATACGAAAGCGG |
| | GGTATGACGGTGGGATGGAGGTGACGGTGGAGGAGATTGAAGATGGAATTAGGAAGTT |
| | GATGAGTGATGGTGAGATTAGAAATAAGGTGAAAGATGTGAAAGAGAAGAGTAGAGCTG |
| | CGGTTGTTGAAGGTGGATCTTCTTACGCATCCATTGGAAAATTCATCGAGCATGTATCGAA |
| | TGTTACGATTTAA |
| SEQ ID NO: 18 | MGKQEDAELVIIPFPFSGHILATIELAKRLISQDNPRIHTITILYWGLPFIPQADTIAFLRSLVKNEP |
| | RIRLVTLPEVQDPPPMELFVEFAESYILEYVKKMVPIIREALSTLLSSRDESGSVRVAGLVLDFFCV |
| | PMIDVGNEFNLPSYIFLTCSAGFLGMMKYLPERHREIKSEFNRSFNEELNLIPGYVNSVPTKVLP |
| | SGLFMKETYEPWVELAERFPEAKGILVNSYTALEPNGFKYFDRCPDNYPTIYPIGPILNLENKKD |
| | DAKTDEIMRWLNEQPESSVVFLCFGSMGSFNEKQVKEIAVAIERSGHRFLWSLRRPTPKEKIEF |
| | PKEYENLEEVLPEGFLKRTSSIGKVIGWAPQMAVLSHPSVGGFVSHCGWNSTLESMWCGVP |
| | MAAWPLYAEQTLNAFLLVVELGLAAEIRMDYRTDTKAGYDGGMEVTVEEIEDGIRKLMSDGE |
| | IRNKVKDVKEKSRAAVVEGGSSYASIGKFIEHVSNVTI |
| SEQ ID NO: 19 | ATGGCGAAGCAGCAAGAAGCAGAGCTCATCTTCATCCCATTTCCAATCCCCGGACACATTC |
| | TCGCCACAATCGAACTCGCGAAACGTCTCATCAGTCACCAACCTAGTCGGATCCACACCAT |
| | CACCATCCTCCATTGGAGCTTACCTTTTCTTCCTCAATCTGACACTATCGCCTTCCTCAAATC |
| | CCTAATCGAAACAGAGTCTCGTATCCGTCTCATTACCTTACCCGATGTCCAAAACCCTCCAC |
| | CAATGGAGCTATTTGTGAAAGCTTCCGAATCTTACATTCTTGAATACGTCAAGAAAATGGT |
| | TCCTTTGGTCAGAAACGCTCTCTCCACTCTCTTGTCTTCTCGTGATGAATCGGATTCAGTTCA |
| | TGTCGCCGGATTAGTTCTTGATTTCTTCTGTGTCCCTTTGATCGATGTCGGAAACGAGTTTA |
| | ATCTCCCTTCTTACATCTTCTTGACGTGTAGCGCAAGTTTCTTGGGTATGATGAAGTATCTTC |
| | TGGAGAGAAACCGCGAAACCAAACCGGAACTTAACCGGAGCTCTGACGAGGAAACAATA |
| | TCAGTTCCTGGTTTTGTTAACTCCGTTCCGGTTAAAGTTTTGCCACCGGGTTTGTTCACGAC |
| | TGAGTCTTACGAAGCTTGGGTCGAAATGGCGGAAAGGTTCCCTGAAGCCAAGGGTATTTT |
| | GGTCAATTCATTTGAATCTCTAGAACGTAACGCTTTTGATTATTTCGATCGTCGTCCGGATA |
| | ATTACCCACCCGTTTACCCAATCGGGCCCATTTTGAACCTTGAAAACAAAAAAGACGATGC |
| | TAAAACCGACGAGATTATGAGGTGGTTAAATGAGCAACCGGAAAGCTCGGTTGTGTTTTT |
| | ATGTTTCGGAAGCATGGGTAGCTTTAACGAGAAACAAGTGAAGGAGATTGCGGTTGCGAT |
| | TGAAAGAAGTGGACATAGATTTTTATGGTCGCTTCGTCGTCCGACACCGAAAGAAAAGAT |
| | AGAGTTTCCGAAAGAATATGAAAACTTGGAAGAAGTTCTTCCAGAGGGATTCCTTAAACGT |
| | ACATCAAGCATCGGGAAGGTGATCGGGTGGGCCCCACAAATGGCGGTGTTGTCTCACCCG |
| | TCAGTTGGTGGGTTTGTGTCGCATTGTGGTTGGAACTCGACATTGGAGAGTATGTGGTGT |
| | GGGGTTCCGATGGCAGCTTGGCCATTATATGCTGAACAAACGTTGAATGCTTTTCTACTTG |
| | TGGTGGAACTGGGATTGGCGGCGGAGATTAGGATGGATTATCGGACGGATACGAAAGCG |
| | GGGTATGACGGTGGGATGGAGGTGACGGTGGAGGAGATTGAAGATGGAATTAGGAAGT |
| | TGATGAGTGATGGTGAGATTAGAAATAAGGTGAAAGATGTGAAAGAGAAGAGTAGAGCT |
| | GCGGTTGTTGAAGGTGGATCTTCTTACGCATCCATTGGAAAATTCATCGAGCATGTATCGA |
| | ATGTTACGATTTAA |
| SEQ ID NO: 20 | MAKQQEAELIFIPFPIPGHILATIELAKRLISHQPSRIHTITILHWSLPFLPQSDTIAFLKSLIETESRIR |
| | LITLPDVQNPPPMELFVKASESYILEYVKKMVPLVRNALSTLLSSRDESDSVHVAGLVLDFFCVPL |
| | IDVGNEFNLPSYIFLTCSASFLGMMKYLLERNRETKPELNRSSDEETISVPGFVNSVPVKVLPPGL |
| | FTTESYEAWVEMAERFPEAKGILVNSFESLERNAFDYFDRRPDNYPPVYPIGPILNLENKKDDA |
| | KTDEIMRWLNEQPESSVVFLCFGSMGSFNEKQVKEIAVAIERSGHRFLWSLRRPTPKEKIEFPK |
| | EYENLEEVLPEGFLKRTSSIGKVIGWAPQMAVLSHPSVGGFVSHCGWNSTLESMWCGVPMA |
| | AWPLYAEQTLNAFLLVVELGLAAEIRMDYRTDTKAGYDGGMEVTVEEIEDGIRKLMSDGElRN |
| | KVKDVKEKSRAAVVEGGSSYASIGKFIEHVSNVTI |
| SEQ ID NO: 21 | ATGAAGACAGCAGAGCTCATATTCGTTCCTCTGCCGGAGACCGGCCATCTCTTGTCAACGA |
| | TCGAGTTTGGAAAGCGTCTACTCAATCTAGACCGTCGGATTTCTATGATTACAATCCTCTCC |
| | ATGAATCTTCCTTACGCTCCTCACGCCGACGCTTCTCTTGCTTCGCTAACAGCCTCCGAGCC |
| | TGGTATCCGAATCATCAGTCTCCCGGAGATCCACGATCCACCTCCGATCAAGCTTCTTGACA |
| | CTTCCTCCGAGACTTACATCCTCGATTTCATCCATAAAAACATACCTTGTCTCAGAAAAACC |
| | ATCCAAGATTTAGTCTCATCATCATCATCTTCCGGAGGTGGTAGTAGTCATGTCGCCGGCTT |
| | GATTCTTGATTTCTTCTGCGTTGGTTTGATCGACATCGGCCGTGAGGTAAACCTTCCTTCCT |
| | ATATCTTCATGACTTCCAACTTTGGTTTCTTAGGGGTTCTACAGTATCTCCCGGAACGACAA |
| | CGTTTGACTCCGTCGGAGTTCGATGAGAGCTCCGGCGAGGAAGAGTTACATATTCCGGCG |
| | TTTGTGAACCGTGTTCCCGCCAAGGTTCTGCCGCCAGGTGTGTTCGATAAACTCTCTTACG |
| | GGTCTCTGGTCAAAATCGGCGAGCGATTACATGAAGCCAAGGGTATTTTGGTTAATTCATT |
| | TACCCAAGTGGAGCCTTATGCTGCTGAACATTTTTCTCAAGGACGAGATTACCCTCACGTG |
| | TATCCTGTTGGGCCGGTTCTCAACTTAACGGGCCGTACAAATCCGGGTCTAGCTTCGGCCC |
| | AATATAAAGAGATGATGAAGTGGCTTGACGAGCAACCAGACTCGTCGGTTTTGTTCCTGTG |
| | TTTCGGGAGCATGGGAGTCTTCCCTGCACCTCAGATCACAGAGATTGCTCACGCGCTCGAG |
| | CTTATCGGGTGCAGGTTCATCTGGGCGATCCGTACGAACATGGCGGGAGATGGCGATCCT |
| | CAGGAGCCGCTTCCAGAAGGATTTGTCGATCGAACAATGGGCCGTGGAATTGTGTGTAGT |
| | TGGGCTCCACAAGTGGATATCTTGGCCCACAAGGCAACAGGTGGATTCGTTTCTCACTGCG |
| | GGTGGAATTCCGTCCAAGAGAGTCTATGGTACGGTGTACCTATTGCAACGTGGCCAATGT |
| | ATGCGGAGCAACAACTGAACGCATTTGAGATGGTGAAGGAGTTGGGCTTAGCAGTGGAG |
| | ATAAGGCTTGACTACGTGGCGGATGGTGATAGGGTTACTTTGGAGATCGTGTCAGCCGAT |
| | GAAATAGCCACAGCCGTCCGATCATTGATGGATAGTGATAACCCCGTGAGAAAGAAGGTT |
| | ATAGAAAAATCTTCAGTGGCGAGGAAAGCTGTTGGTGATGGTGGGTCTTCTACGGTGGCC |
| | ACATGTAATTTTATCAAAGATATTCTTGGGGATCACTTTTGA |
| SEQ ID NO: 22 | MKTAELIFVPLPETGHLLSTIEFGKRLLNLDRRISMITILSMNLPYAPHADASLASLTASEPGIRIISL |
| | PEIHDPPPIKLLDTSSETYILDFIHKNIPCLRKTIQDLVSSSSSSGGGSSHVAGLILDFFCVGLIDIGR |
| | EVNLPSYIFMTSNFGFLGVLQYLPERQRLTPSEFDESSGEEELHIPAFVNRVPAKVLPPGVFDKLS |
| | YGSLVKIGERLHEAKGILVNSFTQVEPYAAEHFSQGRDYPHVYPVGPVLNLTGRTNPGLASAQY |
| | KEMMKWLDEQPDSSVLFLCFGSMGVFPAPQITEIAHALELIGCRFIWAIRTNMAGDGDPQEP |
| | LPEGFVDRTMGRGIVCSWAPQVDILAHKATGGFVSHCGWNSVQESLWYGVPIATWPMYAE |
| | QQLNAFEMVKELGLAVEIRLDYVADGDRVTLEIVSADEIATAVRSLMDSDNPVRKKVIEKSSVA |
| | RKAVGDGGSSTVATCNFIKDILGDHF |
| SEQ ID NO: 23 | ATGTCCACCTCAGAGCTTGTTTTCATCCCATCTCCCGGAGCTGGCCATCTACCACCAACGGT |
| | CGAGCTCGCAAAGCTTCTGTTACATCGCGATCAACGACTTTCGGTCACAATCATCGTCATG |
| | AATCTCTGGTTAGGTCCAAAACACAACACTGAAGCACGACCTTGTGTTCCCAGTTTACGGT |
| | TCGTTGACATCCCTTGCGATGAGTCCACCATGGCTCTCATCTCACCCAATACTTTTATATCTG |
| | CGTTCGTTGAACACCACAAACCGCGTGTTAGAGACATAGTCCGAGGTATAATTGAGTCTGA |
| | CTCGGTTCGACTCGCTGGGTTCGTTCTTGATATGTTTTGTATGCCGATGAGTGATGTTGCAA |
| | ACGAGTTTGGAGTTCCGAGTTACAATTATTTCACATCCGGTGCAGCCACGTTAGGGTTGAT |
| | GTTTCACCTTCAATGGAAACGTGATCATGAAGGTTATGATGCAACCGAGTTGAAAAACTCG |
| | GATACTGAGTTGTCTGTTCCGAGTTATGTTAACCCGGTTCCTGCTAAGGTTTTACCGGAAGT |
| | GGTGTTGGATAAAGAAGGTGGGTCCAAAATGTTTCTTGACCTTGCGGAAAGGATTCGCGA |
| | GTCGAAGGGTATAATAGTAAATTCATGTCAGGCGATTGAAAGACACGCGCTCGAGTACCT |
| | TTCAAGCAACAATAACGGTATCCCACCTGTTTTCCCGGTTGGTCCGATTTTGAACCTTGAAA |
| | ACAAAAAAGACGATGCTAAAACCGACGAGATTATGAGGTGGTTAAATGAGCAACCGGAA |
| | AGCTCGGTTGTGTTTTTATGTTTCGGAAGCATGGGTAGCTTTAACGAGAAACAAGTGAAG |
| | GAGATTGCGGTTGCGATTGAAAGAAGTGGACATAGATTTTTATGGTCGCTTCGTCGTCCGA |
| | CACCGAAAGAAAAGATAGAGTTTCCGAAAGAATATGAAAACTTGGAAGAAGTTCTTCCAG |
| | AGGGATTCCTTAAACGTACATCAAGCATCGGGAAGGTGATCGGGTGGGCCCCACAAATGG |
| | CGGTGTTGTCTCACCCGTCAGTTGGTGGGTTTGTGTCGCATTGTGGTTGGAACTCGACATT |
| | GGAGAGTATGTGGTGTGGGGTTCCGATGGCAGCTTGGCCATTATATGCTGAACAAACGTT |
| | GAATGCTTTTCTACTTGTGGTGGAACTGGGATTGGCGGCGGAGATTAGGATGGATTATCG |
| | GACGGATACGAAAGCGGGGTATGACGGTGGGATGGAGGTGACGGTGGAGGAGATTGAA |
| | GATGGAATTAGGAAGTTGATGAGTGATGGTGAGATTAGAAATAAGGTGAAAGATGTGAA |
| | AGAGAAGAGTAGAGCTGCGGTTGTTGAAGGTGGATCTTCTTACGCATCCATTGGAAAATT |
| | CATCGAGCATGTATCGAATGTTACGATTTAA |
| SEQ ID NO: 24 | MSTSELVFIPSPGAGHLPPTVELAKLLLHRDQRLSVTIIVMNLWLGPKHNTEARPCVPSLRFVDI |
| | PCDESTMALISPNTFISAFVEHHKPRVRDIVRGIIESDSVRLAGFVLDMFCMPMSDVANEFGVP |
| | SYNYFTSGAATLGLMFHLQWKRDHEGYDATELKNSDTELSVPSYVNPVPAKVLPEVVLDKEGG |
| | SKMFLDLAERIRESKGIIVNSCQAIERHALEYLSSNNNGIPPVFPVGPILNLENKKDDAKTDEIMR |
| | WLNEQPESSVVFLCFGSMGSFNEKQVKEIAVAIERSGHRFLWSLRRPTPKEKIEFPKEYENLEEV |
| | LPEGFLKRTSSIGKVIGWAPQMAVLSHPSVGGFVSHCGWNSTLESMWCGVPMAAWPLYAE |
| | QTLNAFLLVVELGLAAEIRMDYRTDTKAGYDGGMEVTVEEIEDGIRKLMSDGEIRNKVKDVKE |
| | KSRAAVVEGGSSYASIGKFIEHVSNVTI |
| SEQ ID NO: 25 | ATGGAGGAATCCAAAACACCTCACGTTGCGATCATACCAAGTCCGGGAATGGGTCATCTC |
| | ATACCACTCGTCGAGTTTGCTAAACGACTCGTCCATCTTCACGGCCTCACCGTTACCTTCGT |
| | CATCGCCGGCGAAGGTCCACCATCAAAAGCTCAGAGAACCGTCCTCGACTCTCTCCCTTCTT |
| | CAATCTCCTCCGTCTTTCTCCCTCCTGTTGATCTCACCGATCTCTCTTCGTCCACTCGCATCGA |
| | ATCTCGGATCTCCCTCACCGTGACTCGTTCAAACCCGGAGCTCCGGAAAGTCTTCGACTCG |
| | TTCGTGGAGGGAGGTCGTTTGCCAACGGCGCTCGTCGTCGATCTCTTCGGTACGGACGCTT |
| | TCGACGTGGCCGTAGAATTTCACGTGCCACCGTATATTTTCTACCCAACAACGGCCAACGT |
| | CTTGTCGTTTTTTCTCCATTTGCCTAAACTAGACGAAACGGTGTCGTGTGAGTTCAGGGAAT |
| | TAACCGAACCGCTTATGCTTCCTGGATGTGTACCGGTTGCCGGGAAAGATTTCCTTGACCC |
| | GGCCCAAGACCGGAAAGACGATGCATACAAATGGCTTCTCCATAACACCAAGAGGTACAA |
| | AGAAGCCGAAGGTATTCTTGTGAATACCTTCTTTGAGCTAGAGCCAAATGCTATAAAGGCC |
| | TTGCAAGAACCGGGTCTTGATAAACCACCGGTTTATCCGGTTGGACCGTTGGTTAACATTG |
| | GTAAGCAAGAGGCTAAGCAAACCGAAGAGTCTGAATGTTTAAAGTGGTTGGATAACCAGC |
| | CGCTCGGTTCGGTTTTATATGTGTCCTTTGGTAGTGGCGGTACCCTCACATGTGAGCAGCT |
| | CAATGAGCTTGCTCTTGGTCTTGCAGATAGTGAGCAACGGTTTCTTTGGGTCATACGAAGT |
| | CCTAGTGGGATCGCTAATTCGTCGTATTTTGATTCACATAGCCAAACAGATCCATTGACATT |
| | TTTACCACCGGGATTTTTAGAGCGGACTAAAAAAAGAGGTTTTGTGATCCCTTTTTGGGCT |
| | CCACAAGCCCAAGTCTTGGCGCATCCATCCACGGGAGGATTTTTAACTCATTGTGGATGGA |
| | ATTCGACTCTAGAGAGTGTAGTAAGCGGTATTCCACTTATAGCATGGCCATTATACGCAGA |
| | ACAGAAGATGAATGCGGTTTTGTTGAGTGAAGATATTCGTGCGGCACTTAGGCCGCGTGC |
| | CGGGGACGATGGGTTAGTTAGAAGAGAAGAGGTGGCTAGAGTGGTAAAAGGATTGATG |
| | GAAGGTGAAGAAGGCAAAGGAGTGAGGAACAAGATGAAGGAGTTGAAGGAAGCAGCTT |
| | GTAGGGTGTTGAAGGATGATGGGACTTCGACAAAAGCACTTAGTCTTGTGGCCTTAAAGT |
| | GGAAAGCCCACAAAAAAGAGTTAGAGCAAAATGGCAACCACTAA |
| SEQ ID NO: 26 | MEESKTPHVAIIPSPGMGHLIPLVEFAKRLVHLHGLTVTFVIAGEGPPSKAQRTVLDSLPSSISSV |
| | FLPPVDLTDLSSSTRIESRISLTVTRSNPELRKVFDSFVEGGRLPTALVVDLFGTDAFDVAVEFHV |
| | PPYIFYPTTANVLSFFLHLPKLDETVSCEFRELTEPLMLPGCVPVAGKDFLDPAQDRKDDAYKW |
| | LLHNTKRYKEAEGILVNTFFELEPNAIKALQEPGLDKPPVYPVGPLVNIGKQEAKQTEESECLKW |
| | LDNQPLGSVLYVSFGSGGTLTCEQLNELALGLADSEQRFLWVIRSPSGIANSSYFDSHSQTDPLT |
| | FLPPGFLERTKKRGFVIPFWAPQAQVLAHPSTGGFLTHCGWNSTLESVVSGIPLIAWPLYAEQK |
| | MNAVLLSEDIRAALRPRAGDDGLVRREEVARVVKGLMEGEEGKGVRNKMKELKEAACRVLK |
| | DDGTSTKALSLVALKWKAHKKELEQNGNH |
| SEQ ID NO: 27 | ATGGCGGAAGCAAACACTCCACACATAGCAATCATGCCGAGTCCCGGTATGGGTCACCTT |
| | ATCCCATTCGTCGAGTTAGCAAAGCGACTCGTTCAGCACGACTGTTTCACCGTCACAATGA |
| | TCATCTCCGGTGAAACTTCGCCGTCTAAGGCACAAAGATCCGTTCTCAACTCTCTCCCTTCC |
| | TCCATAGCCTCCGTATTTCTCCCTCCCGCCGATCTTTCCGATGTTCCCTCCACAGCGCGAATC |
| | GAAACTCGGGCCATGCTCACCATGACTCGTTCCAATCCGGCGCTCCGGGAGCTTTTTGGCT |
| | CTTTATCAACGAAGAAAAGTCTCCCGGCGGTTCTCGTCGTCGATATGTTTGGTGCGGATGC |
| | GTTCGACGTGGCCGTTGACTTCCACGGGTCACCATACATTTTCTATGCATCCAATGCAAAC |
| | GTCTTGTCGTTTTTTCTTCACTTGCCGAAACTAGACAAAACGGTGTCGTGTGAGTTTAGGTA |
| | CTTAACCGAACCGCTTAAGATTCCCGGCTGTGTCCCGATAACCGGTAAGGACTTTCTTGAT |
| | ACGGTTCAAGACCGAAACGACGACGCATACAAATTGCTTCTCCATAACACCAAGAGGTAC |
| | AAAGAAGCTAAAGGGATTCTAGTGAATTCCTTCGTTGATTTAGAGTCGAATGCAATAAAG |
| | GCCTTACAAGAACCGGCTCCTGATAAACCAACGGTATACCCGATTGGGCCGCTGGTTAACA |
| | CAAGTTCATCTAATGTTAACTTGGAAGACAAGTTCGGATGTTTAAGTTGGCTAGACAACCA |
| | ACCATTCGGCTCGGTTCTATACATATCATTTGGAAGCGGCGGAACACTTACATGTGAGCAG |
| | TTTAATGAGCTTGCTATTGGTCTTGCGGAGAGCGGAAAACGGTTTATTTGGGTCATACGAA |
| | GTCCAAGCGAGATAGTTAGTTCGTCGTATTTCAATCCACACAGCGAGACAGACCCCTTTTC |
| | GTTTTTACCAATTGGGTTCTTAGACCGAACCAAAGAGAAAGGTTTGGTGGTTCCATCATGG |
| | GCTCCACAGGTTCAAATCCTGGCTCATCCATCCACATGCGGGTTTTTAACACACTGTGGAT |
| | GGAATTCGACCTTAGAAAGCATTGTAAACGGTGTACCACTCATAGCGTGGCCTTTATTCGC |
| | GGAGCAAAAGATGAATACATTGCTACTCGTGGAGGATGTTGGAGCGGCTCTAAGAATCCA |
| | TGCGGGTGAAGATGGGATTGTACGGAGGGAAGAAGTGGTGAGAGTGGTGAAGGCACTG |
| | ATGGAAGGTGAAGAGGGAAAAGCCATAGGAAATAAAGTGAAGGAGTTGAAAGAAGGAG |
| | TTGTTAGAGTCTTGGGTGACGATGGATTGTCCAGCAAGTCATTTGGTGAAGTTTTGTTAAA |
| | GTGGAAAACGCACCAGCGAGATATCAACCAAGAGACGTCCCACTAG |
| SEQ ID NO: 28 | MAEANTPHIAIMPSPGMGHLIPFVELAKRLVQHDCFTVTMIISGETSPSKAQRSVLNSLPSSIAS |
| | VFLPPADLSDVPSTARIETRAMLTMTRSNPALRELFGSLSTKKSLPAVLVVDMFGADAFDVAV |
| | DFHGSPYIFYASNANVLSFFLHLPKLDKTVSCEFRYLTEPLKIPGCVPITGKDFLDTVQDRNDDAY |
| | KLLLHNTKRYKEAKGILVNSFVDLESNAIKALQEPAPDKPTVYPIGPLVNTSSSNVNLEDKFGCLS |
| | WLDNQPFGSVLYISFGSGGTLTCEQFNELAIGLAESGKRFIWVIRSPSEIVSSSYFNPHSETDPFS |
| | FLPIGFLDRTKEKGLVVPSWAPQVQILAHPSTCGFLTHCGWNSTLESIVNGVPLIAWPLFAEQK |
| | MNTLLLVEDVGAALRIHAGEDGIVRREEVVRVVKALMEGEEGKAIGNKVKELKEGVVRVLGDD |
| | GLSSKSFGEVLLKWKTHQRDINQETSH |
| SEQ ID NO: 29 | ATGGCAGATGGAAACACTCCACATGTAGCAATCATACCAAGTCCCGGTATAGGTCACCTCA |
| | TCCCACTCGTCGAGTTAGCAAAGCGACTCCTTGACAATCACGGTTTCACCGTCACTTTCATC |
| | ATCCCCGGCGATTCTCCTCCGTCTAAGGCTCAAAGATCCGTTCTCAACTCTCTCCCTTCCTCC |
| | ATAGCCTCCGTCTTCCTCCCTCCCGCCGATCTTTCCGACGTTCCTTCGACAGCTCGAATCGA |
| | AACTCGGATATCGCTCACCGTGACTCGTTCCAACCCGGCGCTCCGGGAGCTTTTTGGCTCG |
| | TTATCGGCGGAGAAACGTCTCCCGGCGGTTCTCGTCGTCGATCTATTTGGTACGGATGCGT |
| | TCGACGTGGCTGCTGAGTTCCACGTGTCGCCATACATTTTCTATGCATCAAATGCCAACGTC |
| | CTCACGTTTCTGCTTCACTTGCCGAAGCTAGACGAAACGGTGTCGTGTGAGTTTAGGGAAT |
| | TAACCGAACCGGTTATTATTCCCGGTTGTGTCCCCATAACCGGTAAGGATTTCGTCGATCC |
| | GTGTCAAGACCGAAAAGATGAATCATACAAATGGCTTCTACACAACGTCAAGAGATTCAA |
| | AGAAGCTGAAGGGATTCTAGTGAATTCCTTCGTCGATTTAGAGCCAAACACTATAAAGATT |
| | GTACAAGAACCGGCTCCTGATAAACCACCGGTTTACCTGATTGGGCCGTTGGTTAACTCGG |
| | GTTCACACGATGCTGACGTGAACGATGAGTACAAATGTTTAAATTGGCTAGACAACCAACC |
| | ATTCGGGTCGGTTCTATACGTATCCTTTGGAAGCGGCGGAACACTCACGTTTGAGCAGTTC |
| | ATGAGCTGGCTCTTGGCCTAGCGGAGAGTGGAAAACGGTTTCTTTGGGTCATACGAAGT |
| | CCGAGTGGGATAGCTAGTTCATCGTATTTCAATCCACAAAGCCGAAATGATCCATTTTCGTT |
| | TTTACCACAAGGCTTCTTAGACCGAACCAAAGAAAAAGGTCTAGTGGTTGGGTCATGGGC |
| | TCCACAGGCTCAAATTCTGACTCATACATCTATAGGTGGATTTTTAACTCATTGTGGATGGA |
| | ATTCGAGTCTAGAAAGTATTGTAAACGGTGTACCGCTCATAGCATGGCCGTTATACGCGGA |
| | GCAAAAGATGAACGCATTGCTACTCGTGGATGTTGGTGCGGCTCTAAGAGCACGACTGGG |
| | TGAAGACGGGGTCGTAGGAAGGGAAGAAGTGGCGAGAGTGGTAAAAGGATTGATAGAA |
| | GGAGAAGAAGGGAATGCGGTAAGGAAAAAAATGAAAGAGTTGAAAGAAGGATCTGTTA |
| | GAGTCTTAAGGGACGATGGATTCTCTACCAAATCGCTTAATGAAGTTTCGTTGAAGTGGAA |
| | AGCCCACCAACGAAAGATCGACCAAGAACAGGAATCATTTCTATGA |
| SEQ ID NO: 30 | MADGNTPHVAIIPSPGIGHLIPLVELAKRLLDNHGFTVTFIIPGDSPPSKAQRSVLNSLPSSIASVF |
| | LPPADLSDVPSTARIETRISLTVTRSNPALRELFGSLSAEKRLPAVLVVDLFGTDAFDVAAEFHVS |
| | PYIFYASNANVLTFLLHLPKLDETVSCEFRELTEPVIIPGCVPITGKDFVDPCQDRKDESYKWLLH |
| | NVKRFKEAEGILVNSFVDLEPNTIKIVQEPAPDKPPVYLIGPLVNSGSHDADVNDEYKCLNWLD |
| | NQPFGSVLYVSFGSGGTLTFEQFIELALGLAESGKRFLWVIRSPSGIASSSYFNPQSRNDPFSFLP |
| | QGFLDRTKEKGLVVGSWAPQAQILTHTSIGGFLTHCGWNSSLESIVNGVPLIAWPLYAEQKM |
| | NALLLVDVGAALRARLGEDGVVGREEVARVVKGLIEGEEGNAVRKKMKELKEGSVRVLRDDG |
| | FSTKSLNEVSLKWKAHQRKIDQEQESFL |
| SEQ ID NO: 31 | ATGGACCAGCCTCACGCGCTTCTAGTGGCTAGCCCTGGCTTGGGTCACCTCATCCCTATCCT |
| | GGAGCTCGGCAACCGTCTCTCCTCCGTCCTAAACATCCACGTCACCATTCTCGCGGTCACCT |
| | CCGGCTCCTCTTCACCGACAGAAACCGAAGCCATACATGCAGCCGCGGCTAGAACAATCTG |
| | TCAAATTACGGAAATTCCCTCGGTGGATGTAGACAACCTCGTGGAGCCAGATGCTACAATT |
| | TTCACTAAGATGGTGGTGAAGATGCGAGCCATGAAGCCCGCGGTACGAGATGCCGTGAA |
| | ATTAATGAAACGAAAACCAACGGTCATGATTGTTGACTTTTTGGGTACGGAACTGATGTCC |
| | GTAGCCGATGACGTAGGCATGACGGCTAAATACGTTTACGTTCCAACTCATGCGTGGTTCT |
| | TGGCAGTCATGGTGTACTTGCCGGTGTTAGATACGGTAGTGGAAGGTGAGTATGTTGATA |
| | TTAAGGAGCCTTTGAAGATACCGGGTTGTAAACCGGTCGGACCGAAGGAGCTGATGGAA |
| | ACGATGTTAGACCGGTCGGGCCAGCAATATAAAGAGTGTGTACGAGCTGGCTTAGAGGTA |
| | CCTATGAGCGATGGTGTTTTGGTAAATACTTGGGAGGAGTTACAAGGAAACACTCTCGCT |
| | GCGCTTAGAGAGGACGAAGAATTGAGCCGGGTCATGAAAGTACCGGTTTATCCTATTGGG |
| | CCAATTGTTAGGACTAACCAGCATGTAGACAAACCCAATAGTATATTCGAGTGGCTAGACG |
| | AGCAACGGGAAAGGTCAGTGGTGTTTGTGTGTTTAGGGAGCGGTGGAACGTTGACGTTT |
| | GAGCAAACAGTGGAACTCGCTTTGGGTTTAGAGTTAAGTGGTCAAAGGTTCGTTTGGGTT |
| | CTACGTAGGCCCGCTTCATATCTCGGGGCGATCTCCAGCGATGATGAACAGGTAAGTGCC |
| | AGTCTACCTGAAGGTTTCTTGGACCGCACGCGTGGTGTGGGGATTGTGGTTACGCAATGG |
| | GCACCACAAGTTGAGATCTTGAGCCATAGATCGATCGGTGGGTTCTTGTCTCACTGCGGTT |
| | GGAGTTCGGCTTTGGAAAGTTTGACTAAAGGAGTTCCGATCATCGCTTGGCCTCTTTATGC |
| | GGAGCAGTGGATGAATGCCACGTTATTGACTGAGGAGATCGGTGTGGCCGTTCGTACATC |
| | GGAGTTACCGTCGGAGAGAGTCATCGGAAGGGAAGAAGTGGCATCTCTGGTGAGAAAGA |
| | TTATGGCGGAAGAGGATGAAGAAGGACAGAAAATTAGGGCTAAAGCTGAGGAGGTGAG |
| | GGTTAGCTCCGAACGAGCTTGGAGTAAAGACGGGTCATCTTATAATTCTCTATTCGAATGG |
| | GCAAAACGATGTTATCTTGTACCCTAG |
| SEQ ID NO: 32 | MDQPHALLVASPGLGHLIPILELGNRLSSVLNIHVTILAVTSGSSSPTETEAIHAAAARTICQITEIP |
| | SVDVDNLVEPDATIFTKMVVKMRAMKPAVRDAVKLMKRKPTVMIVDFLGTELMSVADDVG |
| | MTAKYVYVPTHAWFLAVMVYLPVLDTVVEGEYVDIKEPLKIPGCKPVGPKELMETMLDRSGQ |
| | QYKECVRAGLEVPMSDGVLVNTWEELQGNTLAALREDEELSRVMKVPVYPIGPIVRTNQHVD |
| | KPNSIFEWLDEQRERSVVFVCLGSGGTLTFEQTVELALGLELSGQRFVWVLRRPASYLGAISSD |
| | DEQVSASLPEGFLDRTRGVGIVVTQWAPQVEILSHRSIGGFLSHCGWSSALESLTKGVPIIAWP |
| | LYAEQWMNATLLTEEIGVAVRTSELPSERVIGREEVASLVRKIMAEEDEEGQKIRAKAEEVRVSS |
| | ERAWSKDGSSYNSLFEWAKRCYLVP |
| SEQ ID NO: 33 | ATGCATATCACAAAACCACACGCCGCCATGTTTTCCAGTCCCGGAATGGGCCATGTCATCC |
| | CGGTGATCGAGCTTGGAAAGCGTCTCTCCGCTAACAACGGCTTCCACGTCACCGTCTTCGT |
| | CCTCGAAACCGACGCAGCCTCCGCTCAATCCAAGTTCCTAAACTCAACCGGCGTCGACATC |
| | GTCAAACTTCCATCGCCGGACATTTATGGTTTAGTGGACCCCGACGACCATGTAGTGACCA |
| | AGATCGGAGTCATTATGCGTGCAGCAGTTCCAGCCCTCCGATCCAAGATCGCTGCCATGCA |
| | TCAAAAGCCAACGGCTCTGATCGTTGACTTGTTTGGCACAGATGCGTTATGTCTCGCAAAG |
| | GAATTTAACATGTTGAGTTATGTGTTTATCCCTACCAACGCACGTTTTCTCGGAGTTTCGAT |
| | TTATTATCCAAATTTGGACAAAGATATCAAGGAAGAGCACACAGTGCAAAGAAACCCACTC |
| | GCTATACCGGGGTGTGAACCGGTTAGGTTCGAAGATACTCTGGATGCATATCTGGTTCCCG |
| | ACGAACCGGTGTACCGGGATTTTGTTCGTCATGGTCTGGCTTACCCAAAAGCCGATGGAAT |
| | TTTGGTAAATACATGGGAAGAGATGGAGCCCAAATCATTGAAGTCCCTTCTAAACCCAAAG |
| | CTCTTGGGCCGGGTTGCTCGTGTACCGGTCTATCCAATCGGTCCCTTATGCAGACCGATAC |
| | AATCATCCGAAACCGATCACCCGGTTTTGGATTGGTTAAACGAACAACCGAACGAGTCGGT |
| | TCTCTATATCTCCTTCGGGAGTGGTGGTTGTCTATCGGCGAAACAGTTAACTGAATTGGCG |
| | TGGGGACTCGAGCAGAGCCAGCAACGGTTCGTATGGGTGGTTCGACCACCGGTCGACGG |
| | TTCGTGTTGTAGCGAGTATGTCTCGGCTAACGGTGGTGGAACCGAAGACAACACGCCAGA |
| | GTATCTACCGGAAGGGTTCGTGAGTCGTACTAGTGATAGAGGTTTCGTGGTCCCCTCATGG |
| | GCCCCACAAGCTGAAATCCTGTCCCATCGGGCCGTTGGTGGGTTTTTGACCCATTGCGGTT |
| | GGAGCTCGACGTTGGAAAGCGTCGTTGGCGGCGTTCCGATGATCGCATGGCCACTTTTTG |
| | CCGAGCAGAATATGAATGCGGCGTTGCTCAGCGACGAACTGGGAATCGCAGTCAGATTGG |
| | ATGATCCAAAGGAGGATATTTCTAGGTGGAAGATTGAGGCGTTGGTGAGGAAGGTTATG |
| | ACTGAGAAGGAAGGTGAAGCGATGAGAAGGAAAGTGAAGAAGTTGAGAGACTCGGCGG |
| | AGATGTCACTGAGCATTGACGGTGGTGGTTTGGCGCACGAGTCGCTTTGCAGAGTCACCA |
| | AGGAGTGTCAACGGTTTTTGGAACGTGTCGTGGACTTGTCACGTGGTGCTTAG |
| SEQ ID NO: 34 | MHITKPHAAMFSSPGMGHVIPVIELGKRLSANNGFHVTVFVLETDAASAQSKFLNSTGVDIVK |
| | LPSPDIYGLVDPDDHVVTKIGVIMRAAVPALRSKIAAMHQKPTALIVDLFGTDALCLAKEFNML |
| | SYVFIPTNARFLGVSIYYPNLDKDIKEEHTVQRNPLAIPGCEPVRFEDTLDAYLVPDEPVYRDFVR |
| | HGLAYPKADGILVNTWEEMEPKSLKSLLNPKLLGRVARVPVYPIGPLCRPIQSSETDHPVLDWL |
| | NEQPNESVLYISFGSGGCLSAKQLTELAWGLEQSQQRFVWVVRPPVDGSCCSEYVSANGGGT |
| | EDNTPEYLPEGFVSRTSDRGFVVPSWAPQAEILSHRAVGGFLTHCGWSSTLESVVGGVPMIA |
| | WPLFAEQNMNAALLSDELGIAVRLDDPKEDISRWKIEALVRKVMTEKEGEAMRRKVKKLRDS |
| | AEMSLSIDGGGLAHESLCRVTKECQRFLERVVDLSRGA |
| SEQ ID NO: 35 | ATGGAAAAAACACCCCATATAGCTATTGTACCAAGTCCAGGAATGGGACACTTGATCCCTT |
| | TGGTTGAATTTGCCAAAAGATTGAAGAACAACCACAACATCGATGCAACTTTCATCATTCC |
| | AAATGATGGACCTCTATCCAAATCTCAACGTGTTTATCTCGATTCACTCCCAACCGGATTAA |
| | ACCATATCATTCTCCCTCCAGTTAGTTTCGATGATCTACCACAAGATGCAAAGATGGAAACC |
| | CGAATCAGCCTCATGGTTACACGATCTATCGATTTCCTTCGAGAAGCTTTGAAGTCATTAGT |
| | TGCAGAAACAAACATGGTGGCACTGTTTATTGATCTTTTTGGTACAGATGCATTTGATGTT |
| | GCTATTGAATTTGGTGTTTCACCATATGTCTTCTTTCCATCAACTGCAATGGCTTTATCTTTG |
| | TTTCTTCATTTACCAAAACTTGATCAAATGGTTTCATGTGAGTATAGGGACTTGCCTGAACC |
| | GGTTCAGATCCCGGGTTGCATACCAGTTCCCGGTCGAGACCTACTTGACCCGGTTCAAGAT |
| | AGAAAGAACGAAGCGTATAAGTGGGTGCTTCATAACGCAAAGAGGTATTCGATGGCTGA |
| | GGGTATAGCGGTAAATAGCTTCAAGGAGTTAGAAGGTGGAGCCTTGAAAGCTTTACTAGA |
| | GGAAGAACCGGGCAAACCAAAGGTTTATCCGGTTGGACCGTTGATACAGACCGGTTCAAG |
| | TACTGATGTTGATGGGTCCGAGTGTTTGAGGTGGTTAGACGGTCAGCCATGTGGTTCTGTT |
| | TTGTACGTATCTTTTGGAAGTGGTGGAACCTTATCTTCTAATCAGCTCAATGAGTTAGCCTT |
| | TGGTTTGGAATTAAGTGAGCAAAGGTTCATATGGGTGGTTAGAAGCCCGAATGATCAACC |
| | CAACGCGACTTACTTTAACTCACATGGTCATATGGACCCGTTGGGTTTCTTACCAGAAGGG |
| | TTTCTAGAAAGAACCAAAGGTTTTGGGCTTGTGGTTCCTTCTTGGGCCCCACAAGCCCAAA |
| | TCTTGAGTCATAGTTCAACCGGTGGGTTTTTAACCCACTGTGGTTGGAACTCGATTCTTGAG |
| | ACTGTAGTCCATGGTGTGCCGGTTATCGCCTGGCCACTTTACGCAGAGCAGAGGATGAAC |
| | GCGGTATCTTTAACCGAGGGTATAAAAGTGGCGTTAAGGCCCAACGTGGACGAAAATGGC |
| | ATCGTGGGCCGTGTGGAGATTGCGAGGGTCGTGAAGGGTTTGTTAGAAGGGGAAGAAG |
| | GAAAACCGATTAGGAGTCGAATTCGGGATCTTAAAGATGCAGCTGCTAATGTTCTTAGTAA |
| | AGATGGGTGTTCCACAAAAACTTTAGTGCAGTTGGCTTCCAAGTTGAAAACGAAGAGTAA |
| | ATTAAGCATTTAA |
| SEQ ID NO: 36 | MEKTPHIAIVPSPGMGHLIPLVEFAKRLKNNHNIDATFIIPNDGPLSKSQRVYLDSLPTGLNHIIL |
| | PPVSFDDLPQDAKMETRISLMVTRSIDFLREALKSLVAETNMVALFIDLFGTDAFDVAIEFGVSP |
| | YVFFPSTAMALSLFLHLPKLDQMVSCEYRDLPEPVQIPGCIPVPGRDLLDPVQDRKNEAYKWV |
| | LHNAKRYSMAEGIAVNSFKELEGGALKALLEEEPGKPKVYPVGPLIQTGSSTDVDGSECLRWLD |
| | GQPCGSVLYVSFGSGGTLSSNQLNELAFGLELSEQRFIWVVRSPNDQPNATYFNSHGHMDPL |
| | GFLPEGFLERTKGFGLVVPSWAPQAQILSHSSTGGFLTHCGWNSILETVVHGVPVIAWPLYAE |
| | QRMNAVSLTEGIKVALRPNVDENGIVGRVEIARVVKGLLEGEEGKPIRSRIRDLKDAAANVLSK |
| | DGCSTKTLVQLASKLKTKSKLSI |
| SEQ ID NO: 37 | ATGAACAGAGAAGTCTCTGAGAGAATTCATATTTTGTTCTTCCCCTTCATGGCTCAAGGCCA |
| | CATGATTCCAATTTTGGACATGGCCAAGCTTTTCTCGAGGAGAGGAGCCAAGTCAACCCTT |
| | CTCACAACCCCAATCAACGCTAAGATCTTCGAGAAACCTATTGAAGCATTCAAAAATCAAA |
| | ACCCTGATCTCGAAATCGGAATCAAGATCTTCAATTTCCCTTGTGTAGAGCTTGGATTGCCT |
| | GAAGGATGCGAGAACGCTGACTTTATCAACTCATACCAAAAATCTGACTCAGGTGACTTGT |
| | TCTTGAAGTTTCTTTTCTCTACCAAGTATATGAAACAACAGTTGGAGAGTTTCATTGAAACA |
| | ACCAAACCAAGTGCTCTTGTTGCCGATATGTTCTTCCCTTGGGCGACAGAATCTGCTGAGA |
| | AGCTCGGTGTACCAAGACTTGTGTTCCACGGTACATCTTTCTTTTCTTTGTGTTGTTCGTATA |
| | ACATGAGGATTCATAAGCCACACAAGAAAGTCGCTACGAGTTCTACTCCTTTTGTAATCCCT |
| | GGTCTCCCAGGAGACATAGTTATTACAGAAGACCAAGCCAATGTTGCCAAAGAAGAAACG |
| | CCAATGGGAAAGTTTATGAAAGAGGTTAGGGAATCAGAGACCAATAGCTTTGGTGTATTG |
| | GTTAATAGCTTCTACGAGCTGGAATCAGCTTATGCTGATTTTTATCGTAGTTTTGTGGCGAA |
| | AAGAGCTTGGCATATCGGTCCGCTTTCGCTATCTAACAGAGAGTTAGGAGAGAAAGCCAG |
| | AAGAGGGAAAAAGGCTAACATTGATGAGCAAGAATGCCTAAAATGGCTGGACTCTAAGA |
| | CACCTGGTTCAGTAGTTTACTTGTCCTTTGGGAGCGGAACTAATTTCACCAACGACCAGCT |
| | GTTAGAGATCGCTTTTGGTCTTGAAGGTTCTGGACAAAGTTTCATCTGGGTGGTTAGGAAA |
| | AATGAAAACCAAGGTGACAATGAAGAGTGGTTGCCTGAAGGGTTTAAAGAGAGGACAAC |
| | AGGGAAAGGGCTAATAATACCTGGATGGGCGCCGCAAGTGCTGATACTTGACCATAAAGC |
| | AATTGGAGGATTTGTGACTCATTGCGGATGGAACTCGGCTATAGAGGGCATTGCCGCGGG |
| | GCTGCCTATGGTAACATGGCCAATGGGGGCAGAACAGTTCTACAATGAGAAGCTATTGAC |
| | AAAAGTGTTGAGAATAGGAGTGAACGTTGGAGCTACCGAGTTGGTGAAAAAAGGAAAGT |
| | TGATTAGTAGAGCACAAGTGGAGAAGGCAGTAAGGGAAGTGATTGGTGGTGAGAAGGC |
| | AGAGGAAAGGCGGCTATGGGCTAAGAAGCTGGGCGAGATGGCTAAAGCCGCTGTGGAA |
| | GAAGGAGGGTCCTCTTATAATGATGTGAACAAGTTTATGGAAGAGCTGAATGGTAGAAAG |
| | TAG |
| SEQ ID NO: 38 | MNREVSERIHILFFPFMAQGHMIPILDMAKLFSRRGAKSTLLTTPINAKIFEKPIEAFKNQNPDL |
| | EIGIKIFNFPCVELGLPEGCENADFINSYQKSDSGDLFLKFLFSTKYMKQQLESFIETTKPSALVAD |
| | MFFPWATESAEKLGVPRLVFHGTSFFSLCCSYNMRIHKPHKKVATSSTPFVIPGLPGDIVITEDQ |
| | ANVAKEETPMGKFMKEVRESETNSFGVLVNSFYELESAYADFYRSFVAKRAWHIGPLSLSNREL |
| | GEKARRGKKANIDEQECLKWLDSKTPGSVVYLSFGSGTNFTNDQLLEIAFGLEGSGQSFIWVVR |
| | KNENQGDNEEWLPEGFKERTTGKGLIIPGWAPQVLILDHKAIGGFVTHCGWNSAIEGIAAGLP |
| | MVTWPMGAEQFYNEKLLTKVLRIGVNVGATELVKKGKLISRAQVEKAVREVIGGEKAEERRL |
| | WAKKLGEMAKAAVEEGGSSYNDVNKFMEELNGRK |
| SEQ ID NO: 39 | ATGGAGGAAAAGCCTGCAAGGAGAAGCGTAGTGTTGGTTCCATTTCCAGCACAAGGACAT |
| | ATATCTCCAATGATGCAACTTGCCAAAACCCTTCACTTAAAGGGTTTCTCGATCACAGTTGT |
| | TCAGACTAAGTTCAATTACTTTAGCCCTTCAGATGACTTCACTCATGATTTTCAGTTCGTCAC |
| | CATTCCAGAAAGCTTACCAGAGTCTGATTTCAAGAATCTCGGACCAATACAGTTTCTGTTTA |
| | AGCTCAACAAAGAGTGTAAGGTGAGCTTCAAGGACTGTTTGGGTCAGTTGGTGCTGCAAC |
| | AAAGTAATGAGATCTCATGTGTCATCTACGATGAGTTCATGTACTTTGCTGAAGCTGCAGC |
| | CAAAGAGTGTAAGCTTCCAAACATCATTTTCAGCACAACAAGTGCCACGGCTTTCGCTTGC |
| | CGCTCTGTATTTGACAAACTATATGCAAACAATGTCCAAGCTCCCTTGAAAGAAACTAAAG |
| | GACAACAAGAAGAGCTAGTTCCGGAGTTTTATCCCTTGAGATATAAAGACTTTCCAGTTTC |
| | ACGGTTTGCATCATTAGAGAGCATAATGGAGGTGTATAGGAATACAGTTGACAAACGGAC |
| | AGCTTCCTCGGTGATAATCAACACTGCGAGCTGTCTAGAGAGCTCATCTCTGTCTTTTCTGC |
| | AACAACAACAGCTACAAATTCCAGTGTATCCTATAGGCCCTCTTCACATGGTGGCCTCAGCT |
| | CCTACAAGTCTGCTTGAAGAGAACAAGAGCTGCATCGAATGGTTGAACAAACAAAAGGTA |
| | AACTCGGTGATATACATAAGCATGGGAAGCATAGCTTTAATGGAAATCAACGAGATAATG |
| | GAAGTCGCGTCAGGATTGGCTGCTAGCAACCAACACTTCTTATGGGTGATCCGACCAGGG |
| | TCAATACCTGGTTCCGAGTGGATAGAGTCCATGCCTGAAGAGTTTAGTAAGATGGTTTTGG |
| | ACCGAGGTTACATTGTGAAATGGGCTCCACAGAAGGAAGTACTTTCTCATCCTGCAGTAGG |
| | AGGGTTTTGGAGCCATTGTGGATGGAACTCGACACTAGAAAGCATCGGCCAAGGAGTTCC |
| | AATGATCTGCAGGCCATTTTCGGGTGATCAAAAGGTGAACGCTAGATACTTGGAGTGTGT |
| | ATGGAAAATTGGGATTCAAGTGGAGGGTGAGCTAGACAGAGGAGTGGTCGAGAGAGCT |
| | GTGAAGAGGTTAATGGTTGACGAAGAAGGAGAGGAGATGAGGAAGAGAGCTTTCAGTTT |
| | AAAAGAGCAACTTAGAGCCTCTGTTAAAAGTGGAGGCTCTTCACACAACTCGCTAGAAGA |
| | GTTTGTACACTTCATAAGGACTGCCTAG |
| SEQ ID NO: 40 | MEEKPARRSVVLVPFPAQGHISPMMQLAKTLHLKGFSITVVQTKFNYFSPSDDFTHDFQFVTIP |
| | ESLPESDFKNLGPIQFLFKLNKECKVSFKDCLGQLVLQQSNEISCVIYDEFMYFAEAAAKECKLPN |
| | IIFSTTSATAFACRSVFDKLYANNVQAPLKETKGQQEELVPEFYPLRYKDFPVSRFASLESIMEVY |
| | RNTVDKRTASSVIINTASCLESSSLSFLQQQQLQIPVYPIGPLHMVASAPTSLLEENKSCIEWLNK |
| | QKVNSVIYISMGSIALMEINEIMEVASGLAASNQHFLWVIRPGSIPGSEWIESMPEEFSKMVLD |
| | RGYIVKWAPQKEVLSHPAVGGFWSHCGWNSTLESIGQGVPMICRPFSGDQKVNARYLECVW |
| | KIGIQVEGELDRGVVERAVKRLMVDEEGEEMRKRAFSLKEQLRASVKSGGSSHNSLEEFVHFIR |
| | TA |
| SEQ ID NO: 41 | ATGACCAAACCCTCCGACCCAACCAGAGACTCCCACGTGGCAGTTCTCGCTTTTCCTTTCGG |
| | CACTCATGCAGCTCCTCTCCTCACCGTCACGCGCCGCCTCGCCTCCGCCTCTCCTTCCACCGT |
| | CTTCTCTTTCTTCAACACCGCACAATCCAACTCTTCGTTATTTTCCTCCGGTGACGAAGCAGA |
| | TCGTCCGGCGAACATCAGAGTATACGATATTGCCGACGGTGTTCCGGAGGGATACGTGTT |
| | TAGCGGGAGACCACAGGAGGCGATCGAGCTGTTTCTTCAAGCTGCGCCGGAGAATTTCCG |
| | GAGAGAAATCGCGAAGGCGGAGACGGAGGTTGGTACGGAAGTGAAATGTTTGATGACTG |
| | ATGCGTTCTTCTGGTTCGCGGCTGATATGGCGACGGAGATAAATGCGTCGTGGATTGCGTT |
| | TTGGACCGCCGGAGCAAACTCACTCTCTGCTCATCTCTACACAGATCTCATCAGAGAAACC |
| | ATCGGTGTCAAAGAAGTAGGTGAGCGTATGGAGGAGACAATAGGGGTTATCTCAGGAAT |
| | GGAGAAGATCAGAGTCAAAGATACACCAGAAGGAGTTGTGTTTGGGAATTTAGACTCTGT |
| | TTTCTCAAAGATGCTTCATCAAATGGGTCTTGCTTTGCCTCGTGCCACTGCTGTTTTCATCAA |
| | TTCTTTTGAAGATTTGGATCCTACATTGACGAATAACCTCAGATCGAGATTTAAACGATATC |
| | TGAACATCGGTCCTCTCGGGTTATTATCTTCTACATTGCAACAACTAGTGCAAGATCCTCAC |
| | GGTTGTTTGGCTTGGATGGAGAAGAGATCTTCTGGTTCTGTGGCGTACATTAGCTTTGGTA |
| | CGGTCATGACACCGCCTCCTGGAGAGCTTGCGGCGATAGCAGAAGGGTTGGAATCGAGTA |
| | AAGTGCCGTTTGTTTGGTCGCTTAAGGAGAAGAGCTTGGTTCAGTTACCAAAAGGGTTTTT |
| | GGATAGGACAAGAGAGCAAGGGATAGTGGTTCCATGGGCACCGCAAGTGGAACTGCTGA |
| | AACACGAAGCAACGGGTGTGTTTGTGACGCATTGTGGATGGAACTCGGTGTTGGAGAGT |
| | GTATCGGGTGGTGTACCGATGATTTGCAGGCCATTTTTTGGGGATCAGAGATTGAACGGA |
| | AGAGCGGTGGAGGTTGTGTGGGAGATTGGAATGACGATTATCAATGGAGTCTTCACGAA |
| | AGATGGGTTTGAGAAGTGTTTGGATAAAGTTTTAGTTCAAGATGATGGTAAGAAGATGAA |
| | ATGTAATGCTAAGAAACTTAAAGAACTAGCTTACGAAGCTGTCTCTTCTAAAGGAAGGTCC |
| | TCTGAGAATTTCAGAGGATTGTTGGATGCAGTTGTAAACATTATCTAG |
| SEQ ID NO: 42 | MTKPSDPTRDSHVAVLAFPFGTHAAPLLTVTRRLASASPSTVFSFFNTAQSNSSLFSSGDEADR |
| | PANIRVYDIADGVPEGYVFSGRPQEAIELFLQAAPENFRREIAKAETEVGTEVKCLMTDAFFWF |
| | AADMATEINASWIAFWTAGANSLSAHLYTDLIRETIGVKEVGERMEETIGVISGMEKIRVKDTP |
| | EGVVFGNLDSVFSKMLHQMGLALPRATAVFINSFEDLDPTLTNNLRSRFKRYLNIGPLGLLSSTL |
| | QQLVQDPHGCLAWMEKRSSGSVAYISFGTVMTPPPGELAAIAEGLESSKVPFVWSLKEKSLVQ |
| | LPKGFLDRTREQGIVVPWAPQVELLKHEATGVFVTHCGWNSVLESVSGGVPMICRPFFGDQR |
| | LNGRAVEVVWEIGMTIINGVFTKDGFEKCLDKVLVQDDGKKMKCNAKKLKELAYEAVSSKGRS |
| | SENFRGLLDAVVNII |
| SEQ ID NO: 43 | ATGAAAGTGAACGAGGAAAACAACAAGCCGACAAAGACCCATGTCTTAATCTTCCCATTTC |
| | CGGCGCAAGGTCACATGATTCCCCTCCTCGACTTCACCCACCGCCTTGCTCTCCGCGGCGG |
| | CGCCGCCTTAAAAATAACCGTCCTAGTCACTCCAAAAAACCTTCCTTTTCTCTCTCCGCTTCT |
| | CTCCGCCGTAGTTAACATCGAACCACTTATCCTCCCTTTTCCCTCCCACCCTTCAATCCCCTC |
| | CGGCGTCGAAAACGTCCAAGACTTACCTCCTTCAGGCTTCCCTTTAATGATCCACGCGCTTG |
| | GTAATCTCCACGCGCCGCTTATCTCTTGGATTACTTCTCACCCTTCTCCTCCAGTAGCCATCG |
| | TATCTGATTTCTTCCTTGGTTGGACCAAAAACCTCGGAATCCCTCGTTTCGATTTCTCTCCCT |
| | CCGCTGCTATCACTTGCTGCATACTCAATACTCTCTGGATCGAAATGCCCACCAAGATCAAC |
| | GAAGATGACGATAACGAGATCCTCCACTTTCCCAAGATCCCGAATTGTCCAAAATACCGTT |
| | TTGATCAGATCTCCTCTCTTTACAGAAGTTACGTTCACGGAGATCCAGCTTGGGAGTTCATA |
| | AGAGACTCCTTTAGAGATAACGTGGCGAGTTGGGGACTCGTCGTGAACTCGTTCACCGCC |
| | ATGGAAGGTGTTTATCTCGAACATCTTAAGCGAGAGATGGGCCATGATCGTGTATGGGCT |
| | GTAGGCCCAATTATTCCGTTATCTGGGGATAACCGTGGTGGCCCGACTTCTGTTTCTGTTG |
| | ATCACGTGATGTCGTGGCTTGACGCACGTGAGGATAACCACGTGGTGTACGTGTGCTTTG |
| | GAAGTCAAGTAGTTTTGACTAAAGAGCAGACTCTTGCACTCGCCTCTGGGCTTGAGAAAA |
| | GCGGCGTCCATTTCATATGGGCCGTAAAGGAGCCCGTTGAGAAAGACTCAACACGTGGCA |
| | ACATCCTGGACGGTTTCGACGATCGCGTGGCTGGGAGAGGTCTGGTGATCAGAGGATGG |
| | GCTCCACAAGTAGCTGTGCTACGTCACCGAGCCGTTGGCGCGTTTTTAACGCACTGTGGTT |
| | GGAACTCTGTGGTGGAGGCGGTTGTCGCCGGCGTTTTGATGCTGACGTGGCCGATGAGA |
| | GCTGACCAGTACACTGACGCGTCTCTGGTGGTTGATGAGTTGAAAGTAGGTGTGCGTGCT |
| | TGCGAAGGACCTGACACGGTGCCTGACCCGGACGAGTTAGCTCGAGTTTTCGCTGATTCC |
| | GTGACCGGAAATCAAACGGAGAGGATCAAAGCCGTGGAGCTGAGGAAAGCAGCGTTGG |
| | ATGCGATTCAAGAACGTGGGAGCTCAGTGAATGATTTAGATGGATTTATCCAACATGTCGT |
| | TAGTTTAGGACTAAACCGCTAG |
| SEQ ID NO: 44 | MKVNEENNKPTKTHVLIFPFPAQGHMIPLLDFTHRLALRGGAALKITVLVTPKNLPFLSPLLSAV |
| | VNIEPLILPFPSHPSIPSGVENVQDLPPSGFPLMIHALGNLHAPLISWITSHPSPPVAIVSDFFLG |
| | WTKNLGIPRFDFSPSAAITCCILNTLWIEMPTKINEDDDNEILHFPKIPNCPKYRFDQISSLYRSYV |
| | HGDPAWEFIRDSFRDNVASWGLVVNSFTAMEGVYLEHLKREMGHDRVWAVGPIIPLSGDNR |
| | GGPTSVSVDHVMSWLDAREDNHVVYVCFGSQVVLTKEQTLALASGLEKSGVHFIWAVKEPVE |
| | KDSTRGNILDGFDDRVAGRGLVIRGWAPQVAVLRHRAVGAFLTHCGWNSVVEAVVAGVLM |
| | LTWPMRADQYTDASLVVDELKVGVRACEGPDTVPDPDELARVFADSVTGNQTERIKAVELRK |
| | AALDAIQERGSSVNDLDGFIQHVVSLGLNR |
| SEQ ID NO: 45 | ATGGAGTTAGAAAAAGTTCACGTGGTTTTGTTCCCATACTTGTCCAAAGGGCACATGATTC |
| | CTATGCTCCAATTAGCTCGTCTCCTCTTATCCCACTCCTTCGCCGGAGACATCTCCGTCACCG |
| | TCTTCACCACTCCTTTGAACCGTCCTTTCATCGTTGACTCACTCTCCGGCACCAAAGCGACC |
| | ATCGTCGACGTACCTTTCCCTGATAACGTCCCGGAGATCCCACCCGGCGTCGAGTGCACTG |
| | ACAAACTCCCTGCTTTGTCGTCCTCCCTCTTCGTTCCTTTCACAAGAGCCACCAAGTCAATGC |
| | AGGCAGACTTTGAGCGAGAGCTCATGTCACTGCCACGTGTCAGTTTCATGGTCTCAGACG |
| | GTTTCTTGTGGTGGACGCAAGAGTCAGCTCGAAAGCTAGGGTTTCCTCGGCTTGTTTTCTTT |
| | GGTATGAATTGCGCTTCCACCGTTATATGTGACAGTGTTTTTCAAAACCAGCTTCTATCTAA |
| | TGTTAAGTCCGAGACGGAGCCAGTTTCTGTACCGGAGTTTCCGTGGATTAAGGTTAGGAA |
| | ATGTGATTTCGTTAAAGATATGTTTGATCCAAAAACCACCACAGATCCTGGATTCAAGCTTA |
| | TCCTAGATCAAGTCACGTCTATGAATCAAAGCCAAGGTATCATATTCAATACATTTGACGAC |
| | CTTGAACCCGTGTTTATTGATTTCTACAAGCGTAAACGCAAACTCAAGCTTTGGGCAGTTG |
| | GACCGCTTTGTTACGTAAATAACTTCTTGGATGATGAAGTAGAAGAGAAGGTCAAACCTA |
| | GTTGGATGAAATGGCTAGATGAAAAGCGAGACAAGGGATGCAATGTTCTGTATGTGGCTT |
| | TCGGGTCACAAGCCGAGATCTCGAGAGAACAACTAGAGGAGATTGCGTTAGGGTTGGAA |
| | GAATCGAAGGTGAACTTCTTGTGGGTGGTCAAAGGAAATGAAATAGGAAAAGGGTTTGA |
| | AGAGAGAGTGGGAGAAAGAGGAATGATGGTGAGAGATGAATGGGTTGATCAGAGGAAG |
| | ATATTAGAGCACGAGAGTGTTAGAGGGTTCTTGAGCCATTGTGGGTGGAATTCTCTGACG |
| | GAGAGCATTTGCTCGGAGGTTCCAATCTTGGCGTTTCCTTTAGCAGCGGAGCAACCTCTGA |
| | ATGCGATTTTGGTGGTGGAAGAGCTGAGAGTGGCGGAGAGAGTGGTGGCGGCGAGTGA |
| | AGGGGTTGTGAGAAGAGAAGAGATTGCAGAGAAAGTGAAGGAGTTGATGGAGGGAGAG |
| | AAAGGGAAAGAGCTGAGGAGGAATGTCGAGGCATATGGTAAGATGGCGAAGAAGGCTT |
| | TGGAGGAAGGTATTGGTTCGTCTAGGAAGAATTTAGACAACCTTATCAACGAGTTTTGTAA |
| | CAATGGAACATGA |
| SEQ ID NO: 46 | MELEKVHVVLFPYLSKGHMIPMLQLARLLLSHSFAGDISVTVFTTPLNRPFIVDSLSGTKATIVD |
| | VPFPDNVPEIPPGVECTDKLPALSSSLFVPFTRATKSMQADFERELMSLPRVSFMVSDGFLWW |
| | TQESARKLGFPRLVFFGMNCASTVICDSVFQNQLLSNVKSETEPVSVPEFPWIKVRKCDFVKD |
| | MFDPKTTTDPGFKLILDQVTSMNQSQGIIFNTFDDLEPVFIDFYKRKRKLKLWAVGPLCYVNNF |
| | LDDEVEEKVKPSWMKWLDEKRDKGCNVLYVAFGSQAEISREQLEEIALGLEESKVNFLWVVK |
| | GNEIGKGFEERVGERGMMVRDEWVDQRKILEHESVRGFLSHCGWNSLTESICSEVPILAFPLA |
| | AEQPLNAILVVEELRVAERVVAASEGVVRREEIAEKVKELMEGEKGKELRRNVEAYGKMAKKA |
| | LEEGIGSSRKNLDNLINEFCNNGT |
| SEQ ID NO: 47 | ATGGAGCATACACCTCACATTGCTATGGTGCCCACTCCGGGAATGGGTCATCTGATCCCCC |
| | TCGTTGAGTTCGCTAAACGACTCGTCCTCCGTCACAACTTTGGCGTCACTTTTATTATCCCA |
| | ACCGATGGACCTCTCCCTAAAGCACAGAAGAGTTTTCTTGATGCTCTTCCCGCCGGCGTAA |
| | ACTATGTTCTTCTTCCCCCGGTAAGCTTCGACGACTTACCCGCTGATGTTAGGATAGAGACC |
| | CGTATTTGTCTCACCATCACTCGCTCTCTCCCGTTTGTTCGGGATGCCGTTAAGACTCTACTC |
| | GCCACCACCAAGTTAGCTGCTCTAGTGGTGGATCTTTTCGGCACCGATGCATTTGATGTTG |
| | CAATTGAGTTCAAGGTCTCCCCTTATATCTTCTATCCTACGACGGCCATGTGCCTGTCTCTTT |
| | TCTTTCACTTGCCTAAGCTTGATCAAATGGTGTCCTGCGAATATAGAGACGTCCCAGAACC |
| | ATTGCAGATTCCAGGATGCATACCCATTCACGGGAAGGATTTTCTTGACCCAGCTCAGGAT |
| | CGCAAAAATGATGCCTACAAATGCCTCCTTCACCAGGCCAAGAGATACCGGTTAGCTGAG |
| | GGTATCATGGTCAACACCTTCAACGACTTGGAGCCAGGACCCTTAAAAGCTTTGCAGGAG |
| | GAAGACCAGGGTAAGCCACCCGTTTATCCGATCGGACCACTCATCAGAGCGGATTCAAGC |
| | AGCAAGGTCGACGACTGTGAATGTTTGAAATGGCTAGATGACCAGCCACGTGGGTCGGTT |
| | CTGTTTATTTCTTTCGGAAGCGGTGGGGCAGTCTACCATAATCAGTTCATTGAGCTAGCTTT |
| | GGGATTAGAGATGAGCGAGCAAAGATTCTTGTGGGTTGTCCGAAGCCCAAATGATAAAAT |
| | TGCGAATGCAACGTATTTCAGCATTCAAAATCAGAATGATGCTCTTGCATATCTGCCAGAA |
| | GGATTCTTGGAGAGAACCAAGGGGCGTTGTCTTTTGGTCCCGTCTTGGGCGCCGCAGACT |
| | GAAATTCTTAGCCATGGTTCCACGGGTGGATTTCTAACCCACTGCGGGTGGAACTCTATTC |
| | TTGAGAGTGTAGTTAATGGGGTGCCGCTAATTGCTTGGCCTCTTTATGCAGAGCAAAAGAT |
| | GAACGCCGTAATGTTGACGGAGGGTCTTAAAGTGGCCCTGAGGCCAAAAGCCGGTGAAA |
| | ATGGCTTGATAGGCCGAGTCGAGATCGCCAATGCCGTTAAGGGCTTAATGGAGGGAGAG |
| | GAAGGAAAGAAGTTCCGCAGCACAATGAAAGACCTAAAAGATGCGGCATCGAGGGCGCT |
| | AAGTGATGACGGTTCTTCGACAAAAGCACTCGCTGAATTGGCTTGCAAGTGGGAGAACAA |
| | AATGTCCAGTACCTAG |
| SEQ ID NO: 48 | MEHTPHIAMVPTPGMGHLIPLVEFAKRLVLRHNFGVTFIIPTDGPLPKAQKSFLDALPAGVNYV |
| | LLPPVSFDDLPADVRIETRICLTITRSLPFVRDAVKTLLATTKLAALVVDLFGTDAFDVAIEFKVSPY |
| | IFYPTTAMCLSLFFHLPKLDQMVSCEYRDVPEPLQIPGCIPINGKDFLDPAQDRKNDAYKCLLH |
| | QAKRYRLAEGIMVNTFNDLEPGPLKALQEEDQGKPPVYPIGPLIRADSSSKVDDCECLKWLDD |
| | QPRGSVLFISFGSGGAVYHNQFIELALGLEMSEQRFLWVVRSPNDKIANATYFSIQNQNDALA |
| | YLPEGFLERTKGRCLLVPSWAPQTEILSHGSTGGFLTHCGWNSILESVVNGVPLIAWPLYAEQK |
| | MNAVMLTEGLKVALRPKAGENGLIGRVEIANAVKGLMEGEEGKKFRSTMKDLKDAASRALSD |
| | DGSSTKALAELACKWENKMSST |
| SEQ ID NO: 49 | ATGACTACTCAAAAAGCTCATTGCTTGATCTTACCATATCCAGCTCAGGGTCATATCAACCC |
| | TATGCTCCAATTCTCCAAACGTTTGCAATCCAAAGGTGTCAAAATCACTATAGCAGCCACCA |
| | AATCATTCTTGAAAACCATGCAAGAATTGTCAACTTCTGTGTCAGTCGAGGCTATCTCCGAT |
| | GGCTATGATGATGGCGGACGCGAGCAAGCTGGAACCTTTGTGGCCTATATTACAAGATTC |
| | AAAGAAGTTGGCTCGGATACTTTGTCTCAGCTTATTGGAAAGTTAACAAATTGTGGTTGTC |
| | CTGTGAGTTGCATAGTTTACGATCCATTTCTTCCTTGGGCTGTTGAAGTGGGAAATAATTTT |
| | GGAGTAGCTACTGCTGCTTTTTTCACTCAATCTTGTGCAGTGGATAACATTTATTACCATGT |
| | ACATAAAGGGGTTCTAAAACTTCCTCCAACTGACGTTGATAAAGAAATCTCAATTCCTGGA |
| | TTATTAACAATTGAGGCATCAGATGTACCTAGTTTTGTTTCTAATCCTGAATCTTCAAGAAT |
| | ACTTGAAATGTTGGTGAATCAGTTCTCGAATCTTGAGAACACAGATTGGGTCCTAATCAAC |
| | AGTTTCTATGAATTGGAGAAAGAGGTAATTGATTGGATGGCCAAGATCTATCCAATCAAG |
| | ACAATTGGACCAACTATACCATCAATGTACCTAGACAAGAGGCTACCAGATGACAAAGAA |
| | TATGGCCTTAGTGTCTTCAAGCCAATGACAAATGCATGCCTAAACTGGTTAAACCATCAAC |
| | CAGTTAGCTCAGTAGTATATGTATCATTTGGAAGTTTAGCCAAATTAGAAGCAGAGCAAAT |
| | GGAAGAATTAGCATGGGGTTTGAGTAATAGCAACAAGAACTTCTTGTGGGTAGTTAGATC |
| | CACTGAAGAATCCAAACTTCCCAACAACTTTTTAGAGGAATTAGCAAGTGAAAAAGGATTA |
| | GTCGTGTCATGGTGTCCACAATTACAAGTCTTGGAACATAAATCAATAGGGTGTTTTCTCA |
| | CGCACTGTGGCTGGAATTCAACTTTGGAAGCAATTAGTTTGGGAGTACCAATGATTGCAAT |
| | GCCACATTGGTCAGACCAGCCAACAAATGCGAAGCTTGTGGAAGATGTTTGGGAGATGGG |
| | AATTAGACCAAAACAAGATGAAAAAGGATTAGTTAGAAGAGAAGTTATTGAAGAATGTAT |
| | TAAGATAGTGATGGAGGAAAAGAAAGGAAAAAAGATTAGGGAAAATGCAAAGAAATGG |
| | AAGGAATTGGCTAGGAAAGCTGTGGATGAAGGAGGAAGTTCAGATAGAAATATTGAAGA |
| | ATTTGTTTCCAAGTTGGTGACTATTGCCTCAGTGGAAAGCTAA |
| SEQ ID NO: 50 | MTTQKAHCLILPYPAQGHINPMLQFSKRLQSKGVKITIAATKSFLKTMQELSTSVSVEAISDGYD |
| | DGGREQAGTFVAYITRFKEVGSDTLSQLIGKLTNCGCPVSCIVYDPFLPWAVEVGNNFGVATA |
| | AFFTQSCAVDNIYYHVHKGVLKLPPTDVDKEISIPGLLTIEASDVPSFVSNPESSRILEMLVNQFS |
| | NLENTDWVLINSFYELEKEVIDWMAKIYPIKTIGPTIPSMYLDKRLPDDKEYGLSVFKPMTNACL |
| | NWLNHQPVSSVVYVSFGSLAKLEAEQMEELAWGLSNSNKNFLWVVRSTEESKLPNNFLEELA |
| | SEKGLVVSWCPQLQVLEHKSIGCFLTHCGWNSTLEAISLGVPMIAMPHWSDQPTNAKLVEDV |
| | WEMGIRPKQDEKGLVRREVIEECIKIVMEEKKGKKIRENAKKWKELARKAVDEGGSSDRNIEEF |
| | VSKLVTIASVES |
| SEQ ID NO: 51 | ATGACTACTCACAAAGCTCATTGCTTAATTTTGCCATTTCCAGGCCAAGGTCATATCAACCC |
| | AATGCTTCAATTCTCCAAACGTTTACAATCCAAACGCGTTAAAATCACTATAGCACTCACAA |
| | AATCCTGTTTGAAAACAATGCAAGAATTGTCAACTTCAGTATCAATCGAGGCGATTTCTGA |
| | TGGCTACGATGATGGTGGTTTCCATCAAGCAGAAAATTTCGTAGCCTACATAACACGATTC |
| | AAAGAAGTTGGTTCGGATACTCTGTCTCAGCTTATTAAAAAATTGGAAAATAGTGATTGTC |
| | CTGTAAATTGCATAGTATATGATCCATTCATTCCTTGGGCTGTTGAAGTTGCAAAACAATTT |
| | GGATTAATTAGTGCTGCATTTTTCACACAAAATTGTGTAGTGGATAATCTTTATTACCATGT |
| | ACATAAAGGGGTGATAAAACTTCCACCTACTCAAAATGACGAAGAAATATTAATTCCTGGA |
| | TTTCCAAATTCGATCGATGCATCAGATGTACCTTCTTTTGTTATTAGTCCTGAAGCAGAAAG |
| | GATAGTTGAAATGTTAGCAAATCAATTCTCAAATCTTGACAAAGTTGATTATGTTCTAATCA |
| | ATAGCTTCTATGAGTTGGAGAAAGAGGTAAATGAATGGATGTCAAAGATATATCCAATAA |
| | AGACAATTGGACCAACAATACCATCAATGTACTTAGACAAGAGACTACATGATGATAAAG |
| | AGTATGGTCTTAGTGTCTTCAAGCCAATGACAAATGAATGTCTAAATTGGTTAAACCATCA |
| | ACCAATTAGCTCAGTGGTGTATGTATCATTTGGAAGTATAACCAAATTAGGAGATGAGCAA |
| | ATGGAAGAATTGGCATGGGGTTTGAAGAATAGCAACAAGAGCTTCTTGTGGGTTGTTAGG |
| | TCTACTGAAGAGCCCAAACTTCCCAACAACTTTATTGAGGAATTAACAAGTGAAAAAGGCT |
| | TAGTGGTGTCATGGTGTCCACAATTACAAGTGTTGGAACATGAATCGACAGGTTGTTTTCT |
| | GACGCACTGTGGATGGAATTCAACTCTGGAAGCGATTAGTTTGGGAGTGCCAATGGTGGC |
| | AATGCCACAATGGTCTGATCAACCAACAAATGCAAAGCTTGTGAAAGATGTTTGGGAAAT |
| | AGGTGTTAGAGCCAAACAAGATGAAAAAGGGGTAGTTAGAAGAGAAGTTATAGAAGAAT |
| | GTATAAAGCTAGTGATGGAAGAAGATAAAGGAAAACTAATTAGAGAAAATGCAAAGAAA |
| | TGGAAGGAAATAGCTAGAAATGTTGTGAATGAAGGAGGAAGTTCAGATAAAAACATTGA |
| | AGAATTTGTTTCCAAGTTGGTTACTATTTCCTAA |
| SEQ ID NO: 52 | MTTHKAHCLILPFPGQGHINPMLQFSKRLQSKRVKITIALTKSCLKTMQELSTSVSIEAISDGYDD |
| | GGFHQAENFVAYITRFKEVGSDTLSQLIKKLENSDCPVNCIVYDPFIPWAVEVAKQFGLISAAFF |
| | TQNCVVDNLYYHVHKGVIKLPPTQNDEEILIPGFPNSIDASDVPSFVISPEAERIVEMLANQFSN |
| | LDKVDYVLINSFYELEKEVNEWMSKIYPIKTIGPTIPSMYLDKRLHDDKEYGLSVFKPMTNECLN |
| | WLNHQPISSVVYVSFGSITKLGDEQMEELAWGLKNSNKSFLWVVRSTEEPKLPNNFIEELTSEK |
| | GLVVSWCPQLQVLEHESTGCFLTHCGWNSTLEAISLGVPMVAMPQWSDQPTNAKLVKDVW |
| | EIGVRAKQDEKGVVRREVIEECIKLVMEEDKGKLIRENAKKWKEIARNVVNEGGSSDKNIEEFV |
| | SKLVTIS |
| SEQ ID NO: 53 | CTGCTAACAAAGCCCGAAAGGAAGCTGAGTTGGCTGCTGCCACCGCTGAGCAATAACTAG |
| | CATAACCCCTTGGGGCCTCTAAACGGGTCTTGAGGGGTTTTTTGCTGAAAGGAGGAACTAT |
| | ATCCGGATATCCCGCAAGAGGCCCGGCAGTACCGGCATAACCAAGCCTATGCCTACAGCA |
| | TCCAGGGTGACGGTGCCGAGGATGACGATGAGCGCATTGTTAGATTTCATACACGGTGCC |
| | TGACTGCGTTAGCAATTTAACTGTGATAAACTACCGCATTAAAGCTAGCTTATCGATGATA |
| | AGCTGTCAAACATGAGAATTAATTCTTGAAGACGAAAGGGCCTCGTGATACGCCTATTTTT |
| | ATAGGTTAATGTCATGATAATAATGGTTTCTTAGACGTCAGGTGGCACTTTTCGGGGAAAT |
| | GTGCGCGGAACCCCTATTTGTTTATTTTTCTAAATACAGCTCAGTGGAACGAAAACTCACGT |
| | TAAGGGATTTTGGTCATGAGATTATCAAAAAGGATCTTCACCTAGATCCTTTTAAATTAAAA |
| | ATGAAGTTTTAAATCAATCTAAAGTATATATGAGTAAACTTGGTCTGACAGTTACCAATGCT |
| | TAATCAGTGAGGCACCTATCTCAGCGATCTGTCTATTTCGTTCATCCATAGTTGCCTGACTC |
| | CCCGTCGTGTAGATAACTACGATACGGGAGGGCTTACCATCTGGCCCCAGTGCTGCAATG |
| | ATACCGCGAGAACCACGCTCACCGGCTCCAGATTTATCAGCAATAAACCAGCCAGCCGGA |
| | AGGGCCGAGCGCAGAAGTGGTCCTGCAACTTTATCCGCCTCCATCCAGTCTATTAATTGTT |
| | GCCGGGAAGCTAGAGTAAGTAGTTCGCCAGTTAATAGTTTGCGCAACGTTGTTGCCATTGC |
| | TACAGGCATCGTGGTGTCACGCTCGTCGTTTGGTATGGCTTCATTCAGCTCCGGTTCCCAAC |
| | GATCAAGGCGAGTTACATGATCCCCCATGTTGTGCAAAAAAGCGGTTAGCTCCTTCGGTCC |
| | TCCGATCGTTGTCAGAAGTAAGTTGGCCGCAGTGTTATCACTCATGGTTATGGCAGCACTG |
| | CATAATTCTCTTACTGTCATGCCATCCGTAAGATGCTTTTCTGTGACTGGTGAGTACTCAAC |
| | CAAGTCATTCTGAGAATAGTGTATGCGGCGACCGAGTTGCTCTTGCCCGGCGTCAATACG |
| | GGATAATACCGCGCCACATAGCAGAACTTTAAAAGTGCTCATCATTGGAAAACGTTCTTCG |
| | GGGCGAAAACTCTCAAGGATCTTACCGCTGTTGAGATCCAGTTCGATGTAACCCACTCGTG |
| | CACCCAACTGATCTTCAGCATCTTTTACTTTCACCAGCGTTTCTGGGTGAGCAAAAACAGGA |
| | AGGCAAAATGCCGCAAAAAAGGGAATAAGGGCGACACGGAAATGTTGAATACTCATACT |
| | CTTCCTTTTTCAATATTATTGAAGCATTTATCAGGGTTATTGTCTCATGAGCGGATACATATT |
| | TGAAGTCAGACCCCGTAGAAAAGATCAAAGGATCTTCTTGAGATCCTTTTTTTCTGCGCGT |
| | AATCTGCTGCTGCAAACAAAAAAACCACCGCTACCAGCGGTGGTTTGTTTGCCGGATCAA |
| | GAGCTACCAACTCTTTTTCCGAAGGTAACTGGCTTCAGCAGAGCGCAGATACCAAATACTG |
| | TCCTTCTAGTGTAGCCGTAGTTAGGCCACCACTTCAAGAACTCTGTAGCACCGCCTACATAC |
| | CTCGCTCTGCTAATCCTGTTACCAGTGGCTGCTGCCAGTGGCGATAAGTCGTGTCTTACCG |
| | GGTTGGACTCAAGACGATAGTTACCGGATAAGGCGCAGCGGTCGGGCTGAACGGGGGGT |
| | TCGTGCACACAGCCCAGCTTGGAGCGAACGACCTACACCGAACTGAGATACCTACAGCGT |
| | GAGCTATGAGAAAGCGCCACGCTTCCCGAAGGGAGAAAGGCGGACAGGTATCCGGTAAG |
| | CGGCAGGGTCGGAACAGGAGAGCGCACGAGGGAGCTTCCAGGGGGAAACGCCTGGTAT |
| | CTTTATAGTCCTGTCGGGTTTCGCCACCTCTGACTTGAGCGTCGATTTTTGTGATGCTCGTC |
| | AGGGGGGCGGAGCCTATGGAAAAACGCCAGCAACGCGGCCTTTTTACGGTTCCTGGCCTT |
| | TTGCTGGCCTTTTGCTCACATGTTCTTTCCTGCGTTATCCCCTGATTCTGTGGATAACCGTAT |
| | TACCGCCTTTGAGTGAGCTGATACCGCTCGCCGCAGCCGAACGACCGAGCGCAGCGAGTC |
| | AGTGAGCGAGGAAGCGGAAGAGCGCCTGATGCGGTATTTTCTCCTTACGCATCTGTGCGG |
| | TATTTCACACCGCAATGGTGCACTCTCAGTACAATCTGCTCTGATGCCGCATAGTTAAGCCA |
| | GTATACACTCCGCTATCGCTACGTGACTGGGTCATGGCTGCGCCCCGACACCCGCCAACAC |
| | CCGCTGACGCGCCCTGACGGGCTTGTCTGCTCCCGGCATCCGCTTACAGACAAGCTGTGAC |
| | CGTCTCCGGGAGCTGCATGTGTCAGAGGTTTTCACCGTCATCACCGAAACGCGCGAGGCA |
| | GCTGCGGTAAAGCTCATCAGCGTGGTCGTGAAGCGATTCACAGATGTCTGCCTGTTCATCC |
| | GCGTCCAGCTCGTTGAGTTTCTCCAGAAGCGTTAATGTCTGGCTTCTGATAAAGCGGGCCA |
| | TGTTAAGGGCGGTTTTTTCCTGTTTGGTCACTGATGCCTCCGTGTAAGGGGGATTTCTGTTC |
| | ATGGGGGTAATGATACCGATGAAACGAGAGAGGATGCTCACGATACGGGTTACTGATGA |
| | TGAACATGCCCGGTTACTGGAACGTTGTGAGGGTAAACAACTGGCGGTATGGATGCGGC |
| | GGGACCAGAGAAAAATCACTCAGGGTCAATGCCAGCGCTTCGTTAATACAGATGTAGGTG |
| | TTCCACAGGGTAGCCAGCAGCATCCTGCGATGCAGATCCGGAACATAATGGTGCAGGGCG |
| | CTGACTTCCGCGTTTCCAGACTTTACGAAACACGGAAACCGAAGACCATTCATGTTGTTGCT |
| | CAGGTCGCAGACGTTTTGCAGCAGCAGTCGCTTCACGTTCGCTCGCGTATCGGTGATTCAT |
| | TCTGCTAACCAGTAAGGCAACCCCGCCAGCCTAGCCGGGTCCTCAACGACAGGAGCACGA |
| | TCATGCTAGTCATGCCCCGCGCCCACCGGAAGGAGCTGACTGGGTTGAAGGCTCTCAAGG |
| | GCATCGGTCGAGATCCCGGTGCCTAATGAGTGAGCTAACTTACATTAATTGCGTTGCGCTC |
| | ACTGCCCGCTTTCCAGTCGGGAAACCTGTCGTGCCAGCTGCATTAATGAATCGGCCAACGC |
| | GCGGGGAGAGGCGGTTTGCGTATTGGGCGCCAGGGTGGTTTTTCTTTTCACCAGTGAGAC |
| | GGGCAACAGCTGATTGCCCTTCACCGCCTGGCCCTGAGAGAGTTGCAGCAAGCGGTCCAC |
| | GCTGGTTTGCCCCAGCAGGCGAAAATCCTGTTTGATGGTGGTTAACGGCGGGATATAACA |
| | TGAGCTGTCTTCGGTATCGTCGTATCCCACTACCGAGATATCCGCACCAACGCGCAGCCCG |
| | GACTCGGTAATGGCGCGCATTGCGCCCAGCGCCATCTGATCGTTGGCAACCAGCATCGCA |
| | GTGGGAACGATGCCCTCATTCAGCATTTGCATGGTTTGTTGAAAACCGGACATGGCACTCC |
| | AGTCGCCTTCCCGTTCCGCTATCGGCTGAATTTGATTGCGAGTGAGATATTTATGCCAGCC |
| | AGCCAGACGCAGACGCGCCGAGACAGAACTTAATGGGCCCGCTAACAGCGCGATTTGCTG |
| | GTGACCCAATGCGACCAGATGCTCCACGCCCAGTCGCGTACCGTCTTCATGGGAGAAAAT |
| | AATACTGTTGATGGGTGTCTGGTCAGAGACATCAAGAAATAACGCCGGAACATTAGTGCA |
| | GGCAGCTTCCACAGCAATGGCATCCTGGTCATCCAGCGGATAGTTAATGATCAGCCCACTG |
| | ACGCGTTGCGCGAGAAGATTGTGCACCGCCGCTTTACAGGCTTCGACGCCGCTTCGTTCTA |
| | CCATCGACACCACCACGCTGGCACCCAGTTGATCGGCGCGAGATTTAATCGCCGCGACAAT |
| | TTGCGACGGCGCGTGCAGGGCCAGACTGGAGGTGGCAACGCCAATCAGCAACGACTGTTT |
| | GCCCGCCAGTTGTTGTGCCACGCGGTTGGGAATGTAATTCAGCTCCGCCATCGCCGCTTCC |
| | ACTTTTTCCCGCGTTTTCGCAGAAACGTGGCTGGCCTGGTTCACCACGCGGGAAACGGTCT |
| | GATAAGAGACACCGGCATACTCTGCGACATCGTATAACGTTACTGGTTTCACATTCACCAC |
| | CCTGAATTGACTCTCTTCCGGGCGCTATCATGCCATACCGCGAAAGGTTTTGCGCCATTCG |
| | ATGGTGTCCGGGATCTCGACGCTCTCCCTTATGCGACTCCTGCATTAGGAAGCAGCCCAGT |
| | AGTAGGTTGAGGCCGTTGAGCACCGCCGCCGCAAGGAATGGTGCATGCAAGGAGATGGC |
| | GCCCAACAGTCCCCCGGCCACGGGGCCTGCCACCATACCCACGCCGAAACAAGCGCTCAT |
| | GAGCCCGAAGTGGCGAGCCCGATCTTCCCCATCGGTGATGTCGGCGATATAGGCGCCAGC |
| | AACCGCACCTGTGGCGCCGGTGATGCCGGCCACGATGCGTCCGGCGTAGAGGATCGAGA |
| | TCTCGATCCCGCGAAATTAATACGACTCACTATAGGGGGAATTGTGAGCGGATAACAATTT |
| | CCCTCTAGAAATAATTTTGTTTAAACTTTAAGAAGGAGATATACATATGCACCATCATCATC |
| | ATCATTCTGGATCCATGGGGAAGCAAGAAGATGCAGAGCTCGTCATCATACCTTTCCCTTT |
| | CTCCGGACACATTCTCGCAACAATCGAACTCGCCAAACGTCTCATAAGTCAAGACAATCCT |
| | CGGATCCACACCATCACCATCCTCTATTGGGGATTACCTTTTATTCCTCAAGCTGACACAAT |
| | CGCTTTCCTCCGATCCCTAGTCAAAAATGAGCCTCGTATCCGTCTCGTTACGTTGCCCGAAG |
| | TCCAAGACCCTCCACCAATGGAACTCTTTGTGGAATTTGCCGAATCTTACATTCTTGAATAC |
| | GTCAAGAAAATGGTTCCCATCATCAGAGAAGCTCTCTCCACTCTCTTGTCTTCCCGCGATGA |
| | ATCGGGTTCAGTTCGTGTGGCTGGATTGGTTCTTGACTTCTTCTGCGTCCCTATGATCGATG |
| | TAGGAAACGAGTTTAATCTCCCTTCTTACATTTTCTTGACGTGTAGCGCAGGGTTCTTGGGT |
| | ATGATGAAGTATCTTCCAGAGAGACACCGCGAAATCAAATCGGAATTCAACCGGAGCTTC |
| | AACGAGGAGTTGAATCTCATTCCTGGTTATGTCAACTCTGTTCCTACTAAGGTTTTGCCGTC |
| | AGGTCTATTCATGAAAGAGACCTACGAGCCTTGGGTCGAACTAGCAGAGAGGTTTCCTGA |
| | AGCTAAGGGTATTTTGGTTAATTCATACACAGCTCTCGAGCCAAACGGTTTTAAATATTTCG |
| | ATCGTTGTCCGGATAACTACCCAACCATTTACCCAATCGGGCCCATTTTGAACCTTGAAAAC |
| | AAAAAAGACGATGCTAAAACCGACGAGATTATGAGGTGGTTAAATGAGCAACCGGAAAG |
| | CTCGGTTGTGTTTTTATGTTTCGGAAGCATGGGTAGCTTTAACGAGAAACAAGTGAAGGA |
| | GATTGCGGTTGCGATTGAAAGAAGTGGACATAGATTTTTATGGTCGCTTCGTCGTCCGACA |
| | CCGAAAGAAAAGATAGAGTTTCCGAAAGAATATGAAAACTTGGAAGAAGTTCTTCCAGAG |
| | GGATTCCTTAAACGTACATCAAGCATCGGGAAGGTGATCGGGTGGGCCCCACAAATGGCG |
| | GTGTTGTCTCACCCGTCAGTTGGTGGGTTTGTGTCGCATTGTGGTTGGAACTCGACATTGG |
| | AGAGTATGTGGTGTGGGGTTCCGATGGCAGCTTGGCCATTATATGCTGAACAAACGTTGA |
| | ATGCTTTTCTACTTGTGGTGGAACTGGGATTGGCGGCGGAGATTAGGATGGATTATCGGA |
| | CGGATACGAAAGCGGGGTATGACGGTGGGATGGAGGTGACGGTGGAGGAGATTGAAGA |
| | TGGAATTAGGAAGTTGATGAGTGATGGTGAGATTAGAAATAAGGTGAAAGATGTGAAAG |
| | AGAAGAGTAGAGCTGCGGTTGTTGAAGGTGGATCTTCTTACGCATCCATTGGAAAATTCAT |
| | CGAGCATGTATCGAATGTTACGATTTAAGGTCGACAAGCTTGGCGGCCGCGCCACGCGAT |
| | CGCTGACGTCGGTACCCTCGAGTCTGGTAAAGAAACCGCTGCTGCGAAATTTGAACGCCA |
| | GCACATGGACTCGTCTACTAGCGCAGCTTAATTAACCTAGG |

What is claimed is:

1. A recombinant host, comprising an operative engineered biosynthetic pathway comprising one or more heterologous genes, wherein each of the one or more heterologous genes encodes a polypeptide capable of catalyzing formation of a melanin precursor from tyrosine.

2. The recombinant host of claim 1, wherein the melanin precursor is a hydroxyindole.

3. A recombinant host, comprising an operative engineered biosynthetic pathway comprising one or more heterologous genes, wherein each of the one or more heterologous genes encodes a polypeptide capable of catalyzing formation of a dihydroxyindole.

4. A recombinant host, comprising an operative engineered biosynthetic pathway comprising:
one or more heterologous genes wherein each of the one or more heterologous genes encodes a polypeptide capable of catalyzing the formation of a melanin precursor from tyrosine; and
one or more heterologous genes each encoding a glycosyltransferase (UGT) polypeptide,
wherein the melanin precursor is a dihydroxyindole, and
wherein each of the UGT polypeptides is capable of glycosylating the dihydroxyindole.

5. The recombinant host of claim 4, wherein the host is capable of producing a glycosylated dihydroxyindole.

6. The recombinant host of claim 5, wherein the glycosylated dihydroxyindole is mono-glucosylated 5,6-DHI in position 5 (β-D-5Glc-6OH-indole; C1), mono-glucosylated 5,6-DHI in position 6 (C2), or di-glucosylated 5,6-DHI.

7. The recombinant host of claim 5, wherein the host is capable of producing a plurality of glycosylated dihydroxyindoles.

8. A recombinant host, comprising:
(a) a gene encoding a first polypeptide capable of catalyzing the formation of 5,6-dihydroxyindole (DHI); and
(b) a gene encoding a glycosyltransferase (UGT) polypeptide, wherein the UGT polypeptide is capable of glycosylation of 5,6-DHI;
wherein at least one of the genes is a recombinant gene, and
wherein the recombinant host produces a glycosylated 5,6-DHI.

9. The recombinant host of claim 8, wherein
(a) the first polypeptide comprises a tyrosinase polypeptide having at least 50% identity to an amino acid sequence set forth in SEQ ID NO: 2, 4, 6, 8 or 10; and
(b) the UGT polypeptide comprises a UGT polypeptide having at least 50% identity to an amino acid sequence set forth in SEQ ID NO: 12, 14, 16, 18, 20, 22, 24, 26, 28, 30, 32, 34, 36, 38, 40, 42, 44, 46, 48, 50, or 52.

10. A method of producing glycosylated DHI, comprising:
(a) growing the recombinant host of any one of claims 1-9 in a culture medium, wherein a glycosylated DHI is synthesized by the recombinant host; and
(b) optionally isolating the glycosylated DHI.

11. A method for producing glycosylated 5,6-DHI from a bioconversion reaction, comprising:
(a) growing a recombinant host in a culture medium, wherein the host expresses a gene encoding a UGT polypeptide capable of glycosylation of a melanin precursor;
(b) adding a melanin precursor comprising 5,6-DHI to the culture medium to induce glycosylation of the melanin precursor; and
(c) optionally isolating the glycosylated 5,6-DHI.

12. The method of claim 11 further comprising isolating the UGT polypeptide from the recombinant host prior to addition of the melanin precursor.

13. The method of claim 12, wherein the melanin precursor is glycosylated in an in vitro reaction.

14. The method of claim 13, wherein the UGT polypeptide comprises a UGT polypeptide having at least 50% identity to an amino acid sequence set forth in SEQ ID NO: 12, 14, 16, 18, 20, 22, 24, 26, 28, 30, 32, 34, 36, 38, 40, 42, 44, 46, 48, 50, or 52.

15. The recombinant host of any one of claims 1-9, wherein the recombinant host comprises a yeast cell, a plant cell, a mammalian cell, an insect cell, a fungal cell, or a bacterial cell.

16. The recombinant host of claim 15, wherein the recombinant host is a bacterial cell that is an Escherichia cell, a Lactobacillus cell, a Lactococcus cell, a Cornebacterium cell, an Acetobacter cell, an Acinetobacter cell, or a Pseudomonas cell.

17. The recombinant host of claim 15, wherein the recombinant host is a yeast cell that is from a Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

18. The recombinant host of claim 17, wherein the yeast cell is a cell from the Saccharomyces cerevisiae species.

19. A method for producing glycosylated 5,6-DHI from an in vitro reaction comprising contacting 5,6-DHI with one or more UGT polypeptides in the presence of one or more UDP-sugars.

20. The method of claim 19, wherein the UGT polypeptide comprises a UGT polypeptide having at least 50% identity to an amino acid sequence set forth in SEQ ID NO: 12, 14, 16, 18, 20, 22, 24, 26, 28, 30, 32, 34, 36, 38, 40, 42, 44, 46, 48, 50, or 52.

21. The method of claim 19 or 20, wherein the one or more UDP-sugars comprises plant-derived or synthetic glucose.

22. A recombinant host, comprising an operative engineered biosynthetic pathway comprising a heterologous gene encoding a tyrosinase polypeptide, wherein the tyrosinase polypeptide is capable of catalyzing formation of a melanin precursor from tyrosine.

23. The recombinant host of claim 22, wherein the melanin precursor is a hydroxyindole.

24. A recombinant host, comprising an operative engineered biosynthetic pathway comprising a heterologous gene encoding a tyrosinase polypeptide, wherein the tyrosinase polypeptide is capable of catalyzing formation of a dihydroxyindole.


SEQUENCE LISTING (53 sequences)

SEQ ID NO: 1 - DNA, 1620 bp, Artificial Sequence
  Codon optimized for yeast
atggcctctgtcgaacctattaagaccttcgaaattagacaaaagggtccagttgaaactaaggccgaaagaaagtctatcagagacttgaacgaagaagaattggacaagttgattgaagcctggagatggattcaagatccagctagaactggtgaagattcctttttttacttggccggtttacatggtgaaccttttagaggtgctggttacaacaattctcattggtggggtggttattgtcatcatggtaacattttgttcccaacctggcatagagcttatttgatggctgttgaaaaggctttgagaaaagcctgtccagatgtttctttgccatattgggatgaatctgatgacgaaactgctaagaaaggtatcccattgatcttcacccaaaaagaatacaagggtaagccaaacccattatactcttacaccttctccgaaagaatcgttgatagattggctaagtttccagatgccgattactctaaaccacaaggttacaagacttgcagatatccatattctggtttgtgcggtcaagatgatattgctattgctcaacaacacaacaatttcttggacgccaatttcaatcaagaacaaatcaccggtttgttgaactccaatgttacttcttggttgaacttgggtcaattcaccgatattgaaggtaagcaagttaaggctgataccagatggaagattagacaatgtttgttgaccgaagaatacaccgttttctctaacactacttctgctcaaagatggaacgatgaacaattccatccattggaatctggtggtaaagaaactgaagctaaggctacttctttggctgttccattagaatctccacataacgatatgcatttggccattggtggtgttcaaattccaggttttaacgttgatcaatacgctggtgctaatggtgatatgggtgaaaatgatactgcttccttcgatccaatcttctactttcatcattgcttcatcgactacttgttctggacttggcaaaccatgcataagaaaactgatgcctcccaaattaccatcttgccagaatatccaggtacaaactctgttgattctcaaggtccaactccaggtatttctggtaatacttggttgactttggataccccattggatccattcagagaaaatggtgacaaagtcacctctaacaagttgttgaccttgaaggatttgccatacacttacaaagctccaacttctggtactggttctgtttttaatgatgtcccaagattgaactacccattgtctccaccaattttgagagtttccggtattaacagagcttccattgctggttcttttgccttggctatttcacaaactgatcatactggtaaggctcaagtcaagggtattgaatctgttttgtctagatggcatgttcaaggttgtgctaactgtcaaactcatttgtctactactgctttcgtccctttgttcgaattgaatgaagatgacgccaagagaaagcacgctaacaatgaattagctgttcacttgcataccagaggtaatccaggtggtcaaagagttagaaacgttactgttggtactatgagataa

SEQ ID NO: 2 - protein, 539 aa, Artificial Sequence
  Codon optimized for yeast
MASVEPIKTFEIRQKGPVETKAERKSIRDLNEEELDKLIEAWRWIQDPARTGEDSFFYLAGLHGEPFRGAGYNNSHWWGGYCHHGNILFPTWHRAYLMAVEKALRKACPDVSLPYWDESDDETAKKGIPLIFTQKEYKGKPNPLYSYTFSERIVDRLAKFPDADYSKPQGYKTCRYPYSGLCGQDDIAIAQQHNNFLDANFNQEQITGLLNSNVTSWLNLGQFTDIEGKQVKADTRWKIRQCLLTEEYTVFSNTTSAQRWNDEQFHPLESGGKETEAKATSLAVPLESPHNDMHLAIGGVQIPGFNVDQYAGANGDMGENDTASFDPIFYFHHCFIDYLFWTWQTMHKKTDASQITILPEYPGTNSVDSQGPTPGISGNTWLTLDTPLDPFRENGDKVTSNKLLTLKDLPYTYKAPTSGTGSVFNDVPRLNYPLSPPILRVSGINRASIAGSFALAISQTDHTGKAQVKGIESVLSRWHVQGCANCQTHLSTTAFVPLFELNEDDAKRKHANNELAVHLHTRGNPGGQRVRNVTVGTMR

SEQ ID NO: 3 - DNA, 1878 bp, Artificial Sequence
  Codon optimized for yeast
atgtccagagttgttatcaccggtgtttctggtactgttgctaatagattggaaatcaacgacttcgtcaagaacgacaagttcttctcattgtacattcaagccttgcaagtcatgtcatctgttccaccacaagaaaacgttagatccttctttcaaatcggtggtattcatggtttgccatatactccatgggatggtattactggtgatcaaccatttgatccaaatactcaatggggtggttactgtactcatggttctgttttgtttccaacttggcatagaccatacgtcttgttgtatgaacaaatcttgcacaagcacgttcaagatattgctgctacttataccacttctgataaggctgcttgggttcaagctgctgctaatttgagacaaccatattgggattgggctgctaatgctgttcctccagatcaagttattgcttctaagaaggttaccatcactggttctaatggtcacaaggttgaagttgacaacccattataccattacaagttccacccaatcgattcctcatttccaagaccatattctgaatggccaactaccttaagacaacctaattcttctagaccaaacgccactgataatgtcgctaagttgagaaatgttttgagagcttcccaagaaaacatcacctctaacacttactctatgttgaccagagttcatacttggaaggctttctctaatcatactgttggtgatggtggttctacctctaattctttggaagctattcatgatggtatccacgttgatgtaggtggtggtggtcatatggctgatccagctgttgctgcttttgatcctattttcttcttgcatcactgcaacgtcgacagattattgtctttgtgggcagctattaacccaggtgtttgggtttctccaggtgattctgaagatggtactttcattttgccacctgaagctccagttgatgtttctactccattaactccattctctaacaccgaaactactttttgggcttctggtggtattacagatacaactaagttgggttacacctacccagaattcaatggtttggatttgggtaatgctcaagctgttaaggctgcaattggtaacatcgttaacagattatacggtgcctctgttttttctggttttgctgctgcaacttctgctattggtgctggttcagttgcttctttggctgctgatgttccattggaaaaagctccagctcctgctccagaagctgccgctcaatctccagttccagcaccagctcatgttgaaccagctgttagagctgtttctgttcatgctgcagctgctcaaccacatgctgaaccaccagttcacgtttctgccggtggtcatccatctccacatggtttttatgattggaccgctagaatcgaattcaagaagtacgaattcggttcctccttttccgttttgttgtttttgggtccagttcctgaagatccagaacaatggttagtttctccaaatttcgttggtgctcatcatgcttttgttaattctgctgctggtcattgtgctaactgtagaaatcaaggtaacgttgttgttgaaggtttcgttcatttgaccaagtacatttctgaacatgccggtttgagatctttgaacccagaagttgttgaaccttacttgaccaacgaattgcattggagagttttgaaagctgatggtagtgttggtcaattggaatccttggaagtttctgtttatggtactccaatgaacttgccagttggtgctatgtttcctgttccaggtaatagaagacatttccatggtatcactcacggtagagttggtggtagtagacatgctatagtttaa

SEQ ID NO: 4 - protein, 625 aa, Artificial Sequence
  Codon optimized for yeast
MSRVVITGVSGTVANRLEINDFVKNDKFFSLYIQALQVMSSVPPQENVRSFFQIGGIHGLPYTPWDGITGDQPFDPNTQWGGYCTHGSVLFPTWHRPYVLLYEQILHKHVQDIAATYTTSDKAAWVQAAANLRQPYWDWAANAVPPDQVIASKKVTITGSNGHKVEVDNPLYHYKFHPIDSSFPRPYSEWPTTLRQPNSSRPNATDNVAKLRNVLRASQENITSNTYSMLTRVHTWKAFSNHTVGDGGSTSNSLEAIHDGIHVDVGGGGHMADPAVAAFDPIFFLHHCNVDRLLSLWAAINPGVWVSPGDSEDGTFILPPEAPVDVSTPLTPFSNTETTFWASGGITDTTKLGYTYPEFNGLDLGNAQAVKAAIGNIVNRLYGASVFSGFAAATSAIGAGSVASLAADVPLEKAPAPAPEAAAQSPVPAPAHVEPAVRAVSVHAAAAQPHAEPPVHVSAGGHPSPHGFYDWTARIEFKKYEFGSSFSVLLFLGPVPEDPEQWLVSPNFVGAHHAFVNSAAGHCANCRNQGNVVVEGFVHLTKYISEHAGLRSLNPEVVEPYLTNELHWRVLKADGSVGQLESLEVSVYGTPMNLPVGAMFPVPGNRRHFHGITHGRVGGSRHAIV

SEQ ID NO: 5 - DNA, 1857 bp, Artificial Sequence
  Codon optimized for yeast
atgtcccacttcatcgttactggtccagttggtggtcaaactgaaggtgctccagctccaaatagattggaaatcaacgatttcgtcaagaacgaagaatttttctcattatacgttcaagccttggacatcatgtacggtttgaaacaagaagaattgatctccttcttccaaatcggtggtattcatggtttgccatatgttgcttggtctgatgctggtgctgatgatccagctgaaccatctggttactgtactcatggttctgttttgtttccaacttggcatagaccatacgttgccttgtatgaacaaatcttgcataagtacgctggtgaaattgctgataagtacactgttgataagccaagatggcaaaaagctgctgctgatttgagacaaccattttgggattgggctaagaatactttgccaccaccagaagttatttctttggataaggttactatcaccaccccagatggtcaaagaactcaagttgataatccattgagaagatacagattccacccaatcgatccatcttttccagaaccatattctaattggccagctactttgagacatccaacatctgatggttctgatgctaaggataacgttaaggatttgactactaccttgaaggctgatcaaccagatattactactaagacctacaacttgttgaccagagttcatacttggccagccttttctaatcatactccaggtgatggtggttcctcttctaattctttggaagccattcatgatcacatccacgattctgtaggtggtggtggtcaaatgggtgatccatctgttgctggttttgatccaattttcttcttgcatcattgccaagtcgatagattattggctttgtggtctgctttgaatccaggtgtttgggttaattcctcatcatctgaagatggtacttacaccattccaccagattctactgttgatcaaactactgctttaaccccattctgggatactcaatctactttctggacctcttttcaatctgctggtgtttctccatctcaattcggttattcttacccagaattcaatggtttgaacttgcaagaccaaaaggctgttaaggatcatattgccgaagtcgtcaatgaattatacggtcacagaatgagaaagacctttccatttccacaattgcaagctgtttctgttgctaaacaaggtgatgctgttactccatcagttgctactgattctgtttcttcatctactaccccagctgaaaatccagcttctagagaagatgcttctgataaggatactgaacctacattgaacgttgaagttgctgctccaggtgctcatttgacttctactaagtactgggattggaccgctagaattcacgttaagaaatatgaagtcggtggttctttctccgtcttgttgtttttgggtgctattccagaaaatcctgcagattggagaacatctccaaattatgtcggtggtcatcatgctttcgttaactcttcaccacaaagatgtgctaactgtagaggtcaaggtgatttggttattgaaggtttcgtccatttgaacgaagctattgctagacatgcacacttggattcttttgacccaactgttgttagaccttacttgactagagaattgcattggggtgttatgaaggttaacggtactgttgttccattgcaagatgttccatcattggaagttgttgtcttgtctactccattgactttaccaccaggtgaaccatttccagttccaggtactccagttaaccatcatgatattacacatggtagaccaggtggttctcatcatacacattaa

SEQ ID NO: 6 - protein, 618 aa, Artificial Sequence
  Codon optimized for yeast
MSHFIVTGPVGGQTEGAPAPNRLEINDFVKNEEFFSLYVQALDIMYGLKQEELISFFQIGGIHGLPYVAWSDAGADDPAEPSGYCTHGSVLFPTWHRPYVALYEQILHKYAGEIADKYTVDKPRWQKAAADLRQPFWDWAKNTLPPPEVISLDKVTITTPDGQRTQVDNPLRRYRFHPIDPSFPEPYSNWPATLRHPTSDGSDAKDNVKDLTTTLKADQPDITTKTYNLLTRVHTWPAFSNHTPGDGGSSSNSLEAIHDHIHDSVGGGGQMGDPSVAGFDPIFFLHHCQVDRLLALWSALNPGVWVNSSSSEDGTYTIPPDSTVDQTTALTPFWDTQSTFWTSFQSAGVSPSQFGYSYPEFNGLNLQDQKAVKDHIAEVVNELYGHRMRKTFPFPQLQAVSVAKQGDAVTPSVATDSVSSSTTPAENPASREDASDKDTEPTLNVEVAAPGAHLTSTKYWDWTARIHVKKYEVGGSFSVLLFLGAIPENPADWRTSPNYVGGHHAFVNSSPQRCANCRGQGDLVIEGFVHLNEAIARHAHLDSFDPTVVRPYLTRELHWGVMKVNGTVVPLQDVPSLEVVVLSTPLTLPPGEPFPVPGTPVNHHDITHGRPGGSHHTH

SEQ ID NO: 7 - DNA, 1857 bp, Artificial Sequence
  Codon optimized for yeast
atgtcccactacttggttactggtgctactggtggttctacttctggtgctgctgctccaaatagattggaaatcaacgatttcgtcaagcaagaagatcaattctccttgtacattcaagccttgcaatatatctactcctccaagtcccaagatgacatcgattcttttttccaaatcggtggtattcacggtttgccatatgttccatgggatggtgctggtaacaaaccagttgatactgatgcttgggaaggttactgtactcatggttctgttttgttcccaactttccatagaccatacgtcttgttgattgaacaagctattcaagctgctgctgttgatattgctgctacttatatcgttgatagagccagatatcaagatgctgccttgaatttgagacaaccatattgggattgggctagaaatccagttccaccacctgaagttatttctttggatgaagttaccatcgtcaacccatctggtgaaaagatttctgttccaaacccattgagaagatacaccttccatccaattgatccatcttttccagaaccataccaatcttggtctactactttaagacacccattgtctgatgatgctaacgcttctgataatgtcccagaattgaaagctactttgagatctgctggtccacaattgaaaactaagacctacaacttgttgaccagagttcatacttggccagctttttctaatcatactccagatgatggtggttccacctctaattctttggaaggtattcatgattccgttcacgttgatgttggtggtaatggtcaaatgtctgatccatcagttgctggttttgatccaatcttctttatgcatcatgcccaagtcgacagattattgtctttgtggtctgctttgaatccaagagtttggattactgatggtccttctggtgatggtacttggactattccaccagatactgttgttggtaaagatactgatttgaccccattctggaacacccaatcttcatattggatttctgctaacgttaccgacacttctaaaatgggttatacctacccagaattcaacaacttggatatgggtaacgaagttgctgttagatctgctattgctgcacaagttaacaagttatatggtggtccattcactaagttcgctgctgctatacaacaaccatcttcacaaactactgctgatgcttctactattggtaatgttacttccgatgcctcctctcatttggttgattctaagattaacccaaccccaaacagatctattgatgatgcacctcaagttaagattgcctctaccttgagaaacaacgaacaaaaagaattttgggaatggaccgctagagttcaagtcaaaaagtacgaaattggtggtagtttcaaggtcttgttcttcttgggttcagttccatctgatccaaaagaatgggctactgatccacattttgttggtgcttttcatggtttcgttaactcctctgctgaaagatgtgctaactgtagaagacaacaagatgttgtcttggaaggtttcgtccatttgaatgaaggtattgccaacatctccaacttgaattctttcgatccaatcgttgtcgaaccatacttgaaagaaaacttgcattggagagttcaaaaggtcagtggtgaagttgttaatttggatgctgctacctcattggaagttgttgttgtagctaccagattggaattgccaccaggtgaaatttttccagttcctgctgaaacacatcatcatcaccatattacacatggtagaccaggtggttcaagacattctgttgcttcatcttcatcctaa

SEQ ID NO: 8 - protein, 618 aa, Artificial Sequence
  Codon optimized
MSHYLVTGATGGSTSGAAAPNRLEINDFVKQEDQFSLYIQALQYIYSSKSQDDIDSFFQIGGIHGLPYVPWDGAGNKPVDTDAWEGYCTHGSVLFPTFHRPYVLLIEQAIQAAAVDIAATYIVDRARYQDAALNLRQPYWDWARNPVPPPEVISLDEVTIVNPSGEKISVPNPLRRYTFHPIDPSFPEPYQSWSTTLRHPLSDDANASDNVPELKATLRSAGPQLKTKTYNLLTRVHTWPAFSNHTPDDGGSTSNSLEGIHDSVHVDVGGNGQMSDPSVAGFDPIFFMHHAQVDRLLSLWSALNPRVWITDGPSGDGTWTIPPDTVVGKDTDLTPFWNTQSSYWISANVTDTSKMGYTYPEFNNLDMGNEVAVRSAIAAQVNKLYGGPFTKFAAAIQQPSSQTTADASTIGNVTSDASSHLVDSKINPTPNRSIDDAPQVKIASTLRNNEQKEFWEWTARVQVKKYEIGGSFKVLFFLGSVPSDPKEWATDPHFVGAFHGFVNSSAERCANCRRQQDVVLEGFVHLNEGIANISNLNSFDPIVVEPYLKENLHWRVQKVSGEVVNLDAATSLEVVVVATRLELPPGEIFPVPAETHHHHHITHGRPGGSRHSVASSSS

SEQ ID NO: 9 - DNA, 1878 bp, Artificial Sequence
  Codon optimized
atgtccagagttgttatcaccggtgtttctggtactattgctaacagattggaaatcaacgacttcgtcaagaacgacaagttcttctcattgtacattcaagccttgcaagtcatgtcatctgttccaccacaagaaaacgttagatccttctttcaaatcggtggtattcatggtttgccatatactccatgggatggtattactggtgatcaaccatttgatccaaatactcaatggggtggttactgtactcatggttctgttttgtttccaacttggcatagaccatacgtcttgttgtatgaacaaatcttgcacaagcacgttcaagatattgctgctacttataccacttctgataaggctgcttgggttcaagctgctgctaatttgagacaaccatattgggattgggctgctaatgctgttcctccagatcaagttatcgtttctaagaaggttaccatcactggttctaacggtcataaggttgaagttgacaacccattataccattacaagttccacccaatcgattcctcatttccaagaccatattctgaatggccaactaccttaagacaacctaattcttctagaccaaacgccactgataatgtcgctaagttgagaaatgttttgagagcttcccaagaaaacatcacctctaacacttactctatgttgaccagagttcatacttggaaggctttctctaatcatactgttggtgatggtggttctacctctaattctttggaagctattcatgatggtatccacgttgatgtaggtggtggtggtcatatgggtgatccagctgttgctgcttttgatcctattttcttcttgcatcactgcaacgtcgacagattattgtctttgtgggcagctattaacccaggtgtttgggtttctccaggtgattctgaagatggtactttcattttgccacctgaagctccagttgatgtttctactccattaactccattctctaacaccgaaactactttttgggcttctggtggtattacagatacaactaagttgggttacacctacccagaattcaatggtttggatttgggtaatgctcaagctgttaaggctgcaattggtaacatcgttaacagattatacggtgcctctgttttttctggttttgctgctgcaacttctgctattggtgctggttcagttgcttctttggctgctgatgttccattggaaaaagctccagctcctgctccagaagctgccgctcaaccaccagttccagctccagcacatgttgaaccagctgttagagctgtttctgttcatgctgcagctgctcaacctcatgcagaaccacctgttcatgtttctgccggtggtcatccatctccacatggtttttatgattggaccgctagaatcgaattcaagaagtacgaattcggttcctccttttccgttttgttgtttttgggtccagttcctgaagatccagaacaatggttagtttctccaaatttcgttggtgctcatcatgcttttgttaattctgctgctggtcattgtgctaactgtagatctcaaggtaacgttgttgttgaaggtttcgttcatttgaccaagtacatttctgaacatgccggtttgagatctttgaacccagaagttgttgaaccttacttgaccaacgaattgcattggagagttttgaaagctgatggtagtgttggtcaattggaatccttggaagtttctgtttatggtactccaatgaacttgccagttggtgctatgtttcctgttccaggtaatagaagacatttccatggtatcactcacggtagagttggtggttcaagacatgctatagtttaa

SEQ ID NO: 10 - protein, 625 aa, Artificial Sequence
  Codon optimized
MSRVVITGVSGTIANRLEINDFVKNDKFFSLYIQALQVMSSVPPQENVRSFFQIGGIHGLPYTPWDGITGDQPFDPNTQWGGYCTHGSVLFPTWHRPYVLLYEQILHKHVQDIAATYTTSDKAAWVQAAANLRQPYWDWAANAVPPDQVIVSKKVTITGSNGHKVEVDNPLYHYKFHPIDSSFPRPYSEWPTTLRQPNSSRPNATDNVAKLRNVLRASQENITSNTYSMLTRVHTWKAFSNHTVGDGGSTSNSLEAIHDGIHVDVGGGGHMGDPAVAAFDPIFFLHHCNVDRLLSLWAAINPGVWVSPGDSEDGTFILPPEAPVDVSTPLTPFSNTETTFWASGGITDTTKLGYTYPEFNGLDLGNAQAVKAAIGNIVNRLYGASVFSGFAAATSAIGAGSVASLAADVPLEKAPAPAPEAAAQPPVPAPAHVEPAVRAVSVHAAAAQPHAEPPVHVSAGGHPSPHGFYDWTARIEFKKYEFGSSFSVLLFLGPVPEDPEQWLVSPNFVGAHHAFVNSAAGHCANCRSQGNVVVEGFVHLTKYISEHAGLRSLNPEVVEPYLTNELHWRVLKADGSVGQLESLEVSVYGTPMNLPVGAMFPVPGNRRHFHGITHGRVGGSRHAIV

SEQ ID NO: 11 - DNA, 1446 bp, Arabidopsis thaliana
atggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcgggccgatattatgctccaacgaccgtccgaatttggactcatcggaacgagatcggatcataacttggctagatgaccaacccgagtcatcggtcgtgttcctctgtttcgggagcttgaagaatctcagcgctactcagatcaacgagatagctcaagccttagagatcgttgactgcaaattcatctggtcgtttcgaaccaacccgaaggagtacgcgagcccttacgaggctctaccacacgggttcatggaccgggtcatggatcaaggcattgtttgtggttgggctcctcaagttgaaatcctagcccataaagctgtgggaggattcgtatctcattgtggttggaactcgatattggagagtttgggtttcggcgttccaatcgccacgtggccgatgtacgcggaacaacaactaaacgcgttcacgatggtgaaggagcttggtttagccttggagatgcggttggattacgtgtcggaagatggagatatagtgaaagctgatgagatcgcaggaaccgttagatctttaatggacggtgtggatgtgccgaagagtaaagtgaaggagattgctgaggcgggaaaagaagctgtggacggtggatcttcgtttcttgcggttaaaagattcatcggtgacttgatcgacggcgtttctataagtaagtag

SEQ ID NO: 12 - protein, 481 aa, Arabidopsis thaliana
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILCSNDRPNLDSSERDRIITWLDDQPESSVVFLCFGSLKNLSATQINEIAQALEIVDCKFIWSFRTNPKEYASPYEALPHGFMDRVMDQGIVCGWAPQVEILAHKAVGGFVSHCGWNSILESLGFGVPIATWPMYAEQQLNAFTMVKELGLALEMRLDYVSEDGDIVKADEIAGTVRSLMDGVDVPKSKVKEIAEAGKEAVDGGSSFLAVKRFIGDLIDGVSISK

SEQ ID NO: 13 - DNA, 1425 bp, Arabidopsis thaliana
atggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcccgggtttgttaactccgttccggttaaagttttgccaccgggtttgttcacgactgagtcttacgaagcttgggtcgaaatggcggaaaggttccctgaagccaagggtattttggtcaattcatttgaatctctagaacgtaacgcttttgattatttcgatcgtcgtccggataattacccacccgtttacccaatcgggccaattctatgctccaacgatcgtccgaatttggatttatcggaacgagaccggatcttgaaatggctcgatgaccaacccgagtcatctgttgtgtttctctgcttcgggagcttgaagagtctcgctgcgtctcagattaaagagatcgctcaagccttagagctcgtcggaatcagattcctctggtcgattcgaacggacccgaaggagtacgcgagcccgaacgagattttaccggacgggtttatgaaccgagtcatgggtttgggccttgtttgtggttgggctcctcaagttgaaattctggcccataaagcaattggagggttcgtgtcacactgcggttggaactcgatattggagagtttgcgtttcggagttccaattgccacgtggccaatgtacgcggaacaacaactaaacgcgttcacgattgtgaaggagcttggtttggcgttggagatgcggttggattacgtgtcggaatatggagaaatcgtgaaagctgatgaaatcgcaggagccgtacgatctttgatggacggtgaggatgtgccgaggaggaaactgaaggagattgcggaggcgggaaaagaggctgtgatggacggtggatcttcgtttgttgcggttaaaagattcatagatgggctttga

SEQ ID NO: 14 - protein, 474 aa, Arabidopsis thaliana
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGFVNSVPVKVLPPGLFTTESYEAWVEMAERFPEAKGILVNSFESLERNAFDYFDRRPDNYPPVYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 15 - DNA, 1425 bp, Arabidopsis thaliana
atggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcgggcccattctatgctccaacgatcgtccgaatttggatttatcggaacgagaccggatcttgaaatggctcgatgaccaacccgagtcatctgttgtgtttctctgcttcgggagcttgaagagtctcgctgcgtctcagattaaagagatcgctcaagccttagagctcgtcggaatcagattcctctggtcgattcgaacggacccgaaggagtacgcgagcccgaacgagattttaccggacgggtttatgaaccgagtcatgggtttgggccttgtttgtggttgggctcctcaagttgaaattctggcccataaagcaattggagggttcgtgtcacactgcggttggaactcgatattggagagtttgcgtttcggagttccaattgccacgtggccaatgtacgcggaacaacaactaaacgcgttcacgattgtgaaggagcttggtttggcgttggagatgcggttggattacgtgtcggaatatggagaaatcgtgaaagctgatgaaatcgcaggagccgtacgatctttgatggacggtgaggatgtgccgaggaggaaactgaaggagattgcggaggcgggaaaagaggctgtgatggacggtggatcttcgtttgttgcggttaaaagattcatagatgggctttga

SEQ ID NO: 16 - protein, 474 aa, Arabidopsis thaliana
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 17 - DNA, 1473 bp, Arabidopsis thaliana
atggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcgggcccattttgaaccttgaaaacaaaaaagacgatgctaaaaccgacgagattatgaggtggttaaatgagcaaccggaaagctcggttgtgtttttatgtttcggaagcatgggtagctttaacgagaaacaagtgaaggagattgcggttgcgattgaaagaagtggacatagatttttatggtcgcttcgtcgtccgacaccgaaagaaaagatagagtttccgaaagaatatgaaaacttggaagaagttcttccagagggattccttaaacgtacatcaagcatcgggaaggtgatcgggtgggccccacaaatggcggtgttgtctcacccgtcagttggtgggtttgtgtcgcattgtggttggaactcgacattggagagtatgtggtgtggggttccgatggcagcttggccattatatgctgaacaaacgttgaatgcttttctacttgtggtggaactgggattggcggcggagattaggatggattatcggacggatacgaaagcggggtatgacggtgggatggaggtgacggtggaggagattgaagatggaattaggaagttgatgagtgatggtgagattagaaataaggtgaaagatgtgaaagagaagagtagagctgcggttgttgaaggtggatcttcttacgcatccattggaaaattcatcgagcatgtatcgaatgttacgatttaa

SEQ ID NO: 18 - protein, 490 aa, Arabidopsis thaliana
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 19 - DNA, 1473 bp, Arabidopsis thaliana
atggcgaagcagcaagaagcagagctcatcttcatcccatttccaatccccggacacattctcgccacaatcgaactcgcgaaacgtctcatcagtcaccaacctagtcggatccacaccatcaccatcctccattggagcttaccttttcttcctcaatctgacactatcgccttcctcaaatccctaatcgaaacagagtctcgtatccgtctcattaccttacccgatgtccaaaaccctccaccaatggagctatttgtgaaagcttccgaatcttacattcttgaatacgtcaagaaaatggttcctttggtcagaaacgctctctccactctcttgtcttctcgtgatgaatcggattcagttcatgtcgccggattagttcttgatttcttctgtgtccctttgatcgatgtcggaaacgagtttaatctcccttcttacatcttcttgacgtgtagcgcaagtttcttgggtatgatgaagtatcttctggagagaaaccgcgaaaccaaaccggaacttaaccggagctctgacgaggaaacaatatcagttcctggttttgttaactccgttccggttaaagttttgccaccgggtttgttcacgactgagtcttacgaagcttgggtcgaaatggcggaaaggttccctgaagccaagggtattttggtcaattcatttgaatctctagaacgtaacgcttttgattatttcgatcgtcgtccggataattacccacccgtttacccaatcgggcccattttgaaccttgaaaacaaaaaagacgatgctaaaaccgacgagattatgaggtggttaaatgagcaaccggaaagctcggttgtgtttttatgtttcggaagcatgggtagctttaacgagaaacaagtgaaggagattgcggttgcgattgaaagaagtggacatagatttttatggtcgcttcgtcgtccgacaccgaaagaaaagatagagtttccgaaagaatatgaaaacttggaagaagttcttccagagggattccttaaacgtacatcaagcatcgggaaggtgatcgggtgggccccacaaatggcggtgttgtctcacccgtcagttggtgggtttgtgtcgcattgtggttggaactcgacattggagagtatgtggtgtggggttccgatggcagcttggccattatatgctgaacaaacgttgaatgcttttctacttgtggtggaactgggattggcggcggagattaggatggattatcggacggatacgaaagcggggtatgacggtgggatggaggtgacggtggaggagattgaagatggaattaggaagttgatgagtgatggtgagattagaaataaggtgaaagatgtgaaagagaagagtagagctgcggttgttgaaggtggatcttcttacgcatccattggaaaattcatcgagcatgtatcgaatgttacgatttaa

SEQ ID NO: 20 - protein, 490 aa, Arabidopsis thaliana
MAKQQEAELIFIPFPIPGHILATIELAKRLISHQPSRIHTITILHWSLPFLPQSDTIAFLKSLIETESRIRLITLPDVQNPPPMELFVKASESYILEYVKKMVPLVRNALSTLLSSRDESDSVHVAGLVLDFFCVPLIDVGNEFNLPSYIFLTCSASFLGMMKYLLERNRETKPELNRSSDEETISVPGFVNSVPVKVLPPGLFTTESYEAWVEMAERFPEAKGILVNSFESLERNAFDYFDRRPDNYPPVYPIGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 21 - DNA, 1443 bp, Arabidopsis thaliana
atgaagacagcagagctcatattcgttcctctgccggagaccggccatctcttgtcaacgatcgagtttggaaagcgtctactcaatctagaccgtcggatttctatgattacaatcctctccatgaatcttccttacgctcctcacgccgacgcttctcttgcttcgctaacagcctccgagcctggtatccgaatcatcagtctcccggagatccacgatccacctccgatcaagcttcttgacacttcctccgagacttacatcctcgatttcatccataaaaacataccttgtctcagaaaaaccatccaagatttagtctcatcatcatcatcttccggaggtggtagtagtcatgtcgccggcttgattcttgatttcttctgcgttggtttgatcgacatcggccgtgaggtaaaccttccttcctatatcttcatgacttccaactttggtttcttaggggttctacagtatctcccggaacgacaacgtttgactccgtcggagttcgatgagagctccggcgaggaagagttacatattccggcgtttgtgaaccgtgttcccgccaaggttctgccgccaggtgtgttcgataaactctcttacgggtctctggtcaaaatcggcgagcgattacatgaagccaagggtattttggttaattcatttacccaagtggagccttatgctgctgaacatttttctcaaggacgagattaccctcacgtgtatcctgttgggccggttctcaacttaacgggccgtacaaatccgggtctagcttcggcccaatataaagagatgatgaagtggcttgacgagcaaccagactcgtcggttttgttcctgtgtttcgggagcatgggagtcttccctgcacctcagatcacagagattgctcacgcgctcgagcttatcgggtgcaggttcatctgggcgatccgtacgaacatggcgggagatggcgatcctcaggagccgcttccagaaggatttgtcgatcgaacaatgggccgtggaattgtgtgtagttgggctccacaagtggatatcttggcccacaaggcaacaggtggattcgtttctcactgcgggtggaattccgtccaagagagtctatggtacggtgtacctattgcaacgtggccaatgtatgcggagcaacaactgaacgcatttgagatggtgaaggagttgggcttagcagtggagataaggcttgactacgtggcggatggtgatagggttactttggagatcgtgtcagccgatgaaatagccacagccgtccgatcattgatggatagtgataaccccgtgagaaagaaggttatagaaaaatcttcagtggcgaggaaagctgttggtgatggtgggtcttctacggtggccacatgtaattttatcaaagatattcttggggatcacttttga

SEQ ID NO: 22 - protein, 480 aa, Arabidopsis thaliana
MKTAELIFVPLPETGHLLSTIEFGKRLLNLDRRISMITILSMNLPYAPHADASLASLTASEPGIRIISLPEIHDPPPIKLLDTSSETYILDFIHKNIPCLRKTIQDLVSSSSSSGGGSSHVAGLILDFFCVGLIDIGREVNLPSYIFMTSNFGFLGVLQYLPERQRLTPSEFDESSGEEELHIPAFVNRVPAKVLPPGVFDKLSYGSLVKIGERLHEAKGILVNSFTQVEPYAAEHFSQGRDYPHVYPVGPVLNLTGRTNPGLASAQYKEMMKWLDEQPDSSVLFLCFGSMGVFPAPQITEIAHALELIGCRFIWAIRTNMAGDGDPQEPLPEGFVDRTMGRGIVCSWAPQVDILAHKATGGFVSHCGWNSVQESLWYGVPIATWPMYAEQQLNAFEMVKELGLAVEIRLDYVADGDRVTLEIVSADEIATAVRSLMDSDNPVRKKVIEKSSVARKAVGDGGSSTVATCNFIKDILGDHF

SEQ ID NO: 23 - DNA, 1425 bp, Arabidopsis thaliana
atgtccacctcagagcttgttttcatcccatctcccggagctggccatctaccaccaacggtcgagctcgcaaagcttctgttacatcgcgatcaacgactttcggtcacaatcatcgtcatgaatctctggttaggtccaaaacacaacactgaagcacgaccttgtgttcccagtttacggttcgttgacatcccttgcgatgagtccaccatggctctcatctcacccaatacttttatatctgcgttcgttgaacaccacaaaccgcgtgttagagacatagtccgaggtataattgagtctgactcggttcgactcgctgggttcgttcttgatatgttttgtatgccgatgagtgatgttgcaaacgagtttggagttccgagttacaattatttcacatccggtgcagccacgttagggttgatgtttcaccttcaatggaaacgtgatcatgaaggttatgatgcaaccgagttgaaaaactcggatactgagttgtctgttccgagttatgttaacccggttcctgctaaggttttaccggaagtggtgttggataaagaaggtgggtccaaaatgtttcttgaccttgcggaaaggattcgcgagtcgaagggtataatagtaaattcatgtcaggcgattgaaagacacgcgctcgagtacctttcaagcaacaataacggtatcccacctgttttcccggttggtccgattttgaaccttgaaaacaaaaaagacgatgctaaaaccgacgagattatgaggtggttaaatgagcaaccggaaagctcggttgtgtttttatgtttcggaagcatgggtagctttaacgagaaacaagtgaaggagattgcggttgcgattgaaagaagtggacatagatttttatggtcgcttcgtcgtccgacaccgaaagaaaagatagagtttccgaaagaatatgaaaacttggaagaagttcttccagagggattccttaaacgtacatcaagcatcgggaaggtgatcgggtgggccccacaaatggcggtgttgtctcacccgtcagttggtgggtttgtgtcgcattgtggttggaactcgacattggagagtatgtggtgtggggttccgatggcagcttggccattatatgctgaacaaacgttgaatgcttttctacttgtggtggaactgggattggcggcggagattaggatggattatcggacggatacgaaagcggggtatgacggtgggatggaggtgacggtggaggagattgaagatggaattaggaagttgatgagtgatggtgagattagaaataaggtgaaagatgtgaaagagaagagtagagctgcggttgttgaaggtggatcttcttacgcatccattggaaaattcatcgagcatgtatcgaatgttacgatttaa

SEQ ID NO: 24 - protein, 474 aa, Arabidopsis thaliana
MSTSELVFIPSPGAGHLPPTVELAKLLLHRDQRLSVTIIVMNLWLGPKHNTEARPCVPSLRFVDIPCDESTMALISPNTFISAFVEHHKPRVRDIVRGIIESDSVRLAGFVLDMFCMPMSDVANEFGVPSYNYFTSGAATLGLMFHLQWKRDHEGYDATELKNSDTELSVPSYVNPVPAKVLPEVVLDKEGGSKMFLDLAERIRESKGIIVNSCQAIERHALEYLSSNNNGIPPVFPVGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 25 - DNA, 1443 bp, Arabidopsis thaliana
atggaggaatccaaaacacctcacgttgcgatcataccaagtccgggaatgggtcatctcataccactcgtcgagtttgctaaacgactcgtccatcttcacggcctcaccgttaccttcgtcatcgccggcgaaggtccaccatcaaaagctcagagaaccgtcctcgactctctcccttcttcaatctcctccgtctttctccctcctgttgatctcaccgatctctcttcgtccactcgcatcgaatctcggatctccctcaccgtgactcgttcaaacccggagctccggaaagtcttcgactcgttcgtggagggaggtcgtttgccaacggcgctcgtcgtcgatctcttcggtacggacgctttcgacgtggccgtagaatttcacgtgccaccgtatattttctacccaacaacggccaacgtcttgtcgttttttctccatttgcctaaactagacgaaacggtgtcgtgtgagttcagggaattaaccgaaccgcttatgcttcctggatgtgtaccggttgccgggaaagatttccttgacccggcccaagaccggaaagacgatgcatacaaatggcttctccataacaccaagaggtacaaagaagccgaaggtattcttgtgaataccttctttgagctagagccaaatgctataaaggccttgcaagaaccgggtcttgataaaccaccggtttatccggttggaccgttggttaacattggtaagcaagaggctaagcaaaccgaagagtctgaatgtttaaagtggttggataaccagccgctcggttcggttttatatgtgtcctttggtagtggcggtaccctcacatgtgagcagctcaatgagcttgctcttggtcttgcagatagtgagcaacggtttctttgggtcatacgaagtcctagtgggatcgctaattcgtcgtattttgattcacatagccaaacagatccattgacatttttaccaccgggatttttagagcggactaaaaaaagaggttttgtgatccctttttgggctccacaagcccaagtcttggcgcatccatccacgggaggatttttaactcattgtggatggaattcgactctagagagtgtagtaagcggtattccacttatagcatggccattatacgcagaacagaagatgaatgcggttttgttgagtgaagatattcgtgcggcacttaggccgcgtgccggggacgatgggttagttagaagagaagaggtggctagagtggtaaaaggattgatggaaggtgaagaaggcaaaggagtgaggaacaagatgaaggagttgaaggaagcagcttgtagggtgttgaaggatgatgggacttcgacaaaagcacttagtcttgtggccttaaagtggaaagcccacaaaaaagagttagagcaaaatggcaaccactaa

SEQ ID NO: 26 - protein, 480 aa, Arabidopsis thaliana
MEESKTPHVAIIPSPGMGHLIPLVEFAKRLVHLHGLTVTFVIAGEGPPSKAQRTVLDSLPSSISSVFLPPVDLTDLSSSTRIESRISLTVTRSNPELRKVFDSFVEGGRLPTALVVDLFGTDAFDVAVEFHVPPYIFYPTTANVLSFFLHLPKLDETVSCEFRELTEPLMLPGCVPVAGKDFLDPAQDRKDDAYKWLLHNTKRYKEAEGILVNTFFELEPNAIKALQEPGLDKPPVYPVGPLVNIGKQEAKQTEESECLKWLDNQPLGSVLYVSFGSGGTLTCEQLNELALGLADSEQRFLWVIRSPSGIANSSYFDSHSQTDPLTFLPPGFLERTKKRGFVIPFWAPQAQVLAHPSTGGFLTHCGWNSTLESVVSGIPLIAWPLYAEQKMNAVLLSEDIRAALRPRAGDDGLVRREEVARVVKGLMEGEEGKGVRNKMKELKEAACRVLKDDGTSTKALSLVALKWKAHKKELEQNGNH

SEQ ID NO: 27 - DNA, 1443 bp, Arabidopsis thaliana
atggcggaagcaaacactccacacatagcaatcatgccgagtcccggtatgggtcaccttatcccattcgtcgagttagcaaagcgactcgttcagcacgactgtttcaccgtcacaatgatcatctccggtgaaacttcgccgtctaaggcacaaagatccgttctcaactctctcccttcctccatagcctccgtatttctccctcccgccgatctttccgatgttccctccacagcgcgaatcgaaactcgggccatgctcaccatgactcgttccaatccggcgctccgggagctttttggctctttatcaacgaagaaaagtctcccggcggttctcgtcgtcgatatgtttggtgcggatgcgttcgacgtggccgttgacttccacgggtcaccatacattttctatgcatccaatgcaaacgtcttgtcgttttttcttcacttgccgaaactagacaaaacggtgtcgtgtgagtttaggtacttaaccgaaccgcttaagattcccggctgtgtcccgataaccggtaaggactttcttgatacggttcaagaccgaaacgacgacgcatacaaattgcttctccataacaccaagaggtacaaagaagctaaagggattctagtgaattccttcgttgatttagagtcgaatgcaataaaggccttacaagaaccggctcctgataaaccaacggtatacccgattgggccgctggttaacacaagttcatctaatgttaacttggaagacaagttcggatgtttaagttggctagacaaccaaccattcggctcggttctatacatatcatttggaagcggcggaacacttacatgtgagcagtttaatgagcttgctattggtcttgcggagagcggaaaacggtttatttgggtcatacgaagtccaagcgagatagttagttcgtcgtatttcaatccacacagcgagacagaccccttttcgtttttaccaattgggttcttagaccgaaccaaagagaaaggtttggtggttccatcatgggctccacaggttcaaatcctggctcatccatccacatgcgggtttttaacacactgtggatggaattcgaccttagaaagcattgtaaacggtgtaccactcatagcgtggcctttattcgcggagcaaaagatgaatacattgctactcgtggaggatgttggagcggctctaagaatccatgcgggtgaagatgggattgtacggagggaagaagtggtgagagtggtgaaggcactgatggaaggtgaagagggaaaagccataggaaataaagtgaaggagttgaaagaaggagttgttagagtcttgggtgacgatggattgtccagcaagtcatttggtgaagttttgttaaagtggaaaacgcaccagcgagatatcaaccaagagacgtcccactag

SEQ ID NO: 28 - protein, 480 aa, Arabidopsis thaliana
MAEANTPHIAIMPSPGMGHLIPFVELAKRLVQHDCFTVTMIISGETSPSKAQRSVLNSLPSSIASVFLPPADLSDVPSTARIETRAMLTMTRSNPALRELFGSLSTKKSLPAVLVVDMFGADAFDVAVDFHGSPYIFYASNANVLSFFLHLPKLDKTVSCEFRYLTEPLKIPGCVPITGKDFLDTVQDRNDDAYKLLLHNTKRYKEAKGILVNSFVDLESNAIKALQEPAPDKPTVYPIGPLVNTSSSNVNLEDKFGCLSWLDNQPFGSVLYISFGSGGTLTCEQFNELAIGLAESGKRFIWVIRSPSEIVSSSYFNPHSETDPFSFLPIGFLDRTKEKGLVVPSWAPQVQILAHPSTCGFLTHCGWNSTLESIVNGVPLIAWPLFAEQKMNTLLLVEDVGAALRIHAGEDGIVRREEVVRVVKALMEGEEGKAIGNKVKELKEGVVRVLGDDGLSSKSFGEVLLKWKTHQRDINQETSH

SEQ ID NO: 29 - DNA, 1446 bp, Arabidopsis thaliana
atggcagatggaaacactccacatgtagcaatcataccaagtcccggtataggtcacctcatcccactcgtcgagttagcaaagcgactccttgacaatcacggtttcaccgtcactttcatcatccccggcgattctcctccgtctaaggctcaaagatccgttctcaactctctcccttcctccatagcctccgtcttcctccctcccgccgatctttccgacgttccttcgacagctcgaatcgaaactcggatatcgctcaccgtgactcgttccaacccggcgctccgggagctttttggctcgttatcggcggagaaacgtctcccggcggttctcgtcgtcgatctatttggtacggatgcgttcgacgtggctgctgagttccacgtgtcgccatacattttctatgcatcaaatgccaacgtcctcacgtttctgcttcacttgccgaagctagacgaaacggtgtcgtgtgagtttagggaattaaccgaaccggttattattcccggttgtgtccccataaccggtaaggatttcgtcgatccgtgtcaagaccgaaaagatgaatcatacaaatggcttctacacaacgtcaagagattcaaagaagctgaagggattctagtgaattccttcgtcgatttagagccaaacactataaagattgtacaagaaccggctcctgataaaccaccggtttacctgattgggccgttggttaactcgggttcacacgatgctgacgtgaacgatgagtacaaatgtttaaattggctagacaaccaaccattcgggtcggttctatacgtatcctttggaagcggcggaacactcacgtttgagcagttcattgagctggctcttggcctagcggagagtggaaaacggtttctttgggtcatacgaagtccgagtgggatagctagttcatcgtatttcaatccacaaagccgaaatgatccattttcgtttttaccacaaggcttcttagaccgaaccaaagaaaaaggtctagtggttgggtcatgggctccacaggctcaaattctgactcatacatctataggtggatttttaactcattgtggatggaattcgagtctagaaagtattgtaaacggtgtaccgctcatagcatggccgttatacgcggagcaaaagatgaacgcattgctactcgtggatgttggtgcggctctaagagcacgactgggtgaagacggggtcgtaggaagggaagaagtggcgagagtggtaaaaggattgatagaaggagaagaagggaatgcggtaaggaaaaaaatgaaagagttgaaagaaggatctgttagagtcttaagggacgatggattctctaccaaatcgcttaatgaagtttcgttgaagtggaaagcccaccaacgaaagatcgaccaagaacaggaatcatttctatga

SEQ ID NO: 30 - protein, 481 aa, Arabidopsis thaliana
MADGNTPHVAIIPSPGIGHLIPLVELAKRLLDNHGFTVTFIIPGDSPPSKAQRSVLNSLPSSIASVFLPPADLSDVPSTARIETRISLTVTRSNPALRELFGSLSAEKRLPAVLVVDLFGTDAFDVAAEFHVSPYIFYASNANVLTFLLHLPKLDETVSCEFRELTEPVIIPGCVPITGKDFVDPCQDRKDESYKWLLHNVKRFKEAEGILVNSFVDLEPNTIKIVQEPAPDKPPVYLIGPLVNSGSHDADVNDEYKCLNWLDNQPFGSVLYVSFGSGGTLTFEQFIELALGLAESGKRFLWVIRSPSGIASSSYFNPQSRNDPFSFLPQGFLDRTKEKGLVVGSWAPQAQILTHTSIGGFLTHCGWNSSLESIVNGVPLIAWPLYAEQKMNALLLVDVGAALRARLGEDGVVGREEVARVVKGLIEGEEGNAVRKKMKELKEGSVRVLRDDGFSTKSLNEVSLKWKAHQRKIDQEQESFL

SEQ ID NO: 31 - DNA, 1413 bp, Arabidopsis thaliana
atggaccagcctcacgcgcttctagtggctagccctggcttgggtcacctcatccctatcctggagctcggcaaccgtctctcctccgtcctaaacatccacgtcaccattctcgcggtcacctccggctcctcttcaccgacagaaaccgaagccatacatgcagccgcggctagaacaatctgtcaaattacggaaattccctcggtggatgtagacaacctcgtggagccagatgctacaattttcactaagatggtggtgaagatgcgagccatgaagcccgcggtacgagatgccgtgaaattaatgaaacgaaaaccaacggtcatgattgttgactttttgggtacggaactgatgtccgtagccgatgacgtaggcatgacggctaaatacgtttacgttccaactcatgcgtggttcttggcagtcatggtgtacttgccggtgttagatacggtagtggaaggtgagtatgttgatattaaggagcctttgaagataccgggttgtaaaccggtcggaccgaaggagctgatggaaacgatgttagaccggtcgggccagcaatataaagagtgtgtacgagctggcttagaggtacctatgagcgatggtgttttggtaaatacttgggaggagttacaaggaaacactctcgctgcgcttagagaggacgaagaattgagccgggtcatgaaagtaccggtttatcctattgggccaattgttaggactaaccagcatgtagacaaacccaatagtatattcgagtggctagacgagcaacgggaaaggtcagtggtgtttgtgtgtttagggagcggtggaacgttgacgtttgagcaaacagtggaactcgctttgggtttagagttaagtggtcaaaggttcgtttgggttctacgtaggcccgcttcatatctcggggcgatctccagcgatgatgaacaggtaagtgccagtctacctgaaggtttcttggaccgcacgcgtggtgtggggattgtggttacgcaatgggcaccacaagttgagatcttgagccatagatcgatcggtgggttcttgtctcactgcggttggagttcggctttggaaagtttgactaaaggagttccgatcatcgcttggcctctttatgcggagcagtggatgaatgccacgttattgactgaggagatcggtgtggccgttcgtacatcggagttaccgtcggagagagtcatcggaagggaagaagtggcatctctggtgagaaagattatggcggaagaggatgaagaaggacagaaaattagggctaaagctgaggaggtgagggttagctccgaacgagcttggagtaaagacgggtcatcttataattctctattcgaatgggcaaaacgatgttatcttgtaccctag

SEQ ID NO: 32 - protein, 470 aa, Arabidopsis thaliana
MDQPHALLVASPGLGHLIPILELGNRLSSVLNIHVTILAVTSGSSSPTETEAIHAAAARTICQITEIPSVDVDNLVEPDATIFTKMVVKMRAMKPAVRDAVKLMKRKPTVMIVDFLGTELMSVADDVGMTAKYVYVPTHAWFLAVMVYLPVLDTVVEGEYVDIKEPLKIPGCKPVGPKELMETMLDRSGQQYKECVRAGLEVPMSDGVLVNTWEELQGNTLAALREDEELSRVMKVPVYPIGPIVRTNQHVDKPNSIFEWLDEQRERSVVFVCLGSGGTLTFEQTVELALGLELSGQRFVWVLRRPASYLGAISSDDEQVSASLPEGFLDRTRGVGIVVTQWAPQVEILSHRSIGGFLSHCGWSSALESLTKGVPIIAWPLYAEQWMNATLLTEEIGVAVRTSELPSERVIGREEVASLVRKIMAEEDEEGQKIRAKAEEVRVSSERAWSKDGSSYNSLFEWAKRCYLVP

SEQ ID NO: 33 - DNA, 1446 bp, Arabidopsis thaliana
atgcatatcacaaaaccacacgccgccatgttttccagtcccggaatgggccatgtcatcccggtgatcgagcttggaaagcgtctctccgctaacaacggcttccacgtcaccgtcttcgtcctcgaaaccgacgcagcctccgctcaatccaagttcctaaactcaaccggcgtcgacatcgtcaaacttccatcgccggacatttatggtttagtggaccccgacgaccatgtagtgaccaagatcggagtcattatgcgtgcagcagttccagccctccgatccaagatcgctgccatgcatcaaaagccaacggctctgatcgttgacttgtttggcacagatgcgttatgtctcgcaaaggaatttaacatgttgagttatgtgtttatccctaccaacgcacgttttctcggagtttcgatttattatccaaatttggacaaagatatcaaggaagagcacacagtgcaaagaaacccactcgctataccggggtgtgaaccggttaggttcgaagatactctggatgcatatctggttcccgacgaaccggtgtaccgggattttgttcgtcatggtctggcttacccaaaagccgatggaattttggtaaatacatgggaagagatggagcccaaatcattgaagtcccttctaaacccaaagctcttgggccgggttgctcgtgtaccggtctatccaatcggtcccttatgcagaccgatacaatcatccgaaaccgatcacccggttttggattggttaaacgaacaaccgaacgagtcggttctctatatctccttcgggagtggtggttgtctatcggcgaaacagttaactgaattggcgtggggactcgagcagagccagcaacggttcgtatgggtggttcgaccaccggtcgacggttcgtgttgtagcgagtatgtctcggctaacggtggtggaaccgaagacaacacgccagagtatctaccggaagggttcgtgagtcgtactagtgatagaggtttcgtggtcccctcatgggccccacaagctgaaatcctgtcccatcgggccgttggtgggtttttgacccattgcggttggagctcgacgttggaaagcgtcgttggcggcgttccgatgatcgcatggccactttttgccgagcagaatatgaatgcggcgttgctcagcgacgaactgggaatcgcagtcagattggatgatccaaaggaggatatttctaggtggaagattgaggcgttggtgaggaaggttatgactgagaaggaaggtgaagcgatgagaaggaaagtgaagaagttgagagactcggcggagatgtcactgagcattgacggtggtggtttggcgcacgagtcgctttgcagagtcaccaaggagtgtcaacggtttttggaacgtgtcgtggacttgtcacgtggtgcttag

SEQ ID NO: 34 - protein, 481 aa, Arabidopsis thaliana
MHITKPHAAMFSSPGMGHVIPVIELGKRLSANNGFHVTVFVLETDAASAQSKFLNSTGVDIVKLPSPDIYGLVDPDDHVVTKIGVIMRAAVPALRSKIAAMHQKPTALIVDLFGTDALCLAKEFNMLSYVFIPTNARFLGVSIYYPNLDKDIKEEHTVQRNPLAIPGCEPVRFEDTLDAYLVPDEPVYRDFVRHGLAYPKADGILVNTWEEMEPKSLKSLLNPKLLGRVARVPVYPIGPLCRPIQSSETDHPVLDWLNEQPNESVLYISFGSGGCLSAKQLTELAWGLEQSQQRFVWVVRPPVDGSCCSEYVSANGGGTEDNTPEYLPEGFVSRTSDRGFVVPSWAPQAEILSHRAVGGFLTHCGWSSTLESVVGGVPMIAWPLFAEQNMNAALLSDELGIAVRLDDPKEDISRWKIEALVRKVMTEKEGEAMRRKVKKLRDSAEMSLSIDGGGLAHESLCRVTKECQRFLERVVDLSRGA

SEQ ID NO: 35 - DNA, 1413 bp, Arabidopsis thaliana
atggaaaaaacaccccatatagctattgtaccaagtccaggaatgggacacttgatccctttggttgaatttgccaaaagattgaagaacaaccacaacatcgatgcaactttcatcattccaaatgatggacctctatccaaatctcaacgtgtttatctcgattcactcccaaccggattaaaccatatcattctccctccagttagtttcgatgatctaccacaagatgcaaagatggaaacccgaatcagcctcatggttacacgatctatcgatttccttcgagaagctttgaagtcattagttgcagaaacaaacatggtggcactgtttattgatctttttggtacagatgcatttgatgttgctattgaatttggtgtttcaccatatgtcttctttccatcaactgcaatggctttatctttgtttcttcatttaccaaaacttgatcaaatggtttcatgtgagtatagggacttgcctgaaccggttcagatcccgggttgcataccagttcccggtcgagacctacttgacccggttcaagatagaaagaacgaagcgtataagtgggtgcttcataacgcaaagaggtattcgatggctgagggtatagcggtaaatagcttcaaggagttagaaggtggagccttgaaagctttactagaggaagaaccgggcaaaccaaaggtttatccggttggaccgttgatacagaccggttcaagtactgatgttgatgggtccgagtgtttgaggtggttagacggtcagccatgtggttctgttttgtacgtatcttttggaagtggtggaaccttatcttctaatcagctcaatgagttagcctttggtttggaattaagtgagcaaaggttcatatgggtggttagaagcccgaatgatcaacccaacgcgacttactttaactcacatggtcatatggacccgttgggtttcttaccagaagggtttctagaaagaaccaaaggttttgggcttgtggttccttcttgggccccacaagcccaaatcttgagtcatagttcaaccggtgggtttttaacccactgtggttggaactcgattcttgagactgtagtccatggtgtgccggttatcgcctggccactttacgcagagcagaggatgaacgcggtatctttaaccgagggtataaaagtggcgttaaggcccaacgtggacgaaaatggcatcgtgggccgtgtggagattgcgagggtcgtgaagggtttgttagaaggggaagaaggaaaaccgattaggagtcgaattcgggatcttaaagatgcagctgctaatgttcttagtaaagatgggtgttccacaaaaactttagtgcagttggcttccaagttgaaaacgaagagtaaattaagcatttaa

SEQ ID NO: 36 - protein, 470 aa, Arabidopsis thaliana
MEKTPHIAIVPSPGMGHLIPLVEFAKRLKNNHNIDATFIIPNDGPLSKSQRVYLDSLPTGLNHIILPPVSFDDLPQDAKMETRISLMVTRSIDFLREALKSLVAETNMVALFIDLFGTDAFDVAIEFGVSPYVFFPSTAMALSLFLHLPKLDQMVSCEYRDLPEPVQIPGCIPVPGRDLLDPVQDRKNEAYKWVLHNAKRYSMAEGIAVNSFKELEGGALKALLEEEPGKPKVYPVGPLIQTGSSTDVDGSECLRWLDGQPCGSVLYVSFGSGGTLSSNQLNELAFGLELSEQRFIWVVRSPNDQPNATYFNSHGHMDPLGFLPEGFLERTKGFGLVVPSWAPQAQILSHSSTGGFLTHCGWNSILETVVHGVPVIAWPLYAEQRMNAVSLTEGIKVALRPNVDENGIVGRVEIARVVKGLLEGEEGKPIRSRIRDLKDAAANVLSKDGCSTKTLVQLASKLKTKSKLSI

SEQ ID NO: 37 - DNA, 1455 bp, Arabidopsis thaliana
atgaacagagaagtctctgagagaattcatattttgttcttccccttcatggctcaaggccacatgattccaattttggacatggccaagcttttctcgaggagaggagccaagtcaacccttctcacaaccccaatcaacgctaagatcttcgagaaacctattgaagcattcaaaaatcaaaaccctgatctcgaaatcggaatcaagatcttcaatttcccttgtgtagagcttggattgcctgaaggatgcgagaacgctgactttatcaactcataccaaaaatctgactcaggtgacttgttcttgaagtttcttttctctaccaagtatatgaaacaacagttggagagtttcattgaaacaaccaaaccaagtgctcttgttgccgatatgttcttcccttgggcgacagaatctgctgagaagctcggtgtaccaagacttgtgttccacggtacatctttcttttctttgtgttgttcgtataacatgaggattcataagccacacaagaaagtcgctacgagttctactccttttgtaatccctggtctcccaggagacatagttattacagaagaccaagccaatgttgccaaagaagaaacgccaatgggaaagtttatgaaagaggttagggaatcagagaccaatagctttggtgtattggttaatagcttctacgagctggaatcagcttatgctgatttttatcgtagttttgtggcgaaaagagcttggcatatcggtccgctttcgctatctaacagagagttaggagagaaagccagaagagggaaaaaggctaacattgatgagcaagaatgcctaaaatggctggactctaagacacctggttcagtagtttacttgtcctttgggagcggaactaatttcaccaacgaccagctgttagagatcgcttttggtcttgaaggttctggacaaagtttcatctgggtggttaggaaaaatgaaaaccaaggtgacaatgaagagtggttgcctgaagggtttaaagagaggacaacagggaaagggctaataatacctggatgggcgccgcaagtgctgatacttgaccataaagcaattggaggatttgtgactcattgcggatggaactcggctatagagggcattgccgcggggctgcctatggtaacatggccaatgggggcagaacagttctacaatgagaagctattgacaaaagtgttgagaataggagtgaacgttggagctaccgagttggtgaaaaaaggaaagttgattagtagagcacaagtggagaaggcagtaagggaagtgattggtggtgagaaggcagaggaaaggcggctatgggctaagaagctgggcgagatggctaaagccgctgtggaagaaggagggtcctcttataatgatgtgaacaagtttatggaagagctgaatggtagaaagtag

SEQ ID NO: 38 - protein, 484 aa, Arabidopsis thaliana
MNREVSERIHILFFPFMAQGHMIPILDMAKLFSRRGAKSTLLTTPINAKIFEKPIEAFKNQNPDLEIGIKIFNFPCVELGLPEGCENADFINSYQKSDSGDLFLKFLFSTKYMKQQLESFIETTKPSALVADMFFPWATESAEKLGVPRLVFHGTSFFSLCCSYNMRIHKPHKKVATSSTPFVIPGLPGDIVITEDQANVAKEETPMGKFMKEVRESETNSFGVLVNSFYELESAYADFYRSFVAKRAWHIGPLSLSNRELGEKARRGKKANIDEQECLKWLDSKTPGSVVYLSFGSGTNFTNDQLLEIAFGLEGSGQSFIWVVRKNENQGDNEEWLPEGFKERTTGKGLIIPGWAPQVLILDHKAIGGFVTHCGWNSAIEGIAAGLPMVTWPMGAEQFYNEKLLTKVLRIGVNVGATELVKKGKLISRAQVEKAVREVIGGEKAEERRLWAKKLGEMAKAAVEEGGSSYNDVNKFMEELNGRK

SEQ ID NO: 39 - DNA, 1362 bp, Arabidopsis thaliana
atggaggaaaagcctgcaaggagaagcgtagtgttggttccatttccagcacaaggacatatatctccaatgatgcaacttgccaaaacccttcacttaaagggtttctcgatcacagttgttcagactaagttcaattactttagcccttcagatgacttcactcatgattttcagttcgtcaccattccagaaagcttaccagagtctgatttcaagaatctcggaccaatacagtttctgtttaagctcaacaaagagtgtaaggtgagcttcaaggactgtttgggtcagttggtgctgcaacaaagtaatgagatctcatgtgtcatctacgatgagttcatgtactttgctgaagctgcagccaaagagtgtaagcttccaaacatcattttcagcacaacaagtgccacggctttcgcttgccgctctgtatttgacaaactatatgcaaacaatgtccaagctcccttgaaagaaactaaaggacaacaagaagagctagttccggagttttatcccttgagatataaagactttccagtttcacggtttgcatcattagagagcataatggaggtgtataggaatacagttgacaaacggacagcttcctcggtgataatcaacactgcgagctgtctagagagctcatctctgtcttttctgcaacaacaacagctacaaattccagtgtatcctataggccctcttcacatggtggcctcagctcctacaagtctgcttgaagagaacaagagctgcatcgaatggttgaacaaacaaaaggtaaactcggtgatatacataagcatgggaagcatagctttaatggaaatcaacgagataatggaagtcgcgtcaggattggctgctagcaaccaacacttcttatgggtgatccgaccagggtcaatacctggttccgagtggatagagtccatgcctgaagagtttagtaagatggttttggaccgaggttacattgtgaaatgggctccacagaaggaagtactttctcatcctgcagtaggagggttttggagccattgtggatggaactcgacactagaaagcatcggccaaggagttccaatgatctgcaggccattttcgggtgatcaaaaggtgaacgctagatacttggagtgtgtatggaaaattgggattcaagtggagggtgagctagacagaggagtggtcgagagagctgtgaagaggttaatggttgacgaagaaggagaggagatgaggaagagagctttcagtttaaaagagcaacttagagcctctgttaaaagtggaggctcttcacacaactcgctagaagagtttgtacacttcataaggactgcctag

SEQ ID NO: 40 - protein, 453 aa, Arabidopsis thaliana
MEEKPARRSVVLVPFPAQGHISPMMQLAKTLHLKGFSITVVQTKFNYFSPSDDFTHDFQFVTIPESLPESDFKNLGPIQFLFKLNKECKVSFKDCLGQLVLQQSNEISCVIYDEFMYFAEAAAKECKLPNIIFSTTSATAFACRSVFDKLYANNVQAPLKETKGQQEELVPEFYPLRYKDFPVSRFASLESIMEVYRNTVDKRTASSVIINTASCLESSSLSFLQQQQLQIPVYPIGPLHMVASAPTSLLEENKSCIEWLNKQKVNSVIYISMGSIALMEINEIMEVASGLAASNQHFLWVIRPGSIPGSEWIESMPEEFSKMVLDRGYIVKWAPQKEVLSHPAVGGFWSHCGWNSTLESIGQGVPMICRPFSGDQKVNARYLECVWKIGIQVEGELDRGVVERAVKRLMVDEEGEEMRKRAFSLKEQLRASVKSGGSSHNSLEEFVHFIRTA

SEQ ID NO: 41 - DNA, 1383 bp, Arabidopsis thaliana
atgaccaaaccctccgacccaaccagagactcccacgtggcagttctcgcttttcctttcggcactcatgcagctcctctcctcaccgtcacgcgccgcctcgcctccgcctctccttccaccgtcttctctttcttcaacaccgcacaatccaactcttcgttattttcctccggtgacgaagcagatcgtccggcgaacatcagagtatacgatattgccgacggtgttccggagggatacgtgtttagcgggagaccacaggaggcgatcgagctgtttcttcaagctgcgccggagaatttccggagagaaatcgcgaaggcggagacggaggttggtacggaagtgaaatgtttgatgactgatgcgttcttctggttcgcggctgatatggcgacggagataaatgcgtcgtggattgcgttttggaccgccggagcaaactcactctctgctcatctctacacagatctcatcagagaaaccatcggtgtcaaagaagtaggtgagcgtatggaggagacaataggggttatctcaggaatggagaagatcagagtcaaagatacaccagaaggagttgtgtttgggaatttagactctgttttctcaaagatgcttcatcaaatgggtcttgctttgcctcgtgccactgctgttttcatcaattcttttgaagatttggatcctacattgacgaataacctcagatcgagatttaaacgatatctgaacatcggtcctctcgggttattatcttctacattgcaacaactagtgcaagatcctcacggttgtttggcttggatggagaagagatcttctggttctgtggcgtacattagctttggtacggtcatgacaccgcctcctggagagcttgcggcgatagcagaagggttggaatcgagtaaagtgccgtttgtttggtcgcttaaggagaagagcttggttcagttaccaaaagggtttttggataggacaagagagcaagggatagtggttccatgggcaccgcaagtggaactgctgaaacacgaagcaacgggtgtgtttgtgacgcattgtggatggaactcggtgttggagagtgtatcgggtggtgtaccgatgatttgcaggccattttttggggatcagagattgaacggaagagcggtggaggttgtgtgggagattggaatgacgattatcaatggagtcttcacgaaagatgggtttgagaagtgtttggataaagttttagttcaagatgatggtaagaagatgaaatgtaatgctaagaaacttaaagaactagcttacgaagctgtctcttctaaaggaaggtcctctgagaatttcagaggattgttggatgcagttgtaaacattatctag

SEQ ID NO: 42 - protein, 460 aa, Arabidopsis thaliana
MTKPSDPTRDSHVAVLAFPFGTHAAPLLTVTRRLASASPSTVFSFFNTAQSNSSLFSSGDEADRPANIRVYDIADGVPEGYVFSGRPQEAIELFLQAAPENFRREIAKAETEVGTEVKCLMTDAFFWFAADMATEINASWIAFWTAGANSLSAHLYTDLIRETIGVKEVGERMEETIGVISGMEKIRVKDTPEGVVFGNLDSVFSKMLHQMGLALPRATAVFINSFEDLDPTLTNNLRSRFKRYLNIGPLGLLSSTLQQLVQDPHGCLAWMEKRSSGSVAYISFGTVMTPPPGELAAIAEGLESSKVPFVWSLKEKSLVQLPKGFLDRTREQGIVVPWAPQVELLKHEATGVFVTHCGWNSVLESVSGGVPMICRPFFGDQRLNGRAVEVVWEIGMTIINGVFTKDGFEKCLDKVLVQDDGKKMKCNAKKLKELAYEAVSSKGRSSENFRGLLDAVVNII

SEQ ID NO: 43 - DNA, 1422 bp, Arabidopsis thaliana
atgaaagtgaacgaggaaaacaacaagccgacaaagacccatgtcttaatcttcccatttccggcgcaaggtcacatgattcccctcctcgacttcacccaccgccttgctctccgcggcggcgccgccttaaaaataaccgtcctagtcactccaaaaaaccttccttttctctctccgcttctctccgccgtagttaacatcgaaccacttatcctcccttttccctcccacccttcaatcccctccggcgtcgaaaacgtccaagacttacctccttcaggcttccctttaatgatccacgcgcttggtaatctccacgcgccgcttatctcttggattacttctcacccttctcctccagtagccatcgtatctgatttcttccttggttggaccaaaaacctcggaatccctcgtttcgatttctctccctccgctgctatcacttgctgcatactcaatactctctggatcgaaatgcccaccaagatcaacgaagatgacgataacgagatcctccactttcccaagatcccgaattgtccaaaataccgttttgatcagatctcctctctttacagaagttacgttcacggagatccagcttgggagttcataagagactcctttagagataacgtggcgagttggggactcgtcgtgaactcgttcaccgccatggaaggtgtttatctcgaacatcttaagcgagagatgggccatgatcgtgtatgggctgtaggcccaattattccgttatctggggataaccgtggtggcccgacttctgtttctgttgatcacgtgatgtcgtggcttgacgcacgtgaggataaccacgtggtgtacgtgtgctttggaagtcaagtagttttgactaaagagcagactcttgcactcgcctctgggcttgagaaaagcggcgtccatttcatatgggccgtaaaggagcccgttgagaaagactcaacacgtggcaacatcctggacggtttcgacgatcgcgtggctgggagaggtctggtgatcagaggatgggctccacaagtagctgtgctacgtcaccgagccgttggcgcgtttttaacgcactgtggttggaactctgtggtggaggcggttgtcgccggcgttttgatgctgacgtggccgatgagagctgaccagtacactgacgcgtctctggtggttgatgagttgaaagtaggtgtgcgtgcttgcgaaggacctgacacggtgcctgacccggacgagttagctcgagttttcgctgattccgtgaccggaaatcaaacggagaggatcaaagccgtggagctgaggaaagcagcgttggatgcgattcaagaacgtgggagctcagtgaatgatttagatggatttatccaacatgtcgttagtttaggactaaaccgctag

SEQ ID NO: 44 - protein, 473 aa, Arabidopsis thaliana
MKVNEENNKPTKTHVLIFPFPAQGHMIPLLDFTHRLALRGGAALKITVLVTPKNLPFLSPLLSAVVNIEPLILPFPSHPSIPSGVENVQDLPPSGFPLMIHALGNLHAPLISWITSHPSPPVAIVSDFFLGWTKNLGIPRFDFSPSAAITCCILNTLWIEMPTKINEDDDNEILHFPKIPNCPKYRFDQISSLYRSYVHGDPAWEFIRDSFRDNVASWGLVVNSFTAMEGVYLEHLKREMGHDRVWAVGPIIPLSGDNRGGPTSVSVDHVMSWLDAREDNHVVYVCFGSQVVLTKEQTLALASGLEKSGVHFIWAVKEPVEKDSTRGNILDGFDDRVAGRGLVIRGWAPQVAVLRHRAVGAFLTHCGWNSVVEAVVAGVLMLTWPMRADQYTDASLVVDELKVGVRACEGPDTVPDPDELARVFADSVTGNQTERIKAVELRKAALDAIQERGSSVNDLDGFIQHVVSLGLNR

SEQ ID NO: 45 - DNA, 1404 bp, Arabidopsis thaliana
atggagttagaaaaagttcacgtggttttgttcccatacttgtccaaagggcacatgattcctatgctccaattagctcgtctcctcttatcccactccttcgccggagacatctccgtcaccgtcttcaccactcctttgaaccgtcctttcatcgttgactcactctccggcaccaaagcgaccatcgtcgacgtacctttccctgataacgtcccggagatcccacccggcgtcgagtgcactgacaaactccctgctttgtcgtcctccctcttcgttcctttcacaagagccaccaagtcaatgcaggcagactttgagcgagagctcatgtcactgccacgtgtcagtttcatggtctcagacggtttcttgtggtggacgcaagagtcagctcgaaagctagggtttcctcggcttgttttctttggtatgaattgcgcttccaccgttatatgtgacagtgtttttcaaaaccagcttctatctaatgttaagtccgagacggagccagtttctgtaccggagtttccgtggattaaggttaggaaatgtgatttcgttaaagatatgtttgatccaaaaaccaccacagatcctggattcaagcttatcctagatcaagtcacgtctatgaatcaaagccaaggtatcatattcaatacatttgacgaccttgaacccgtgtttattgatttctacaagcgtaaacgcaaactcaagctttgggcagttggaccgctttgttacgtaaataacttcttggatgatgaagtagaagagaaggtcaaacctagttggatgaaatggctagatgaaaagcgagacaagggatgcaatgttctgtatgtggctttcgggtcacaagccgagatctcgagagaacaactagaggagattgcgttagggttggaagaatcgaaggtgaacttcttgtgggtggtcaaaggaaatgaaataggaaaagggtttgaagagagagtgggagaaagaggaatgatggtgagagatgaatgggttgatcagaggaagatattagagcacgagagtgttagagggttcttgagccattgtgggtggaattctctgacggagagcatttgctcggaggttccaatcttggcgtttcctttagcagcggagcaacctctgaatgcgattttggtggtggaagagctgagagtggcggagagagtggtggcggcgagtgaaggggttgtgagaagagaagagattgcagagaaagtgaaggagttgatggagggagagaaagggaaagagctgaggaggaatgtcgaggcatatggtaagatggcgaagaaggctttggaggaaggtattggttcgtctaggaagaatttagacaaccttatcaacgagttttgtaacaatggaacatga

SEQ ID NO: 46 - protein, 467 aa, Arabidopsis thaliana
MELEKVHVVLFPYLSKGHMIPMLQLARLLLSHSFAGDISVTVFTTPLNRPFIVDSLSGTKATIVDVPFPDNVPEIPPGVECTDKLPALSSSLFVPFTRATKSMQADFERELMSLPRVSFMVSDGFLWWTQESARKLGFPRLVFFGMNCASTVICDSVFQNQLLSNVKSETEPVSVPEFPWIKVRKCDFVKDMFDPKTTTDPGFKLILDQVTSMNQSQGIIFNTFDDLEPVFIDFYKRKRKLKLWAVGPLCYVNNFLDDEVEEKVKPSWMKWLDEKRDKGCNVLYVAFGSQAEISREQLEEIALGLEESKVNFLWVVKGNEIGKGFEERVGERGMMVRDEWVDQRKILEHESVRGFLSHCGWNSLTESICSEVPILAFPLAAEQPLNAILVVEELRVAERVVAASEGVVRREEIAEKVKELMEGEKGKELRRNVEAYGKMAKKALEEGIGSSRKNLDNLINEFCNNGT

SEQ ID NO: 47 - DNA, 1413 bp, Rauvolfia serpentina
atggagcatacacctcacattgctatggtgcccactccgggaatgggtcatctgatccccctcgttgagttcgctaaacgactcgtcctccgtcacaactttggcgtcacttttattatcccaaccgatggacctctccctaaagcacagaagagttttcttgatgctcttcccgccggcgtaaactatgttcttcttcccccggtaagcttcgacgacttacccgctgatgttaggatagagacccgtatttgtctcaccatcactcgctctctcccgtttgttcgggatgccgttaagactctactcgccaccaccaagttagctgctctagtggtggatcttttcggcaccgatgcatttgatgttgcaattgagttcaaggtctccccttatatcttctatcctacgacggccatgtgcctgtctcttttctttcacttgcctaagcttgatcaaatggtgtcctgcgaatatagagacgtcccagaaccattgcagattccaggatgcatacccattcacgggaaggattttcttgacccagctcaggatcgcaaaaatgatgcctacaaatgcctccttcaccaggccaagagataccggttagctgagggtatcatggtcaacaccttcaacgacttggagccaggacccttaaaagctttgcaggaggaagaccagggtaagccacccgtttatccgatcggaccactcatcagagcggattcaagcagcaaggtcgacgactgtgaatgtttgaaatggctagatgaccagccacgtgggtcggttctgtttatttctttcggaagcggtggggcagtctaccataatcagttcattgagctagctttgggattagagatgagcgagcaaagattcttgtgggttgtccgaagcccaaatgataaaattgcgaatgcaacgtatttcagcattcaaaatcagaatgatgctcttgcatatctgccagaaggattcttggagagaaccaaggggcgttgtcttttggtcccgtcttgggcgccgcagactgaaattcttagccatggttccacgggtggatttctaacccactgcgggtggaactctattcttgagagtgtagttaatggggtgccgctaattgcttggcctctttatgcagagcaaaagatgaacgccgtaatgttgacggagggtcttaaagtggccctgaggccaaaagccggtgaaaatggcttgataggccgagtcgagatcgccaatgccgttaagggcttaatggagggagaggaaggaaagaagttccgcagcacaatgaaagacctaaaagatgcggcatcgagggcgctaagtgatgacggttcttcgacaaaagcactcgctgaattggcttgcaagtgggagaacaaaatgtccagtacctag

SEQ ID NO: 48 - protein, 470 aa, Rauvolfia serpentina
MEHTPHIAMVPTPGMGHLIPLVEFAKRLVLRHNFGVTFIIPTDGPLPKAQKSFLDALPAGVNYVLLPPVSFDDLPADVRIETRICLTITRSLPFVRDAVKTLLATTKLAALVVDLFGTDAFDVAIEFKVSPYIFYPTTAMCLSLFFHLPKLDQMVSCEYRDVPEPLQIPGCIPIHGKDFLDPAQDRKNDAYKCLLHQAKRYRLAEGIMVNTFNDLEPGPLKALQEEDQGKPPVYPIGPLIRADSSSKVDDCECLKWLDDQPRGSVLFISFGSGGAVYHNQFIELALGLEMSEQRFLWVVRSPNDKIANATYFSIQNQNDALAYLPEGFLERTKGRCLLVPSWAPQTEILSHGSTGGFLTHCGWNSILESVVNGVPLIAWPLYAEQKMNAVMLTEGLKVALRPKAGENGLIGRVEIANAVKGLMEGEEGKKFRSTMKDLKDAASRALSDDGSSTKALAELACKWENKMSST

SEQ ID NO: 49 - DNA, 1380 bp, Nicotiana tabacum
atgactactcaaaaagctcattgcttgatcttaccatatccagctcagggtcatatcaaccctatgctccaattctccaaacgtttgcaatccaaaggtgtcaaaatcactatagcagccaccaaatcattcttgaaaaccatgcaagaattgtcaacttctgtgtcagtcgaggctatctccgatggctatgatgatggcggacgcgagcaagctggaacctttgtggcctatattacaagattcaaagaagttggctcggatactttgtctcagcttattggaaagttaacaaattgtggttgtcctgtgagttgcatagtttacgatccatttcttccttgggctgttgaagtgggaaataattttggagtagctactgctgcttttttcactcaatcttgtgcagtggataacatttattaccatgtacataaaggggttctaaaacttcctccaactgacgttgataaagaaatctcaattcctggattattaacaattgaggcatcagatgtacctagttttgtttctaatcctgaatcttcaagaatacttgaaatgttggtgaatcagttctcgaatcttgagaacacagattgggtcctaatcaacagtttctatgaattggagaaagaggtaattgattggatggccaagatctatccaatcaagacaattggaccaactataccatcaatgtacctagacaagaggctaccagatgacaaagaatatggccttagtgtcttcaagccaatgacaaatgcatgcctaaactggttaaaccatcaaccagttagctcagtagtatatgtatcatttggaagtttagccaaattagaagcagagcaaatggaagaattagcatggggtttgagtaatagcaacaagaacttcttgtgggtagttagatccactgaagaatccaaacttcccaacaactttttagaggaattagcaagtgaaaaaggattagtcgtgtcatggtgtccacaattacaagtcttggaacataaatcaatagggtgttttctcacgcactgtggctggaattcaactttggaagcaattagtttgggagtaccaatgattgcaatgccacattggtcagaccagccaacaaatgcgaagcttgtggaagatgtttgggagatgggaattagaccaaaacaagatgaaaaaggattagttagaagagaagttattgaagaatgtattaagatagtgatggaggaaaagaaaggaaaaaagattagggaaaatgcaaagaaatggaaggaattggctaggaaagctgtggatgaaggaggaagttcagatagaaatattgaagaatttgtttccaagttggtgactattgcctcagtggaaagctaa

SEQ ID NO: 50 - protein, 459 aa, Nicotiana tabacum
MTTQKAHCLILPYPAQGHINPMLQFSKRLQSKGVKITIAATKSFLKTMQELSTSVSVEAISDGYDDGGREQAGTFVAYITRFKEVGSDTLSQLIGKLTNCGCPVSCIVYDPFLPWAVEVGNNFGVATAAFFTQSCAVDNIYYHVHKGVLKLPPTDVDKEISIPGLLTIEASDVPSFVSNPESSRILEMLVNQFSNLENTDWVLINSFYELEKEVIDWMAKIYPIKTIGPTIPSMYLDKRLPDDKEYGLSVFKPMTNACLNWLNHQPVSSVVYVSFGSLAKLEAEQMEELAWGLSNSNKNFLWVVRSTEESKLPNNFLEELASEKGLVVSWCPQLQVLEHKSIGCFLTHCGWNSTLEAISLGVPMIAMPHWSDQPTNAKLVEDVWEMGIRPKQDEKGLVRREVIEECIKIVMEEKKGKKIRENAKKWKELARKAVDEGGSSDRNIEEFVSKLVTIASVES

SEQ ID NO: 51 - DNA, 1371 bp, Solanum lycopersicum
atgactactcacaaagctcattgcttaattttgccatttccaggccaaggtcatatcaacccaatgcttcaattctccaaacgtttacaatccaaacgcgttaaaatcactatagcactcacaaaatcctgtttgaaaacaatgcaagaattgtcaacttcagtatcaatcgaggcgatttctgatggctacgatgatggtggtttccatcaagcagaaaatttcgtagcctacataacacgattcaaagaagttggttcggatactctgtctcagcttattaaaaaattggaaaatagtgattgtcctgtaaattgcatagtatatgatccattcattccttgggctgttgaagttgcaaaacaatttggattaattagtgctgcatttttcacacaaaattgtgtagtggataatctttattaccatgtacataaaggggtgataaaacttccacctactcaaaatgacgaagaaatattaattcctggatttccaaattcgatcgatgcatcagatgtaccttcttttgttattagtcctgaagcagaaaggatagttgaaatgttagcaaatcaattctcaaatcttgacaaagttgattatgttctaatcaatagcttctatgagttggagaaagaggtaaatgaatggatgtcaaagatatatccaataaagacaattggaccaacaataccatcaatgtacttagacaagagactacatgatgataaagagtatggtcttagtgtcttcaagccaatgacaaatgaatgtctaaattggttaaaccatcaaccaattagctcagtggtgtatgtatcatttggaagtataaccaaattaggagatgagcaaatggaagaattggcatggggtttgaagaatagcaacaagagcttcttgtgggttgttaggtctactgaagagcccaaacttcccaacaactttattgaggaattaacaagtgaaaaaggcttagtggtgtcatggtgtccacaattacaagtgttggaacatgaatcgacaggttgttttctgacgcactgtggatggaattcaactctggaagcgattagtttgggagtgccaatggtggcaatgccacaatggtctgatcaaccaacaaatgcaaagcttgtgaaagatgtttgggaaataggtgttagagccaaacaagatgaaaaaggggtagttagaagagaagttatagaagaatgtataaagctagtgatggaagaagataaaggaaaactaattagagaaaatgcaaagaaatggaaggaaatagctagaaatgttgtgaatgaaggaggaagttcagataaaaacattgaagaatttgtttccaagttggttactatttcctaa

SEQ ID NO: 52 - protein, 456 aa, Solanum lycopersicum
MTTHKAHCLILPFPGQGHINPMLQFSKRLQSKRVKITIALTKSCLKTMQELSTSVSIEAISDGYDDGGFHQAENFVAYITRFKEVGSDTLSQLIKKLENSDCPVNCIVYDPFIPWAVEVAKQFGLISAAFFTQNCVVDNLYYHVHKGVIKLPPTQNDEEILIPGFPNSIDASDVPSFVISPEAERIVEMLANQFSNLDKVDYVLINSFYELEKEVNEWMSKIYPIKTIGPTIPSMYLDKRLHDDKEYGLSVFKPMTNECLNWLNHQPISSVVYVSFGSITKLGDEQMEELAWGLKNSNKSFLWVVRSTEEPKLPNNFIEELTSEKGLVVSWCPQLQVLEHESTGCFLTHCGWNSTLEAISLGVPMVAMPQWSDQPTNAKLVKDVWEIGVRAKQDEKGVVRREVIEECIKLVMEEDKGKLIRENAKKWKEIARNVVNEGGSSDKNIEEFVSKLVTIS

SEQ ID NO: 53 - DNA, 6536 bp, Escherichia coli
ctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacagctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaagtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggggaattgtgagcggataacaatttccctctagaaataattttgtttaaactttaagaaggagatatacatatgcaccatcatcatcatcattctggatccatggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcgggcccattttgaaccttgaaaacaaaaaagacgatgctaaaaccgacgagattatgaggtggttaaatgagcaaccggaaagctcggttgtgtttttatgtttcggaagcatgggtagctttaacgagaaacaagtgaaggagattgcggttgcgattgaaagaagtggacatagatttttatggtcgcttcgtcgtccgacaccgaaagaaaagatagagtttccgaaagaatatgaaaacttggaagaagttcttccagagggattccttaaacgtacatcaagcatcgggaaggtgatcgggtgggccccacaaatggcggtgttgtctcacccgtcagttggtgggtttgtgtcgcattgtggttggaactcgacattggagagtatgtggtgtggggttccgatggcagcttggccattatatgctgaacaaacgttgaatgcttttctacttgtggtggaactgggattggcggcggagattaggatggattatcggacggatacgaaagcggggtatgacggtgggatggaggtgacggtggaggagattgaagatggaattaggaagttgatgagtgatggtgagattagaaataaggtgaaagatgtgaaagagaagagtagagctgcggttgttgaaggtggatcttcttacgcatccattggaaaattcatcgagcatgtatcgaatgttacgatttaaggtcgacaagcttggcggccgcgccacgcgatcgctgacgtcggtaccctcgagtctggtaaagaaaccgctgctgcgaaatttgaacgccagcacatggactcgtctactagcgcagcttaattaacctagg